(19) United States
(12) Patent Application Publication
(10) Pub. No.: US 2024/0225828 A1
(43) Pub. Date: Jul. 11, 2024

(54) SYSTEMS AND METHODS FOR RETRIEVING SIDE-DELIVERABLE TRANSCATHETER PROSTHETIC VALVES

(71) Applicant: VDyne, Inc., Maple Grove, MN (US)

(72) Inventors: Robert VIDLUND, Forest Lake, MN (US); Mark CHRISTIANSON, Plymouth, MN (US); Craig EKVALL, East Bethel, MN (US); Cameron VIDLUND, Forest Lake, MN (US); David HOLTAN, Eden Prairie, MN (US); Neelakantan SAIKRISHNAN, Plymouth, MN (US)

(73) Assignee: VDyne, Inc., Maple Grove, MN (US)

(21) Appl. No.: 18/612,015

(22) Filed: Mar. 21, 2024

Related U.S. Application Data
(63) Continuation-in-part of application No. PCT/US23/63044, filed on Feb. 22, 2023.
(60) Provisional application No. 63/313,490, filed on Feb. 24, 2022.

(51) Int. Cl. A61F 2/24; A61F 2/95

(52) U.S. Cl. CPC A61F 2/2427; A61F 2/9517; A61F 2002/9528; A61F 2210/0014

(57) ABSTRACT

A delivery/retrieval system includes a control device and a retrieval element. The control device is removably coupleable to a side-deliverable prosthetic valve and is operable to (i) exert a distally directed force to advance the compressed valve through a delivery sheath and into a chamber of the heart and (ii) exert a proximally directed force to pull the expanded valve from the chamber of the heart into a retrieval sheath. A proximal end portion of the retrieval element is coupleable to a proximal end portion of the control device. A distal end portion of the retrieval element forms an engagement member operable to engage and exert a proximally directed force on a proximal subannular anchoring element of the valve to pull the valve into the retrieval sheath. A guide member is operable to guide at least one edge of the valve as it is pulled into the retrieval sheath.

### CROSS-REFERENCE TO RELATED APPLICATIONS

This application is a continuation-in-part of International Patent Application No. PCT/US2023/063044, filed Feb. 22, 2023, entitled “Systems and Methods for Retrieving Side-Deliverable Transcatheter Prosthetic Valves,” which claims priority to and the benefit of U.S. Provisional Patent Application No. 63/313,490, filed Feb. 24, 2022, entitled “Systems and Methods for Retrieving Side-Deliverable Transcatheter Prosthetic Valves,” the disclosure of each of which is incorporated herein by reference in its entirety.

### BACKGROUND

Embodiments described herein relate generally to transcatheter prosthetic valves and more particularly, to systems and methods for retrieving side-deliverable transcatheter prosthetic valves.

Prosthetic heart valves can pose challenges for delivery, deployment, and/or retrieval within a heart, particularly for delivery by catheters through the patient's vasculature rather than through a surgical approach. Delivery of traditional transcatheter prosthetic valves generally includes compressing the valve in a radial direction and loading the valve into a delivery catheter such that a central annular axis of the valve is parallel to a lengthwise or longitudinal axis of the delivery catheter. The valves are deployed from an end of the delivery catheter and expanded outwardly in a radial direction from the central annular axis. The expanded size (e.g., diameter) of traditional valves, however, can be limited by the internal diameter of the delivery catheter, which in turn, is limited by the vasculature of the patient. The competing interest of minimizing delivery catheter size presents challenges to increasing the expanded diameter of traditional valves (e.g., trying to compress too much material and structure into too little space). Moreover, the orientation of the traditional valves during deployment can create additional challenges when trying to align the valves with the native valve annulus.

Some transcatheter prosthetic valves can be configured for side and/or orthogonal delivery, which can allow for an increase in an expanded diameter relative to traditionally delivered valves. With side delivery, for example, the valve can be placed in a compressed or delivery configuration and loaded into a delivery catheter such that a central annular axis of the valve is substantially perpendicular and/or orthogonal to the lengthwise or longitudinal axis of the delivery catheter. More particularly, the valve can be compressed axially (e.g., along the central annular axis) and laterally (e.g., perpendicular to each of the central annular axis and a longitudinal axis of the valve), and uncompressed or elongated longitudinally (e.g., in a direction parallel to the lengthwise or longitudinal axis of the delivery catheter). The compressed valve (e.g., the valve in a delivery configuration) can be loaded into a lumen of the delivery catheter in a side-ways or orthogonal orientation, advanced through the lumen, and deployed from the end of the delivery catheter. Furthermore, in some instances, the sideways or orthogonal orientation of the deployed side-delivered valve relative to and/or within the delivery catheter, in general, results in the valve being deployed in a desired orientation relative to the native valve annulus.

In some instances, however, challenges associated with placing the valve in the native annulus, deterioration of the patient's condition, identification of device defects, and/or the like may make it desirable to retrieve or at least partially retrieve the valve prior to completely seating the valve in the native annulus. In some such instances, returning the valve to the compressed or delivery configuration and retracting the valve into the delivery catheter or other conduit can be difficult. For example, in some implementations, loading a valve into a delivery catheter can be assisted by various devices that compress the valve to a size suitable for disposal in the lumen of the delivery catheter. These devices, however, are not present inside the heart to assist in compressing the valve for retrieval, which can result in a relatively high amount of force being needed to compress and at least partially retrieve the valve. Moreover, in some instances, one or more portions, edges, surfaces, etc. of the valve can get caught on a distal edge of the delivery catheter (or separate retrieval catheter), which in turn, can prevent or hinder retraction of the valve into the delivery catheter.

Accordingly, a need exists for systems and methods for retrieving or at least partially retrieving side-deliverable transcatheter prosthetic valves from a chamber of a heart.

### SUMMARY

The embodiments described herein are directed to side-deliverable transcatheter prosthetic heart valves and devices, systems, and/or methods for retrieving the side-deliverable prosthetic valves. In some embodiments, a delivery/retrieval system for a side-deliverable prosthetic valve includes at least a control device and a retrieval element. The control device is configured to attach to the prosthetic valve. The control device is operable to (i) exert a distally directed force to advance the prosthetic valve in a compressed configuration through a lumen of a delivery sheath and into a chamber of the heart and (ii) exert a proximally directed force to pull the prosthetic valve disposed in the chamber of the heart in an expanded configuration into a distal end of a retrieval sheath. The retrieval element is extendable through the lumen of the retrieval sheath. A proximal end portion of the retrieval element is coupleable to a proximal end portion of the control device. A distal end portion of the retrieval element forms an engagement member and a guide member. The engagement member is configured to be engaged with and exert a proximally directed force on a proximal subannular anchoring element of the prosthetic valve to pull the prosthetic valve from the chamber of the heart into the distal end of the retrieval sheath. The guide member is configured to guide at least one edge of the prosthetic valve as the prosthetic valve is pulled into the distal end of the retrieval sheath.

### BRIEF DESCRIPTION OF THE DRAWINGS

FIGS. 1-5 are schematic illustrations of a side-deliverable transcatheter prosthetic valve selectively coupled to a delivery and/or retrieval system (or portions thereof), according to an embodiment.

FIGS. 6-9 are schematic illustrations of the prosthetic valve of FIG. 1 shown during an optional retrieval process.

FIG. 10 is an elevated side perspective view of a prosthetic valve according to an embodiment.

FIG. 11 is a bottom perspective view of the prosthetic valve shown in FIG. 10.

FIG. 12 is an elevated side perspective view of a supra-annular region of an outer support frame of the prosthetic valve shown in FIG. 10.

FIG. 13 is a distal perspective view a transannular region of the outer support frame of the prosthetic valve shown in FIG. 10.

FIG. 14 is a distal perspective view a subannular region of the outer support frame of the prosthetic valve shown in FIG. 10.

FIG. 15 is a top perspective view an inner frame of a flow control component included in the prosthetic valve shown in FIG. 10.

FIG. 16 is a side perspective view of a leaflet band of the inner flow control component having leaflet pockets sewn into a structural band and shown in a cylindrical configuration suitable for coupling to the inner frame of FIG. 15.

FIG. 17 is a bottom view of the leaflet band of FIG. 16 in the cylindrical configuration and showing partial coaptation of the leaflets to form a partially closed fluid-seal.

FIG. 18 is a side perspective view of the prosthetic valve of FIG. 10 removably coupled to a distal end portion of a control device included in a delivery system.

FIGS. 19 and 20 are bottom perspective views of the prosthetic valve of FIG. 10 and illustrating a process of transitioning a proximal anchoring element of the prosthetic valve between a first configuration and a second configuration, respectively, in response to actuation of an actuator such as one or more tethers.

FIGS. 21-24 illustrate a process of deploying a side-deliverable transcatheter prosthetic valve in an annulus of a native valve of the human heart according to an embodiment.

FIGS. 25-28 are various views of a delivery portion of the delivery/retrieval system, according to an embodiment, shown being decoupled from a control device/catheter to allow a retrieval portion of the delivery/retrieval system to be advanced over the control device/catheter and manipulated to retrieve a prosthetic valve.

FIGS. 29 and 30 are various views of an exchange catheter configured to engage a control catheter of the control device shown in FIGS. 25-28 and to allow at least a portion of the delivery/retrieval system to be advanced over the control device.

FIG. 31 is a side perspective view of the retrieval sheath included in the delivery/retrieval system of FIGS. 25-30 and shown with a retrieval handle coupled to a proximal end portion thereof.

FIG. 32 is an enlarged side view of a part of the retrieval handle of FIG. 31 coupled to the retrieval sheath.

FIG. 33 is a side view of a distal end portion of the retrieval sheath of FIG. 31.

FIG. 34 is a side view of a distal end portion of the delivery/retrieval system showing the retrieval element, a guide member, and an engagement member disposed, for example, in the retrieval sheath of FIG. 33 with at least the guide member in a compressed or at least partially compressed state for advancement into a chamber of the heart.

FIG. 35 is a top view of the distal end portion of the delivery/retrieval system showing the retrieval element, the engagement member, the guide member, and an optional actuator.

FIGS. 36 and 37 are side views of the distal end portion of the delivery/retrieval system showing the retrieval element, the engagement member, the guide member, and the optional actuator, with the guide member shown in an expanded/extended/unactuated state and a contracted/tensioned/actuated state, respectively.

FIGS. 38 and 39 are side perspective views of a distal portion of the delivery/retrieval system of FIGS. 25-37 selectively engaging a prosthetic valve.

FIGS. 40 and 41 are side view fluoroscopic black and white photographs showing the delivery/retrieval system of FIGS. 25-39 engaging the prosthetic valve.

FIG. 42 is a side view of a retractor included in the delivery/retrieval system of FIGS. 25-41.

FIG. 43 is a side view of the retractor of FIG. 42 temporarily coupled to the retrieval handle, a proximal end portion of the control device, and a proximal end portion of the retrieval element.

FIG. 44 is a flowchart illustrating a method of retrieving a side-deliverable transcatheter prosthetic valve from an atrium of a heart according to an embodiment.

### DETAILED DESCRIPTION

Disclosed embodiments are directed to side-deliverable transcatheter prosthetic heart valves and/or components thereof, and methods of deploying and/or retrieving the prosthetic valves and/or components thereof. In some embodiments, a delivery/retrieval system for a side-deliverable prosthetic valve includes at least a control device and a retrieval element. The control device can attach to the prosthetic valve. The control device is operable to (i) exert a distally directed force to advance the prosthetic valve in a compressed configuration through a lumen of a delivery sheath and into a chamber of the heart and (ii) exert a proximally directed force to pull the prosthetic valve disposed in the chamber of the heart in an expanded configuration into a distal end of a retrieval sheath. The retrieval element is extendable through the lumen of the retrieval sheath. A proximal end portion of the retrieval element is coupleable to a proximal end portion of the control device. A distal end portion of the retrieval element forms an engagement member and a guide member. The engagement member can be engaged with and exert a proximally directed force on a proximal subannular anchoring element of the prosthetic valve to pull the prosthetic valve from the chamber of the heart into the distal end of the retrieval sheath. The guide member can guide at least one edge of the prosthetic valve as the prosthetic valve is pulled into the distal end of the retrieval sheath.

In some embodiments, a delivery/retrieval system for a side-deliverable prosthetic valve includes at least a retrieval sheath defining a lumen, a control device extendable through the lumen of the retrieval sheath, and a retrieval element extendable through the lumen of the retrieval sheath and outside of the control device. The control device is removably coupleable to a supra-annular surface of the prosthetic valve. A distal end portion of the retrieval element includes an engagement member that can engage a proximal subannular anchoring element of the prosthetic valve when the prosthetic valve is at least partially disposed in the chamber of the heart. The arrangement of the control device being removably coupled to the supra-annular surface of the prosthetic valve and the engagement member being engaged with the proximal subannular anchoring element allows the control device and the retrieval element to pull the prosthetic valve into a distal end of the retrieval sheath in response to a proximally directed force.

In some embodiments, an apparatus for retrieving a side-deliverable prosthetic valve from a chamber of a heart of a patient includes at least a retrieval catheter, a guide member, and an engagement member. Each of the guide member and the engagement member being coupled to and extending distally from a distal end of the retrieval catheter. The guide member is formed from a braided tube of a shape-memory alloy. The guide member has a compressed state for delivery through a retrieval sheath into the chamber of the heart and an expanded state when in the chamber of the heart and distal to the retrieval sheath. The engagement member is partially embedded in the guide member such that a portion of the engagement member extends outwardly from the guide member allowing the portion of the engagement member to engage a subannular region of the prosthetic valve.

In some instances, it may be desirable to retrieve any of the prosthetic valves after at least partial deployment, full deployment, and/or implantation. For example, in some implementations, a method of using a delivery/retrieval system for retrieving a side-deliverable prosthetic valve at least partially disposed in a chamber of a heart of a patient includes decoupling a delivery portion of the delivery/retrieval system from a proximal end portion of a control device disposed outside the patient. A distal end portion of the control device is disposed in the chamber of the heart and is removably coupled to a supra-annular surface of the prosthetic valve. A retrieval sheath is advanced over the control device to place a distal end portion of a retrieval element distal to the retrieval sheath in the chamber of the heart. The distal end portion of the retrieval element includes an engagement member. A first portion of a retractor is coupled to a proximal end portion of the retrieval sheath and a second portion of the retractor is coupled to a proximal end portion of each of the control device and the retrieval element. A proximal subannular anchoring element of the prosthetic valve is engaged with the engagement member of the retrieval element and the retractor is actuated to move each of the control device and the retrieval element in a proximal direction relative to the retrieval sheath. The movement of each of the control device and the retrieval element exerting a proximally directed force on the prosthetic valve operable to pull the prosthetic valve into a distal end of the retrieval sheath.

Any of the prosthetic valves described herein can be relatively low-profile, transcatheter prosthetic heart valves. The prosthetic heart valves herein can have a valve frame and a flow control component mounted within a central lumen, aperture, and/or channel of the valve frame that extends along a central axis of the valve or valve frame that is co-axial or at least substantially parallel with a blood flow direction through the valves. The valve frame can provide structural support for the prosthetic valve and/or at least the flow control component mounted thereto. The valve frame can also provide one or more components or elements for anchoring or otherwise securing the prosthetic valves in an annulus of a native valve. The flow control component (e.g., a 2-leaflet or 3-leaflet sleeve, valve, and/or the like) can be configured to permit blood flow in a first direction through an inflow end of the valve and out an outflow end of the valve, and block blood flow in a second direction, opposite the first direction.

Any of the delivery and/or retrieval systems and/or methods described herein can be used and/or implemented for traditionally deliverable valves or orthogonal/side-deliverable valves unless clearly stated otherwise. For example, the valves described herein can be configured to transition (e.g., via balloon inflation or via one or more self-expanding structures) between a compressed or delivery configuration for introduction into the body via a delivery catheter, and an expanded or deployment/deployed configuration for implanting at a desired location in the body. The delivery catheter can be, for example, a 24-36 French (Fr) delivery catheter that is advanced through the vasculature of a patient and into a chamber of a heart. In general, traditionally-delivered/deliverable valves are configured to be compressed in, for example, a radial direction relative to the central axis or blood flow direction through the valve, and inserted into and/or advanced through the delivery catheter such that the central axis of the compressed valve is parallel to a longitudinal or lengthwise axis of the delivery catheter used to deliver the valve. The valves are deployed from the end of the delivery catheter and expanded outwardly in a radial direction from the central cylinder axis. The delivery orientation of the valve generally means that the valve is completely released from the delivery catheter while in the atrium of the heart and reoriented relative to the annulus, which in some instances, can limit a size of the valve. Accordingly, in some implementations, traditional delivery can be used for relatively small diameter valves such as, for example, prosthetic pulmonary and/or aortic valves.

Orthogonal or side-delivered/deliverable valves are configured to be compressed in at least one of a lateral direction (orthogonal to the blood flow direction through the valve) or an axial direction (parallel to or aligned with the blood flow direction). In some embodiments, any of the valves can be compressed in two directions—the lateral direction and the axial direction—without compressing the valve in a direction along a lengthwise or longitudinal axis of the valve (orthogonal to the blood flow direction). With orthogonal or side-delivery, the compressed valve can be inserted and/or advanced through a delivery catheter such that the central axis of the compressed valve is substantially orthogonal or perpendicular to a longitudinal or lengthwise axis of the delivery catheter. Said another way, in orthogonal or side-delivery, the lengthwise or longitudinal axis of the valve can be substantially parallel to the lengthwise or longitudinal axis of the delivery catheter through which the valve is delivered. Thus, an orthogonally delivered and/or side delivered prosthetic valve is compressed and/or delivered sideways (e.g., at a roughly 90° angle) compared to traditional processes of compressing and delivering transcatheter prosthetic valves.

In some implementations, orthogonal or side delivery can allow delivery of a larger diameter valve relative to the diameter of traditionally delivered valves. In addition, the orientation of orthogonally delivered valves relative to the annulus of the native heart valve (e.g., after being released from the delivery catheter) can allow a distal portion of the valve to be at least partially inserted into/through the annulus while the proximal portion of the valve, at least in part, remains in the delivery catheter, thereby avoiding at least some of the size constraints faced with some know traditional delivery techniques. For example, a relatively large side-deliverable prosthetic valve in an expanded configuration can have a height of about 5-60 millimeters (mm) and a diameter of about 20-80 mm, and in a compressed configuration can have a height of about 5-12 mm, a width (e.g., in a lateral direction) of about 8-12 mm, and a length (e.g., in a longitudinal or lengthwise direction) of about 25-80 mm. Moreover, orthogonal or side delivery can allow the valves to be deployed from the inferior vena cava (IVC) into the annulus of a native mitral or tricuspid valve without positioning the delivery catheter at an acute angle relative to the native valve, which is otherwise common in traditional transcatheter delivery.

Any of the prosthetic heart valves described herein can include an outer support frame that includes and/or forms a supra-annular region, a subannular region, and a transannular region coupled therebetween. The supra-annular region can form, for example, an upper collar portion of the outer support frame and can include any number of features configured to engage native tissue, an inner flow control component of the prosthetic valve, and/or a delivery, actuator, and/or retrieval mechanism. The subannular region can form, for example, one or more anchoring elements configured to engage subannular (ventricular) tissue when the prosthetic valve is seated in the native annulus. The transannular region can be coupled between the supra-annular region and the subannular region. The transannular region can form a shape such as a funnel, cylinder, flat cone, or circular hyperboloid when the outer support frame is in an expanded configuration.

In some embodiments, the outer support frame includes and/or is at least partially formed from a wire, a braided wire, or a laser-cut wire frame, and is at least partially covered with a biocompatible material. For example, the outer support frame and/or at least the transannular region thereof can include and/or form a set of compressible wire cells such as braided-wire cells, laser-cut wire cells, photolithography produced wire cells, 3D printed wire cells, wire cells formed from intermittently connected single strand wires in a wave shape, a zig-zag shape, or spiral shape, and/or combinations thereof. In some implementations the compressible wire cells can have an orientation and cell geometry substantially orthogonal to the central axis to minimize wire cell strain when the outer support frame is in a delivery configuration (e.g., a compressed, rolled, and/or folded configuration).

Any of the prosthetic heart valves described herein (and/or outer frames thereof) can include a single anchoring element or multiple anchoring elements configured to anchor the valve in the annulus of a native valve (e.g., subannular anchoring elements, supra-annular anchoring elements, and/or a combination thereof). For example, in some implementations, a prosthetic valve and/or outer frame can include one or more of a distal subannular anchoring element configured to engage ventricular tissue distal to the annulus (e.g., can extend into a right ventricular outflow tract (RVOT)); a proximal subannular anchoring element configured to engage ventricular tissue proximal to the annulus (e.g., between the septal leaflets and the posterior leaflets of the heart); a septal anchoring element configured to engage at least one of a native septal wall or a native septal leaflet when the prosthetic heart valve is seated in the annulus (e.g., to pin at least the native septal leaflet away from the coapting leaflets of the prosthetic valve); and/or any other suitable anchoring element. In some implementations, one or more of the subannular anchoring elements can stabilize the valve against at least some intra-annular rolling forces, twisting forces, and/or the like that might affect a desired location or positioning of the prosthetic valve within the annulus, (e.g., tilted, angled, twisted, rolled, etc.).

Any of the prosthetic valves and/or outer frames thereof can also include, for example, a distal and/or proximal upper anchoring element configured to be positioned into a supra-annular position in contact with and/or adjacent to supra-annular tissue of the right atrium. In some implementations, the upper anchoring element(s) can be configured to exert a force on supra-annular tissue and the lower anchoring element(s) can be configured to exert a force in an opposite direction on subannular tissue, thereby securing the prosthetic valve in the native annulus. In some implementations, the anchoring element(s) can include and/or can be formed from a wire loop or wire frame, an integrated frame section, and/or a stent, extending from the frame (e.g., about 10-40 mm away the tubular frame).

Any of the prosthetic valves described herein can include an inner flow control component that has a leaflet frame with 2-4 flexible leaflets mounted thereon. The 2-4 leaflets are configured to permit blood flow in a first direction through an inflow end of the flow control component (and/or valve) and out an outflow end of the flow control component (and/or valve), and block blood flow in a second direction, opposite the first direction. The leaflet frame can include any number of walls and/or panels of diamond-shaped or eye-shaped wire cells made from heat-set shape memory alloy material such as, for example, nickel-titanium alloys (e.g., Nitinol). The leaflet frame can be configured to be foldable along a z-axis (e.g., a longitudinal axis) from a rounded or cylindrical configuration to a flattened cylinder configuration, and compressible along a vertical y-axis (e.g., a central axis) to a compressed configuration. In some implementations, the leaflet frame can include a pair of hinge areas, fold areas, connection points, etc. that can allow the leaflet frame to be folded flat along the z-axis prior to the leaflet frame being compressed along the vertical y-axis. The leaflet frame can be, for example, a single-piece structure with two or more living hinges (e.g., stress concentration riser(s) and/or any suitable structure configured to allow for elastic/nonpermanent deformation of the leaflet frame) or a two-piece structure where the hinge areas are formed using a secondary attachment method (e.g. sutures, fabrics, molded polymer components, etc. In some embodiments, the inner flow control component in an expanded configuration forms a shape such as a funnel, cylinder, flat cone, or circular hyperboloid. In some embodiments, the inner flow control component has a leaflet frame with a side profile of a flat cone shape having an outer diameter R of about 20-60 mm, an inner diameter r of about 10-50 mm, where diameter R is great than diameter r, and a height of about 5-60 mm. In some embodiments, the leaflet frame is comprised of a wire, a braided wire, or a laser-cut wire frame.

Any of the prosthetic valves and/or components thereof may be fabricated from any suitable biocompatible material or combination of biocompatible materials. For example, an outer valve frame, an inner valve frame (e.g., of an inner flow control component), and/or components thereof may be fabricated from biocompatible metals, metal alloys, polymer coated metals, and/or the like. Suitable biocompatible metals and/or metal alloys can include stainless steel (e.g., 316 L stainless steel), cobalt chromium (Co—Cr) alloys, nickel-titanium alloys (e.g., Nitinol), and/or the like. Moreover, any of the outer or inner frames described herein can be formed from superelastic or shape-memory alloys such as nickel-titanium alloys (e.g., Nitinol). Synthetic biocompatible materials can include, for example, polyesters, polyurethanes, elastomers, thermoplastics, thermoplastic polycarbonate urethane, polyether urethane, segmented polyether urethane, silicone polyether urethane, polyetheretherketone (PEEK), silicone-polycarbonate urethane, polypropylene, polyethylene, low-density polyethylene (LDPE), high-density polyethylene (HDPE), ultra-high density polyethylene (UHDPE), polyolefins, polyethylene-glycols, polyethersulphones, polysulphones, polyvinylpyrrolidones, polyvinylchlorides, other fluoropolymers, polyesters, polyethylene-terephthalate (PET) (e.g., Dacron®), Poly-L-lactic acids (PLLA), polyglycolic acid (PGA), poly(D, L-lactide/glycolide) copolymer (PDLA), silicone polyesters, polyamides (Nylon), polytetrafluoroethylene (PTFE) (e.g., Teflon), elongated PTFE, expanded PTFE, siloxane polymers and/or oligomers, polylactones, and/or the like or block co-polymers using the same.

Any of the prosthetic valves and/or components thereof can include and/or can be formed with one or more biocompatible coating(s) and/or the like. Suitable polymer coatings can include, for example, polyethylene vinyl acetate (PEVA), poly-butyl methacrylate (PBMA), translute Styrene Isoprene Butadiene (SIBS) copolymer, polylactic acid, polyester, polylactide, D-lactic polylactic acid (DLPLA), polylactic-co-glycolic acid (PLGA), and/or the like. Some such polymer coatings may form a suitable carrier matrix for drugs such as, for example, Sirolimus, Zotarolimus, Biolimus, Novolimus, Tacrolimus, Paclitaxel, Probucol, and/or the like.

Any of the outer valve frames, inner flow control frames, and/or portions or components thereof can be internally or externally covered, partially or completely, with a natural or synthetic biocompatible and/or biological material such as pericardium, or the like. For example, where a thin, durable synthetic material is contemplated (e.g., for a covering), synthetic polymer materials such expanded PTFE, PET, or polyester (or any of the other materials described herein) may optionally be used. Suitable biological material or tissue used for coverings or the like can include, for example, chemically stabilized pericardial tissue of an animal, such as a cow (bovine pericardium), sheep (ovine pericardium), pig (porcine pericardium), or horse (equine pericardium). For example suitable tissue include, but is not limited to, tissue used in the products Duraguard®, Peri-Guard®, and Vascu-Guard®, all products currently used in surgical procedures, products which are marketed as being harvested generally from cattle less than 30 months old, and/or the like. In some implementations, a valve can be configured such that an inner surface of the outer valve frame (e.g., the wireframe cells) is covered with pericardial tissue and/or an outer surface is covered with a woven synthetic polyester material (or vice versa), or both the inner and outer surfaces are covered with pericardial tissue and the outer surface is covered with a woven synthetic polyester material.

Any of the delivery and/or retrieval systems described herein can include an outer catheter (e.g., a delivery catheter), a control catheter, and/or other suitable portion(s) that can include one or more members, components, features, and/or the like configured to facilitate delivery and/or at least partial retrieval of the valve from an annulus of a native heart valve. For example, in some implementations, a retrieval system (or at least a portion thereof) can be selectively coupled to a control catheter of a delivery/retrieval system and advanced through a delivery catheter to be place a distal end portion in an atrium of the heart near or proximate a prosthetic valve also disposed in the atrium. In some embodiments, such a retrieval system can include at least a retrieval sheath with a retrieval element advanceable through the retrieval sheath. The retrieval element can be and/or can include, for example, a self-expanding element, structure, scoop, basket, hook, and/or the like (or combinations thereof). In some implementations, the retrieval element can selectively engage a subannular portion of the prosthetic valve while the control catheter is coupled to and/or engaged with a supra-annular portion of the prosthetic valve. In some instances, the retrieval element and the control catheter can be manipulated to exert a proximally directed force on the prosthetic valve operable to pull and/or draw the prosthetic valve into the retrieval sheath. In some embodiments, the retrieval element can include one or more guide members or features configured to guide the prosthetic valve into the retrieval sheath, thereby reducing a likelihood of the valve catching, snagging, hooking, and/or becoming stuck on a distal end of the retrieval sheath.

Any method for delivering and/or deploying prosthetic heart valves described herein can include delivery of the prosthetic heart valve to a native annulus of a human heart that includes advancing a delivery catheter to at least one of (i) the tricuspid valve or pulmonary artery of the heart through the inferior vena cava (IVC) via the femoral vein or through the superior vena cava (SVC) via the jugular vein, or (ii) the mitral valve or aortic valve of the heart through a trans-atrial approach (e.g., fossa ovalis or lower), via the IVC-femoral or the SVC-jugular approach. The prosthetic valve(s) is/are removably coupled to a portion of the delivery system, placed into a compressed or delivery configuration, loaded into a delivery device and/or the delivery catheter, and advanced through a lumen of the delivery catheter. The prosthetic valve(s) can then be released from a distal end of the delivery catheter, which is disposed in an atrium of the heart using the IVC-femoral or the SVC-jugular approach. The prosthetic valve(s) is/are allowed to transition to an expanded or released configuration when released from the delivery catheter.

Any method for delivering and/or deploying prosthetic valves described herein can include positioning the valve or a portion thereof in a desired position relative to the native tissue. For example, a method can include inserting a distal subannular anchoring element of a prosthetic valve through an annulus of the native tricuspid valve and into, for example, the RVOT of the right ventricle. In some implementations, the method can include partially inserting a prosthetic valve into the annulus (e.g., of the native tricuspid valve) such that a distal portion thereof contacts native annular tissue while a proximal portion of the prosthetic valve is at least partially compressed and disposed in the delivery catheter. In some embodiments, the method can include rotating the prosthetic heart valve, using a steerable control catheter, a yoke, a set of tethers, an actuator, and/or any other portion of a delivery/retrieval system (or combinations thereof), along an axis parallel to the plane of the valve annulus. In some embodiments, the method can include transitioning one or more anchoring elements into a desired position and/or state to engage native tissue surrounding at least a portion of the annulus. In some implementations, one or more tissue anchors may be attached to the valve and to native tissue to secure the valve in a desired position.

Any method for at least partially retrieving prosthetic valves described herein can include extending a self-expanding retrieval element from a distal end of a retrieval sheath that is disposed in an atrium of a heart, wherein the retrieval element is configured to engage a portion of the prosthetic valve such as a proximal subannular anchoring element. The prosthetic valve can be pulled into the retrieval sheath to facilitate compression of the heart valve to or toward its delivery (compressed) configuration. The prosthetic valve-retrieval element combination is pulled into the retrieval catheter (e.g., using a cable, control catheter, actuator, the retrieval element, and/or any other suitable portion of a delivery and retrieval system). In some implementations, the method optionally can include pre-compressing the valve by (a) suturing a proximal subannular anchoring element against an underside of an atrial or supra-annular collar or member, or (b) pinching proximal sidewall hips of the prosthetic valve, or (c) both, prior to pulling the heart valve into the cavity of the capture element, and subsequently into the delivery and/or retrieval catheter. Similarly, in some implementations, the method optionally can include pre-compressing a connection member or yoke at a distal end of the control catheter, which in turn, can reduce a lateral extent of the connection member or yoke as well as partially folding the valve and/or otherwise biasing the valve such that that valve can be folded with an application of less external force than may otherwise be used to fold the valve.

In some implementations, placing the prosthetic valve in a compressed configuration for loading and/or advancement through a delivery sheath can include the use of, for example, one or more loading fixtures, compression devices, jigs, etc., which can, for example, reduce a force associated with compressing the valve, inserting the valve into the delivery sheath, and/or at least partially advancing the compressed valve through the delivery sheath. With retrieval, however, these devices, components, features, etc. may not be available because the valve is disposed in a chamber of a heart of a patient. Moreover, the shape and/or orientation of the valve can be preferential for delivery rather than retrieval.

Accordingly, any of the systems and/or methods for retrieving a side-deliverable prosthetic valve can include and/or can use one or more components, features, devices, etc. configured to ease retraction of the prosthetic valve into delivery and/or retrieval sheath. For example, a delivery/retrieval system can include a retrieval sheath that can be advanced over a control catheter and that replaces, for example, a delivery sheath used to deliver the prosthetic valve. In some implementations, such a retrieval sheath can have a larger diameter than the delivery sheath being replaced and thus, can provide a larger opening into which the valve is retracted. In some implementations, a delivery/retrieval system can include a retraction device or the like that can provide a mechanical advantage associated with a proximally directed force used to pull the valve into the retrieval sheath. Such a retraction device can be, for example, a ratchet mechanism or the like. In other implementations, a delivery/retrieval system can include any other suitable device, component, and/or feature that can facilitate retrieval of the valve.

The terminology used herein is for the purpose of describing particular embodiments only and is not intended to limit the full scope of the claims. Unless defined otherwise, all technical and scientific terms used herein have the same meanings as commonly understood by one of ordinary skill in the art.

As used herein, the singular forms “a,” “an,” and “the” are intended to include the plural forms as well, unless the context clearly indicates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” etc.). Similarly, the terms “comprises” and/or “comprising,” when used in this specification, specify the presence of stated features, integers (or fractions thereof), steps, operations, elements, and/or components, but do not preclude the presence or addition of one or more other features, integers (or fractions thereof), steps, operations, elements, components, and/or groups thereof. As used in this document, the term “comprising” means “including, but not limited to.”

As used herein the term “and/or” includes any and all combinations of one or more of the associated listed items. It should be understood that any suitable disjunctive word and/or phrase presenting two or more alternative terms, whether in the description, claims, or drawings, contemplate the possibilities of including one of the terms, either of the terms, or both/all terms. For example, the phrase “A or B” will be understood to include the possibilities of “A” or “B” or “A and B.”

All ranges disclosed herein also encompass any and all possible subranges and combinations of subranges thereof unless expressly stated otherwise. Any listed range should be recognized as sufficiently describing and enabling the same range being broken down into at least equal subparts unless expressly stated otherwise. As will be understood by one skilled in the art, a range includes each individual member.

The terms “prosthetic heart valve,” and/or “prosthetic valve” can refer to a combination of a frame and a leaflet or flow control structure or component, and can encompass both complete replacement of an anatomical part (e.g., a new mechanical valve that replaces a native valve), as well as medical devices that take the place of and/or assist, repair, or improve existing anatomical parts (e.g., the native valve is left in place). As used herein, the term “valve” may be used to refer to either a “prosthetic valve” or a “native valve,” and will be understood within the specific context in which the term is used.

Prosthetic valves disclosed herein can include a member (e.g., a frame) that can be seated within a native valve annulus and can be used as a mounting element for a leaflet structure, a flow control component, or a flexible reciprocating sleeve or sleeve-valve. It may or may not include such a leaflet structure or flow control component, depending on the embodiment. Such members can be referred to herein as an “annular support frame,” “tubular frame,” “wire frame,” “valve frame,” “frame,” “flange,” “collar,” and/or any other similar terms.

The term “flow control component” can refer in a non-limiting sense to a leaflet structure having 2-, 3-, 4-leaflets of flexible biocompatible material such a treated or untreated pericardium that can be sewn, joined, and/or mounted to an annular support frame, to function as a prosthetic heart valve. Such a valve can be a heart valve, such as a tricuspid, mitral, aortic, or pulmonary, that is open to blood flowing during diastole from atrium to ventricle, and that closes from systolic ventricular pressure applied to the outer surface. Repeated opening and closing in sequence can be described as “reciprocating.” The flow control component is contemplated to include a wide variety of (bio)prosthetic artificial heart valves and/or components. For example, such (bio)prosthetics can include ball valves (e.g., Starr-Edwards), bileaflet valves (St. Jude), tilting disc valves (e.g., Bjork-Shiley), stented pericardium heart-valves (bovine, porcine, ovine) (Edwards' line of bioprostheses, St. Jude prosthetic valves), as well as homograft and autograft valves. Bioprosthetic pericardial valves can include bioprosthetic aortic valves, bioprosthetic mitral valves, bioprosthetic tricuspid valves, and bioprosthetic pulmonary valves.

The terms “anchoring element” or “tab” or “arm” refer to structural elements extending from a portion of the valve or valve frame (e.g., extending away from a valve sidewall, body, or collar) to provide an anchoring or stabilizing function to the valve. When used in conjunction with the terms distal, proximal, septal, and/or anterior, it should be understood that the anchoring or stabilizing element so described is attached to and/or integral with the valve (or valve frame) at a distal, proximal, septal, and/or anterior location, respectively. A distal location on a valve refers to a portion of the valve furthest from the practitioner which exits the delivery catheter first, and which can be placed at or near distal subannular native tissue such as the ventricular outflow tract. A proximal location on a valve refers to a portion of the valve closest to the practitioner which exits the delivery catheter last, and which can be placed at or near proximal subannular native tissue such as tissue closest to the inferior vena cava. A septal location on a valve refers to a portion of the valve at a point between a proximal and a distal location, and which can be placed at or near septal subannular native tissue such as the septal leaflet or septal wall. An anterior location on a valve refers to a portion of the valve at a point between a proximal and a distal location, and which can be placed at or near anterior tissue opposite the septal tissue. When used in conjunction with the term “lower,” or “subannular” it should be understood that the anchoring or stabilizing element so described is attached to and/or integral with the valve sidewall, body, and/or frame (or portion thereof) at or along a lower or subannular region of the valve. Conversely, when used in conjunction with the term “upper,” or “supra-annular” it should be understood that the anchoring or stabilizing element so described is attached to and/or integral with the valve or frame at or along a supra-annular region, collar, or atrial cuff of the valve.

Any of the disclosed valve embodiments may be delivered by a transcatheter approach. The term “transcatheter” is used to define the process of accessing, controlling, and/or delivering a medical device or instrument within the lumen of a catheter that is deployed into a heart chamber (or other desired location in the body), as well as an item that has been delivered or controlled by such as process. Transcatheter access is known to include cardiac access via the lumen of the femoral artery and/or vein and IVC, via the lumen of the brachial artery and/or vein, via lumen of the carotid artery, via the lumen of the jugular vein and SVC, via the intercostal (rib) and/or sub-xiphoid space, and/or the like. Moreover, transcatheter cardiac access can also include a trans-atrial (e.g., fossa ovalis or lower) approach to the left atrium and/or ventricle. Transcatheter can be synonymous with transluminal and is functionally related to the term “percutaneous” as it relates to delivery of heart valves.

The mode of cardiac access can be based at least in part on a “body channel,” used to define a blood conduit or vessel within the body, and the particular application of the disclosed embodiments of prosthetic valves can determine the body channel at issue. An aortic valve replacement, for example, would be implanted in, or adjacent to, the aortic annulus. Likewise, a tricuspid or mitral valve replacement would be implanted at the tricuspid or mitral annulus, respectively. While certain features described herein may be particularly advantageous for a given implantation site, unless the combination of features is structurally impossible or excluded by claim language, any of the valve embodiments described herein could be implanted in any body channel.

The terms “expandable” and “compressible” as used herein may refer to a prosthetic heart valve or a component of the prosthetic heart valve capable of expanding and/or compressing from a first size or configuration to a second size or configuration. For example, a prosthetic valve may be “compressible” to a delivery size or configuration and “expandable” to an implantation or deployment size or configuration. An expandable/compressible structure, therefore, is not intended to refer to a structure that might undergo slight expansion/compression such as, for example, from a change in temperature or other such incidental cause, unless the context clearly indicates otherwise. Conversely, non-expandable/non-compressible should not be interpreted to mean completely rigid or a dimensionally stable, as some slight expansion/compression of conventional non-expandable/non-compressible heart valves, for example, may be observed.

The prosthetic valves disclosed herein and/or components thereof are generally capable of transitioning between two or more configurations, states, shapes, and/or arrangements. For example, prosthetic valves described herein can be compressible and/or expandable between any suitable number of configurations. Various terms can be used to describe or refer to these configurations and are not intended to be limiting unless the context clearly states otherwise. For example, a prosthetic valve can be described as being placed in a “delivery configuration,” which may be any suitable configuration that allows or enables delivery of the prosthetic valve. Examples of delivery configurations can include a compressed configuration, a folded configuration, a rolled configuration, and/or similar configuration(s) or any suitable combination(s) thereof. Similarly, a prosthetic valve can be described as being placed in an “expanded configuration,” which may be any suitable configuration that is not expressly intended for delivery of the prosthetic valve. Examples of expanded configuration can include a released configuration, a relaxed configuration, a deployed configuration, a non-delivery configuration, and/or similar configuration(s) or any suitable combination(s) thereof. Some prosthetic valves described herein and/or components or features thereof can have a number of additional configurations that can be associated with various modes, levels, states, and/or portions of actuation, deployment, engagement, etc. Examples of such configurations can include an actuated configuration, a seated configuration, a secured configuration, an engaged configuration, and/or similar configurations or any suitable combinations thereof. While specific examples are provided above, it should be understood that they are not intended to be an exhaustive list of configurations. Other configurations may be possible. Moreover, various terms can be used to describe the same or substantially similar configurations and thus, the use of particular terms is not intended to be limiting and/or to be interpreted to the exclusion of other terms unless the terms and/or configurations are mutually exclusive, or the context clearly states otherwise.

Any of the prosthetic valve embodiments described herein may be delivered via traditional transcatheter delivery techniques or via side-delivery/orthogonal-delivery techniques. For example, traditional delivery of prosthetic valves can be such that a central cylinder axis of the valve is substantially parallel to a lengthwise or longitudinal axis of a delivery catheter used to deliver the valve. Typically, the valves are compressed in a radial direction relative to the central cylinder axis and advanced through the lumen of the delivery catheter. The valves are deployed from the end of the delivery catheter and expanded outwardly in a radial direction from the central cylinder axis. The delivery orientation of the valve generally means that the valve is completely released from the delivery catheter while in the atrium of the heart and reoriented relative to the annulus, which in some instances, can limit a size of the valve.

As used herein the term “orthogonal” refers to an intersecting angle of 90 degrees between two lines or planes. As used herein, the term “substantially orthogonal” refers to an intersecting angle of 90 degrees plus or minus a suitable tolerance. For example, “substantially orthogonal” can refer to an intersecting angle ranging from 75 to 105 degrees. As used herein the terms “orthogonal delivery,” “orthogonally delivered,” “side-delivery,” “side-delivered,” and/or so forth can be used interchangeably to describe such a delivery method and/or a valve delivered using such a method. Orthogonal delivery of prosthetic valves can be such that the central cylinder axis of the valve is substantially orthogonal to the lengthwise axis of the delivery catheter. With orthogonal delivery, the valves are compressed (or otherwise reduced in size) in a direction substantially parallel to the central cylinder axis and/or in a lateral direction relative to the central cylinder axis. As such, a lengthwise axis (e.g., a longitudinal axis) of an orthogonally delivered valve is substantially parallel to the lengthwise axis of the delivery catheter. In other words, an orthogonally delivered prosthetic valve is compressed and/or delivered at a roughly 90-degree angle compared to traditional processes of compressing and delivering transcatheter prosthetic valves. Moreover, in some instances, the orientation of orthogonally delivered valves relative to the annulus can allow a distal portion of the valve to be at least partially inserted into the annulus of the native heart valve while the proximal portion of the valve, at least in part, remains in the delivery catheter, thereby avoiding at least some of the size constraints faced with some know traditional delivery techniques.

The examples and/or embodiments described herein are intended to facilitate an understanding of structures, functions, and/or aspects of the embodiments, ways in which the embodiments may be practiced, and/or to further enable those skilled in the art to practice the embodiments herein. Similarly, methods and/or ways of using the embodiments described herein are provided by way of example only and not limitation. Specific uses described herein are not provided to the exclusion of other uses unless the context expressly states otherwise. For example, any of the prosthetic valves described herein can be used to replace a native valve of a human heart including, for example, a mitral valve, a tricuspid valve, an aortic valve, and/or a pulmonary valve. While some prosthetic valves are described herein in the context of replacing a native mitral valve or a native tricuspid valve, it should be understood that such a prosthetic valve can be used to replace any native valve unless expressly stated otherwise or unless one skilled in the art would clearly recognize that one or more components and/or features would otherwise make the prosthetic valve incompatible for such use. Specific examples, embodiments, methods, and/or uses described herein should not be construed as limiting the scope of the inventions or inventive concepts herein. Rather, examples and embodiments are provided so that this disclosure will be thorough and complete, and will fully convey the scope of the inventive concepts to those skilled in the art.

The embodiments herein, and/or the various features or advantageous details thereof, are explained more fully with reference to the non-limiting embodiments that are illustrated in the accompanying drawings and detailed in the following description. Descriptions of well-known components and processing techniques may be omitted so as to not obscure the embodiments herein. Like numbers generally refer to like elements throughout. A discussion of various embodiments, components, and/or features of prosthetic valve(s) (e.g., side-deliverable, transcatheter prosthetic heart valves) is followed by a discussion of a retrieval system and methods of using such a system to optionally retrieve a prosthetic valve at least partially deployed in a heart.

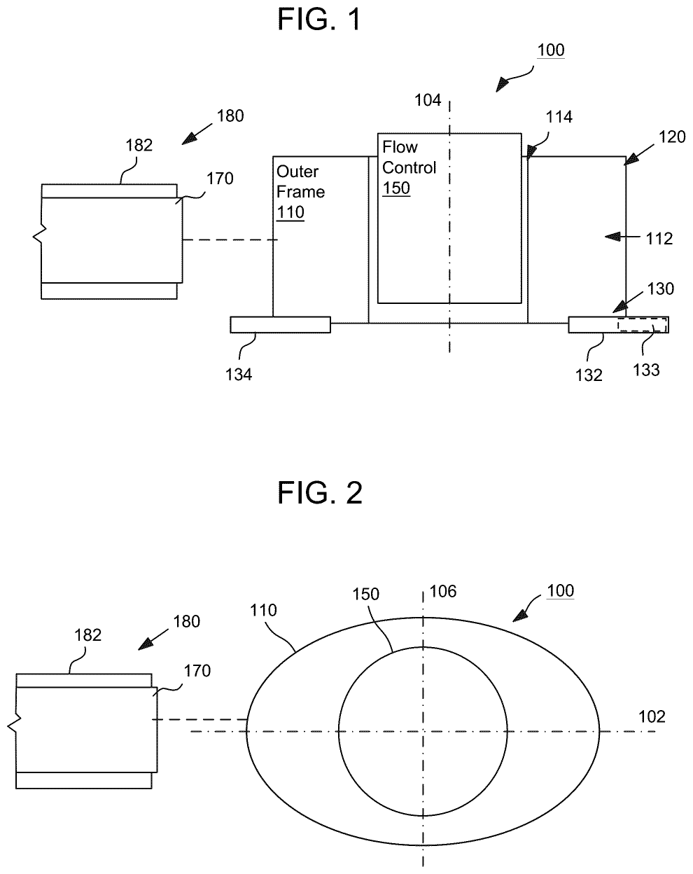

FIGS. 1-5 are various schematic illustrations of a side-deliverable transcatheter prosthetic heart valve 100 (also referred to herein as “prosthetic valve” or simply “valve”) configured to be coupled to, engaged with, and/or otherwise used with a delivery/retrieval system 180, according to an embodiment. The prosthetic valve 100 is configured to be deployed in a desired location within a body (e.g., of a human patient) and to permit blood flow in a first direction through a flow control component from an inflow end of the prosthetic valve 100 to an outflow end of the prosthetic valve 100 and to block blood flow in a second direction, opposite the first direction. For example, the prosthetic valve 100 can be configured to be deployed within the annulus of a native tricuspid valve or native mitral valve of a human heart to supplement and/or replace the functioning of the native valve. In some embodiments, the valve 100 and/or the delivery/retrieval system 180 can be similar to and/or substantially the same as the valve(s) and/or the delivery/retrieval system(s) described in WIPO Patent Publication No. WO 2021/035032 (referred to herein as “the '032 PCT”), filed Aug. 20, 2020, entitled “Delivery and Retrieval Devices and Methods for Side-Deliverable Transcatheter Prosthetic Valves,” the disclosure of which is incorporated herein by reference in its entirety.

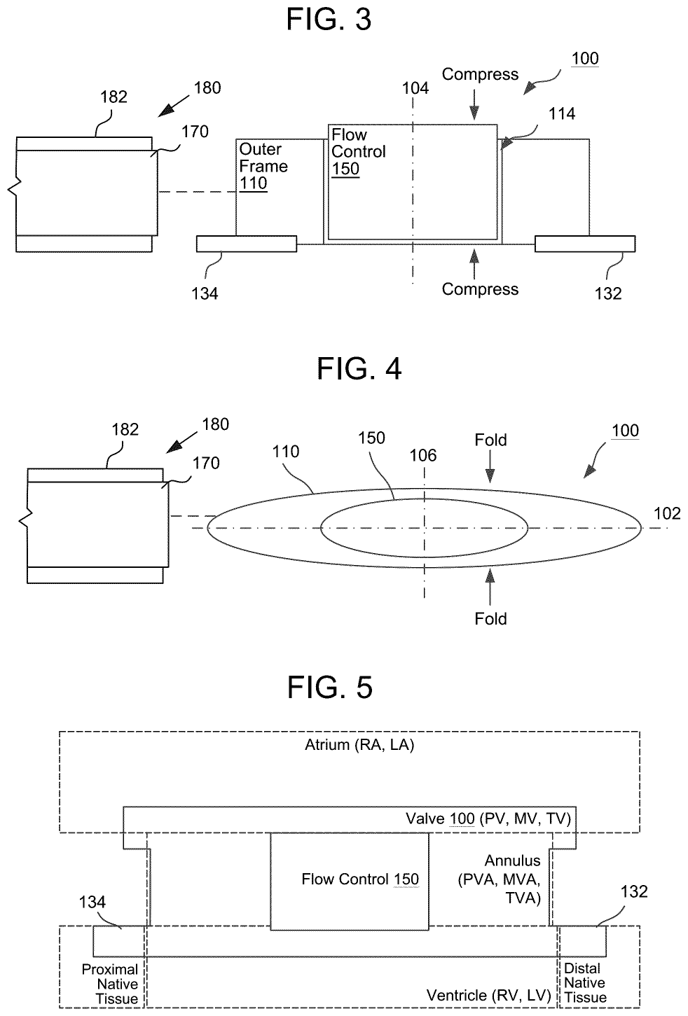

The prosthetic valve 100 is compressible and expandable between an expanded configuration (FIGS. 1 and 2) for implanting at a desired location in a body (e.g., a human heart) and a compressed or delivery configuration (FIGS. 3 and 4) for introduction into the body via, for example, a delivery catheter 182 of the delivery/retrieval system 180. For example, the prosthetic valve 100 can be compressible and expandable in at least one direction relative to a longitudinal axis 102 of the valve 100 (also referred to herein as “horizontal axis” or “lengthwise axis”). For example, the valve 100 can compressible/expandable along a central axis 104, with a first height or size along the central axis 104 when in the expanded configuration (FIG. 1) and a second height or size, less than the first height or size, along the central axis 104 when in the compressed configuration (FIG. 3). In some embodiments, the prosthetic valve 100 can be compressible and expandable in at least two directions relative to the longitudinal axis 102 of the valve 100. For example, the valve 100 can be compressible/expandable along the central axis 104 (as just described) and compressible/expandable along a lateral axis 106 that is perpendicular to both the longitudinal axis 102 and the central axis 104 (see e.g., FIGS. 1 and 2). In such embodiments, the valve 100 can have the first height and a first width when in the expanded configuration (FIGS. 1 and 2) and can have a second height and a second width—less than the first height and first width, respectively—when in the compressed configuration (FIGS. 3 and 4).

When in the expanded configuration shown in FIGS. 1, 2, and 5, the valve 100 has an extent in any direction orthogonal or lateral to the longitudinal axis 102 (e.g., along the central axis 104 and/or the lateral axis 106) that is larger than a diameter of the lumen of the delivery catheter 182 used to deliver the valve 100. For example, in some embodiments, the valve 100 can have an expanded height (e.g., along the central axis 104) of 5-60 mm. In some embodiments, the valve 100 can have an expanded length (e.g., along the longitudinal axis 102) and width (e.g., along the lateral axis 106) of about 20-80 mm, or about 40-80 mm. When in the compressed configuration shown in FIGS. 3 and 4, the valve 100 has an extent in any direction orthogonal or lateral to the longitudinal axis 102 (e.g., along the central axis 104 and/or the lateral axis 106) that is smaller than the diameter of the lumen of the delivery catheter 182, allowing the valve 100 to be delivered therethrough. For example, in some embodiments, the valve 100 can have a compressed height (e.g., along the central axis 104) and a compressed width (e.g., along the lateral axis 106) of about 6-15 mm, about 8-12 mm, or about 9-10 mm. The valve 100 can be compressed by compressing, rolling, folding, and/or any other suitable manner, or combinations thereof. In some implementations, the length of the valve 100 (e.g., along the longitudinal axis 102) is not compressed for or during delivery. Rather, in some implementations, the length of the valve 100 can be increased in response to compression of the valve 100 along the central axis 104 and/or the lateral axis 106.

In some embodiments, the valve 100 (and/or at least a portion thereof) may be heat-shaped and/or otherwise formed into any desired shape such as, for example, a roughly tubular shape, a roughly hourglass shape, and/or the like. In some embodiments, the valve 100 can include an upper atrial cuff or flange for atrial sealing, a lower ventricle cuff or flange for ventricular sealing, and a transannular section or region (e.g., a body section, a tubular section, a cylindrical section, etc.) disposed therebetween. The transannular region can have an hourglass cross-section for about 60-80% of the circumference to conform to the native annulus along the posterior and anterior annular segments while remaining substantially vertically flat along 20-40% of the annular circumference to conform to the septal annular segment. While the valve 100 is shown in FIGS. 1-5 as having a given shape, it should be understood that the size and/or shape of the valve 100 (and/or at least a portion thereof) can be based on a size and/or shape of the anatomical structures of the native tissue.

For example, the valve 100 can be centric (e.g., radially symmetrical relative to a central axis 104) or eccentric (e.g., radially asymmetrical relative to the central axis 104). In some eccentric embodiments, the valve 100, or an outer frame thereof, may have a complex shape determined by the anatomical structures where the valve 100 is being mounted. For example, in some instances, the valve 100 may be deployed in an annulus of a native tricuspid valve having a circumference in the shape of a rounded ellipse with a substantially vertical septal wall, which is known to enlarge in disease states along an anterior-posterior line. In some instances, the valve 100 may be deployed in an annulus of a native mitral valve (e.g., near the anterior leaflet) having a circumference in the shape of a rounded ellipse with a substantially vertical septal wall, which is known to enlarge in disease states. As such, the valve 100 can have a complex shape that determined, at least in part, by the native annulus and/or a disease state of the native valve. By way of example, the valve 100 or the outer frame thereof may have a D-shape (viewed from the top) so the flat portion can be matched to the anatomy in which the valve 100 will be deployed (e.g., a substantially vertical septal wall). In some embodiments, the valve 100 or the outer frame thereof can have a circumference in the shape of a rounded ellipse, such as a hyperbolic paraboloid, to account for the positions of native septal, anterior, and/or posterior leaflets, and/or the native septal wall; to avoid native electrical bundles such as the atrioventricular (A-V) node and/or A-V node-related structures like the Triangle of Koch, AV bundle, etc.; to avoid interference with coronary blood flow such as the coronary sinus; to accommodate variances in the septal wall that is known to be substantially vertical but that enlarges along the anterior-posterior axis toward the free wall in disease states; and/or the like.

As shown, the valve 100 generally includes an annular support frame 110 and a flow control component 150 mounted within the annular support frame 110. In addition, the valve 100 and/or at least the annular support frame 110 of the valve 100 can include, can couple to, and/or can otherwise engage, the delivery/retrieval system 180. The annular support frame 110 (also referred to herein as “tubular frame,” “valve frame,” “wire frame,” “outer frame,” or “frame”) can have a supra-annular region 120, a subannular region 130, and a transannular region 112, disposed and/or coupled therebetween. In some embodiments, the frame 110 can be monolithically and/or unitarily constructed. In some embodiments, one or more of the supra-annular region 120, the subannular region 130, and/or the transannular region 112 can be separate, independent, and/or modular components that are coupled to collectively form the frame 110. For example, in some embodiments, the supra-annular region 120 can be, for example, an atrial collar or cuff coupled to a top, upper, and/or supra-annular edge of the transannular region 112 and the subannular region 130 can be a bottom, lower, and/or subannular portion or section of the transannular region 112 of the frame 110.

In some implementations, a modular and/or at least partially modular configuration can allow the frame 110 to be adapted to a given size and/or shape of the anatomical structures where the valve 100 is being mounted. For example, one or more of the supra-annular region 120, the subannular region 130, and/or the transannular region 112 can be designed and/or adapted so that that the support frame 110 has any desirable height, outer diameter, and/or inner diameter such as any of those described above. Moreover, such a modular configuration can allow the frame 110 to bend, flex, compress, fold, roll, and/or otherwise reconfigure without plastic or permanent deformation thereof. For example, the frame 110 is compressible to a compressed or delivery configuration for delivery and when released it is configured to return to its original shape (uncompressed, expanded, or released configuration) substantially without plastic or permanent deformation.

The support frame 110 and/or the supra-annular region 120, subannular region 130, and/or transannular region 112 can be formed from or of any suitable material. In some embodiments, the frame 110 and/or one or more portions or regions thereof can be formed from or of a shape-memory or superelastic metal, metal alloy, plastic, and/or the like. For example, the frame 110 (e.g., one or more of the supra-annular region 120, the subannular region 130, and the transannular region 112) can be formed from or of Nitinol or the like. In some embodiments, the frame 110 (and/or any of the regions thereof) can be laser cut from a Nitinol sheet or tube. In other embodiments, the frame 110 (and/or any of the regions thereof) can be formed of or from a Nitinol wire that is bent, kink, formed, and/or manipulated into a desired shape. In still other embodiments, the frame 110 (and/or any of the regions thereof) can be formed of or from a desired material using any suitable additive or subtractive manufacturing process such as those described above. Moreover, the frame 110 and/or one or more of the supra-annular region 120, the subannular region 130, and the transannular region 112 can be formed of or from a metal or other structural frame material, which in turn, is covered by a biocompatible material such as, for example, pericardium tissue (e.g., Dura-Guard®, Peri-Guard®, Vascu-Guard®, etc.), polymers (e.g., polyester, Dacron®, etc.), and/or the like, as described above.

The supra-annular region 120 of the frame 110 can be and/or can form, for example, a cuff or collar that can be attached or coupled to an upper edge or upper portion of the transannular region 112. When the valve 100 is deployed within a human heart, the supra-annular region 120 can be an atrial collar that is shaped to conform to the native deployment location. In a tricuspid and/or mitral valve replacement, for example, the supra-annular region 120 (e.g., atrial collar) can have various portions configured to conform to the native valve and/or a portion of the atrial floor surrounding the tricuspid and/or mitral valve, respectively. In some implementations, the supra-annular region 120 can be deployed on the atrial floor to direct blood from the atrium into the flow control component 150 of the valve 100 and to seal against blood leakage (perivalvular leakage) around the frame 110.

In some embodiments, the supra-annular region 120 can be a wire frame that is laser cut out of any suitable material. In some embodiments, the supra-annular region 120 can be formed from a tube or sheet of a shape-memory or superelastic material such as, for example, Nitinol and, for example, heat-set into a desired shape and/or configuration. In some embodiments, forming the supra-annular region 120 in such a manner can allow the supra-annular region 120 to bend, flex, fold, compress, and/or otherwise reconfigure substantially without plastically deforming and/or without fatigue that may result in failure or breaking of one or more portions thereof. Moreover, the wire frame of the supra-annular region 120 can be covered by any suitable biocompatible material such as any of those described above.

The supra-annular region 120 includes a distal portion and a proximal portion. In some embodiments, the distal portion can be and/or can include a distal supra-annular anchoring element and/or the like that can engage supra-annular native tissue on a distal side of the annulus as the prosthetic valve 100 is seated into the annulus. In some embodiments, the proximal portion can be and/or can include a proximal supra-annular anchoring element and/or the like that can engage supra-annular native tissue on a proximal side of the annulus as the prosthetic valve 100 is seated in the annulus. In some embodiments, the distal portion and/or the distal supra-annular anchoring element can be sized and/or shaped to correspond to a size and/or shape of the distal portion of the atrial floor of the heart in which the prosthetic valve 100 is disposed. Similarly, the proximal portion and/or the proximal supra-annular anchoring element can be sized and/or shaped to correspond to a size and/or shape of a proximal portion of the atrial floor of the heart.

Although not shown in FIGS. 1-5, the supra-annular region 120 can be shaped and/or formed to include any number of features configured to engage native tissue and/or one or more other portions of the valve 100, the delivery/retrieval system 180, and/or the like. For example, in some embodiments, the supra-annular region 120 can include and/or can form an outer portion and an inner portion that is suspended from and/or coupled to the outer portion. In some implementations, the outer portion can be sized and/or shaped to engage native tissue, the inner portion can provide structure for mounting the flow control component 150 to the support frame 110, and one or more coverings, spacers, struts, splines, and/or structures can be disposed therebetween. In some implementations, a portion of the supra-annular region 120 can be at least temporarily coupled to and/or can at least temporarily receive a portion of the delivery/retrieval system 180, at least a portion of an actuator, at least a portion of a guidewire, and/or the like.

The transannular region 112 of the support frame 110 is coupled to the supra-annular region 120 and extends from the supra-annular region 120 and at least partially through the annulus of the native valve when the prosthetic valve 100 is seated therein. In some embodiments, the transannular region 112 can be coupled to the supra-annular region 120 such that a desired amount of movement and/or flex is allowed therebetween (e.g., welded, bonded, sewn, bound, and/or the like). For example, in some implementations, the transannular region 112 and/or portions thereof can be sewn to the supra-annular region 120 (and/or portions thereof).

The transannular region 112 can be shaped and/or formed into a ring, a cylindrical tube, a conical tube, D-shaped tube, and/or any other suitable annular shape. In some embodiments, the transannular region 112 may have a side profile of a flat-cone shape, an inverted flat-cone shape (narrower at top, wider at bottom), a concave cylinder (walls bent in), a convex cylinder (walls bulging out), an angular hourglass, a curved, graduated hourglass, a ring or cylinder having a flared top, flared bottom, or both. Moreover, the transannular region 112 can form and/or define an aperture or central channel 114 that extends along the central axis 104 (e.g., the y-axis). The central channel 114 (e.g., a central axial lumen or channel) can be sized and configured to receive the flow control component 150 across at least a portion of a diameter of the central channel 114. In some embodiments, the transannular region 112 can have a shape and/or size that is at least partially based on a size, shape, and/or configuration of the supra-annular region 120 (and/or subannular region 130) and/or the native annulus in which it is configured to be deployed. For example, the transannular region 112 can have an outer circumference surface for engaging native annular tissue that may be tensioned against an inner aspect of the native annulus to provide structural patency to a weakened native annular ring.

In some embodiments, the transannular region 112 can be a wire frame that is laser cut out of any suitable material. For example, the transannular region 112 can be formed from a tube or sheet of a shape-memory or superelastic material such as, for example, Nitinol and, for example, heat-set into a desired shape and/or configuration. Although not shown in FIGS. 1-5, in some embodiments, the transannular region 112 can include and/or can be formed with two laser cut halves that can be formed into a desired shape and/or configuration and coupled together to form the transannular region 112. The transannular region 112 can be formed to include a set of compressible wire cells having an orientation and/or cell geometry substantially orthogonal to the central axis 104 (FIG. 1) to minimize wire cell strain when the transannular region 112 is in a vertical compressed configuration, a rolled and compressed configuration, or a folded and compressed configuration. In some embodiments, forming the transannular region 112 in such a manner can allow the transannular region 112 to bend, flex, fold, deform, and/or otherwise reconfigure (substantially without plastic deformation and/or undue fatigue) in response to lateral folding along or in a direction of the lateral axis 106 (FIG. 4) and/or vertical compression along or in a direction of the central axis 104 (FIG. 3), as described in further detail herein.

As described above with reference to the supra-annular region 120, the wire frame of the transannular region 112 can be covered by any suitable biocompatible material such as any of those described above. In some implementations, the wire frame of at least the supra-annular region 120 and transannular region 112 can be flexibly coupled (e.g., sewn) to form a wire frame portion of the support frame 110, which in turn, is covered in the biocompatible material. Said another way, at least the supra-annular region 120 and the transannular region 112 can be covered with the biocompatible material prior to being coupled or after being coupled. In embodiments in which the wire frames are covered after being coupled, the biocompatible material can facilitate and/or support the coupling therebetween.

The subannular region 130 of the frame 110 can be and/or can form, for example, a cuff or collar along an end of the transannular region 112 opposite the supra-annular region 120. For example, when the valve 100 is deployed within a human heart, the subannular region 130 can be and/or can form a ventricular collar that is shaped to conform to the native deployment location. In a tricuspid and/or mitral valve replacement, for example, the subannular region 130 or collar can have various portions configured to conform to the native valve and/or a portion of the ventricular ceiling surrounding the tricuspid and/or mitral valve, respectively. In some implementations, the subannular region 130 or at least a portion thereof can engage the ventricular ceiling surrounding the native annulus to secure the valve 100 in the native annulus, to stabilize the valve 100 in the annulus, to prevent dislodging of the valve 100, to sandwich or compress the native annulus or adjacent tissue between the supra-annular region 120 and the subannular region 130 (or lower portion of the transannular region 112), and/or to seal against blood leakage (perivalvular leakage and/or regurgitation during systole) around the frame 110.

In some embodiments, the subannular region 130 is a lower or subannular portion of the transannular region 112 (e.g., the transannular region 112 and the subannular region 130 are monolithically and/or unitarily formed). Said another way, a lower or subannular portion of the transannular region 112 can form and/or include the subannular region 130. In other embodiments, the subannular region 130 is a separate and/or independent component that can be attached or coupled to a lower edge or portion of the transannular region 112, as described above with reference to the supra-annular region 120. In such embodiments, for example, the subannular region 130 can be a wire frame that is laser cut out of any suitable material such as a shape-memory or superelastic material like Nitinol, heat-set into a desired shape and/or configuration, covered by any suitable biocompatible material, and attached to a lower edge of the transannular region 112, as described above with reference to the supra-annular region 120. In some implementations, forming the subannular region 130 in such a manner can allow the subannular region 130 to bend, flex, fold, compress, and/or otherwise reconfigure substantially without plastically deforming and/or without fatigue that may result in failure or breaking of one or more portions thereof.

The subannular region 130 of the frame 110 can be shaped and/or formed to include any number of features configured to engage native tissue, one or more other portions of the valve 100, one or more portions of the delivery/retrieval system 180, one or more actuators (not shown), and/or the like. For example, as shown in FIG. 1, the subannular region 130 can include and/or can form a distal portion having a distal anchoring element 132 and a proximal portion having a proximal anchoring element 134. In some embodiments, the anchoring elements 132 and 134 are integrally and/or monolithically formed with the subannular region 130 and/or the lower or subannular portion of the transannular region 112.

In some embodiments, the distal anchoring element 132 optionally can include a guidewire coupler 133 configured to selectively engage and/or receive a portion of a guidewire or a portion of a guidewire catheter. The guidewire coupler 133 is configured to allow a portion of the guidewire or guidewire catheter to extend through an aperture of the guidewire coupler 133, thereby allowing the valve 100 to be advanced over or along the guidewire and/or guidewire catheter during delivery and deployment.

The distal anchoring element 132 is configured to engage a desired portion of the native tissue on a distal side of the native annulus to facilitate the seating, mounting, and/or deploying of the valve 100 in the annulus of the native valve. For example, in some implementations, the distal anchoring element 132 can be a projection or protrusion extending from the frame 110 (e.g., the subannular region 130 and/or the lower portion of the transannular region 112) and into a distal subannular position relative to the annulus (e.g., the RVOT for tricuspid valve replacement, and/or the like). In such implementations, the distal anchoring element 132 can be shaped and/or biased such that the distal anchoring element 132 exerts a force on the subannular tissue operable to at least partially secure, stabilize, and/or anchor the distal end portion of the valve 100 in the native annulus. In some embodiments, the distal anchoring element 132 can extend from the distal portion of the subannular region 130 (or lower portion of the transannular region 112) by about 10-40 mm.

The proximal anchoring element 134 is configured to engage subannular tissue on a proximal side of the native annulus to facilitate the seating, mounting, and/or deploying of the valve 100 in the annulus. In some embodiments, the proximal anchoring element 134 can be an anchoring element having a substantially fixed configuration. In such embodiments, the proximal anchoring element 134 can be flexible and/or movable through a relatively limited range of motion but otherwise has a single, fixed configuration. In some such embodiments, the proximal anchoring element 134 can extend from the proximal portion of the subannular region 130 (or lower portion of the transannular region 112) by about 10-40 mm.

In other embodiments, the proximal anchoring element 134 can be configured to transition, move, and/or otherwise reconfigure between two or more configurations. For example, the proximal anchoring element 134 can be transitioned between a first configuration in which the proximal anchoring element 134 extends from the subannular region 130 a first amount or distance and a second configuration in which the proximal anchoring element 134 extends from the subannular region 130 a second amount or distance, different from the first amount or distance. For example, in some embodiments, the proximal anchoring element 134 can have a first configuration in which the proximal anchoring element 134 is in a compressed, contracted, retracted, undeployed, folded, and/or restrained state (e.g., in a position that is near, adjacent to, and/or in contact with the transannular region 112 and/or the supra-annular region 120 of the frame 110), and a second configuration in which the proximal anchoring element 134 is in an expanded, extended, deployed, unfolded, and/or unrestrained state (e.g., extending away from the transannular region 112). In some implementations, the proximal anchoring element 134 in the expanded or deployed configuration (e.g., the second configuration) can extend from the transannular region 112 by about 10-40 mm and in the compressed or undeployed configuration (e.g., the first configuration) can be in contact with the transannular region 112 or can extend from the transannular region 112 by less than about 10 mm. Moreover, in some implementations, the proximal anchoring element 134 can be transitioned from the first configuration to the second configuration in response to actuation of an actuator, tensile member, portion of the delivery/retrieval system 180, and/or the like, as described in further detail herein.

In some implementations, the proximal anchoring element 134 can be transitioned from the first configuration to the second configuration during deployment to selectively engage native tissue, chordae, trabeculae, annular tissue, leaflet tissue, and/or any other anatomic structures to aid in the securement of the valve 100 in the native annulus. The proximal anchoring element 134 (and/or the distal anchoring element 132) can include any suitable feature, surface, member, etc. configured to facilitate the engagement between the proximal anchoring element 134 (and/or the distal anchoring element 132) and the native tissue. For example, in some embodiments, the proximal anchoring element 134 can include one or more features configured to engage and/or become entangled in the native tissue, chordae, trabeculae, annular tissue, leaflet tissue, and/or any other anatomic structures when in the second configuration, as described in further detail herein with reference to specific embodiments.

Although not shown in FIGS. 1-5, the subannular region 130 can include and/or form any number of additional anchoring elements such as, for example, a septal anchoring element and/or the like. For example, a septal subannular anchoring element can be configured to engage subannular septal tissue, septal leaflet tissue, and/or any other suitable tissue at, near, and/or along the septum of the heart. In some implementations, when the valve 100 is at least partially inserted into the annulus, the septal anchoring element can extend down the septal wall to pin the native septal leaflet away from, for example, the coapting leaflets of the prosthetic valve 100 and/or to stabilize the valve against any intra-annular rolling forces and/or any intra-annular twisting forces that might affect a desired location or positioning of the prosthetic valve within the annulus, (e.g., tilted, angled, twisted, rolled, etc.).

Although not shown in FIGS. 1-5, the frame 110 may also have and/or form additional functional elements (e.g., loops, anchors, etc.) for attaching accessory components such as biocompatible covers, tissue anchors, releasable deployment/retrieval controls (e.g., an actuator, a tensile member, a portion of the delivery/retrieval system 180, and/or other suitable guides, knobs, attachments, rigging, etc.) and so forth.

The flow control component 150 can refer in a non-limiting sense to a device for controlling fluid flow therethrough. In some embodiments, the flow control component 150 can be a leaflet structure having two, three, four, or more leaflets, made of flexible biocompatible material such a treated or untreated pericardium. The leaflets can be sewn or joined to a support structure such as an inner frame, which in turn, can be sewn or joined to the frame 110. The leaflets can be configured to move between an open and a closed or substantially sealed state to allow blood to flow through the flow control component 150 in a first direction through an inflow end of the valve 100 and block blood flow in a second direction, opposite to the first direction, through an outflow end of the valve 100. For example, the flow control component 150 can be configured such that the valve 100 functions, for example, as a heart valve, such as a tricuspid valve, mitral valve, aortic valve, or pulmonary valve, which can open to blood flowing during diastole from atrium to ventricle, and that can close from systolic ventricular pressure applied to the outer surface.

The inner frame and/or portions or aspects thereof can be similar in at least form and/or function to the frame 110 (e.g., outer frame) and/or portions or aspects thereof. For example, the inner frame can be a laser cut frame formed from or of a shape-memory material such as Nitinol. Moreover, the inner frame can be compressible for delivery and configured to return to its original (uncompressed) shape when released (e.g., after delivery). In some embodiments, the inner frame can include and/or can form any suitable number of compressible, elastically deformable diamond-shaped or eye-shaped wire cells, and/or the like. The wire cells can have an orientation and cell geometry substantially orthogonal to an axis of the flow control component 150 to minimize wire cell strain when the inner frame is in a compressed configuration.

In some embodiments, the flow control component 150 and/or the inner frame thereof can have a substantially cylindrical or tubular shape when the valve 100 is in the expanded configuration (see e.g., FIG. 2) and can be configured to elastically deform when the valve 100 is placed in the compressed configuration (see e.g., FIGS. 3 and 4). Although not shown in FIGS. 1-5, in some embodiments, the inner frame of the flow control component 150 can include and/or can be formed with two halves that can be coupled together to allow the inner frame to elastically deform in response to lateral compression or folding along or in a direction of the lateral axis 106 (FIG. 3), as described in further detail herein.

As shown in FIGS. 1-5, the flow control component 150 is mounted within the central channel 114 of the frame 110. More specifically, the flow control component 150 is mounted and/or coupled to the supra-annular region 120 (e.g., an inner portion thereof) and is configured to extend into and/or through the central channel 114 formed and/or defined by the transannular region 112. In some embodiments, the flow control component 150 can be coupled to the supra-annular region 120 via tissue, a biocompatible mesh, one or more woven or knitted fabrics, one or more superelastic or shape-memory alloy structures, which is sewn, sutured, and/or otherwise secured to a portion of the supra-annular region 120. In some embodiments, the flow control component 150 can be coupled to the supra-annular region 120 such that a portion of the flow control component 150 is disposed above and/or otherwise extends beyond the supra-annular region 120 (e.g., extends away from the annulus in the direction of the atrium). In some embodiments, the portion of the flow control component 150 extending above and/or beyond the supra-annular region 120 can form a ridge, ledge, wall, step-up, and/or the like. In some implementations, such an arrangement can facilitate ingrowth of native tissue over the supra-annular region 120 without occluding the flow control component 150.

The flow control component 150 can be at least partially disposed in the central channel 114 such that the axis of the flow control component 150 that extends in the direction of blood flow through the flow control component 150 is substantially parallel to the central axis 104 of the frame 110. In some embodiments, the arrangement of the support frame 110 can be such that the flow control component 150 is centered within the central channel 114. In other embodiments, the arrangement of the support frame 110 can be such that the flow control component 150 is off centered within the central channel 114. In some embodiments, the central channel 114 can have a diameter and/or perimeter that is larger than a diameter and/or perimeter of the flow control component 150. Although not shown in FIGS. 1-5, in some embodiments, the valve 100 can include a spacer or the like that can be disposed within the central channel 114 adjacent to the flow control component 150. In other embodiments, a spacer can be a cover, or the like coupled to a portion of the frame 110 and configured to cover a portion of the central channel 114. In some instances, the spacer can be used to facilitate the coupling of the flow control component 150 to the frame 110.

FIG. 5 shows the valve 100 seated in the annulus of a native heart valve after delivery and deployment of the valve 100 using the delivery/retrieval system 180. As described above, the valve 100 is compressible and expandable between the expanded configuration (FIGS. 1 and 2) and the compressed configuration (FIGS. 3 and 4). The valve 100 is in the expanded configuration prior to being loaded into the delivery/retrieval system 180 and is compressed for delivery through the delivery catheter 182. More specifically, the valve 100 is configured for transcatheter orthogonal delivery through the delivery catheter 182 to the desired location in the body, in which the valve 100 is compressed in an orthogonal or lateral direction relative to the dimensions of the valve 100 in the expanded configuration (e.g., along the central axis 104 and/or the lateral axis 106). During delivery, the longitudinal axis 102 of the valve 100 is substantially parallel to a longitudinal axis of the delivery catheter 182. In some embodiments, the devices and methods of delivering the valve 100 to the desired location in the body (e.g., via the delivery/retrieval system 180) can be similar to and/or the substantially the same as the delivery system(s) described in the '032 PCT, incorporated by reference above. Accordingly, portions and/or aspects of the devices and/or procedures used to deliver the valve 100 to, for example, the annulus of the native heart valve shown in FIG. 5 are not described in further detail herein.

As shown in FIG. 5, the valve 100 can be delivered, for example, to an atrium of the human heart (or any other space or chamber of the human heart). In some implementations, for example, the valve 100 (e.g., the supra-annular member/region 120) can be removably coupled to a control device 170 included in the delivery/retrieval system 180 that can be used to advance the compressed valve 100 through a lumen of the delivery catheter 182, as described in detail with reference to the delivery/retrieval systems in the '032 PCT. Moreover, the valve 100 can be advanced along or over a guidewire and/or guidewire catheter, through the delivery catheter 182, and into a desired position within the heart (e.g., the annulus of a native heart valve). In some embodiments, at least portion of the control device 170 or the like can extend through one or more lumens of the delivery catheter, thereby allowing a user (e.g., a doctor, surgeon, technician, etc.) to manipulate a distal end of the control device 170 and thus one or more portions of the valve 100 during deployment.

Once in the atrium and released from the delivery catheter 182, the valve 100 can transition to the expanded configuration for deployment into an annulus of a native valve such as, for example, the pulmonary valve (PV), the mitral valve (MV), the aortic valve (AV), and/or the tricuspid valve (TV). The deployment of the valve 100 can include placing the distal anchoring element 132 of the subannular region 130 in the ventricle (RV, LV) below the annulus while the remaining portions of the valve 100 are in the atrium (RA, LA). In some instances, the distal anchoring element 132 can be advanced over and/or along the guidewire or guidewire catheter (not shown) to a desired position within the ventricle such as, for example, an outflow tract of the ventricle. For example, in some implementations, the valve 100 can be delivered to the annulus of the native tricuspid valve (TV) and at least a portion of the distal anchoring element 132 can be positioned in the RVOT. In other implementations, the valve 100 can be delivered to the annulus of the native mitral valve (MV) and at least a portion of the distal anchoring element 132 can be positioned in a subannular position distal to the annulus and/or in any other suitable position in which the distal anchoring element 132 can engage native tissue, leaflets, chordae, etc.

In some implementations, the prosthetic valve 100 can be temporarily maintained in a partially deployed state. For example, the valve 100 can be partially inserted into the annulus and held at an angle relative to the annulus to allow blood to flow from the atrium to the ventricle partially through the native valve annulus around the valve 100, and partially through the valve 100, which can allow for assessment of the valve function.

As shown in FIG. 5, the valve 100 is placed or seated in the annulus (PVA, MVA, AVA, and/or TVA) of the native valve (PV, MV, AV, and/or TV) such that the subannular region 130 (e.g., a ventricular collar) is disposed in a subannular position, the transannular region 112 of the frame 110 extends through the annulus, and the supra-annular region 120 (e.g., an atrial collar) remains in a supra-annular position. In some instances, with the distal subannular anchoring element 132 in the RVOT, the delivery/retrieval system 180, the control device 170, and/or any other suitable member, tool, etc. can be used to push at least the proximal end portion of the valve 100 into the annulus, as described in detail in the '032 PCT.

In some implementations, the proximal anchoring element 134 can be maintained in its first configuration as the valve 100 is seated in the annulus. For example, as described above, the proximal anchoring element 134 can be in a compressed, contracted, and/or retracted configuration in which the proximal anchoring element 134 is in contact with, adjacent to, and/or near the transannular region 112 and/or the supra-annular region 120 of the frame 110, which in turn, can limit an overall circumference of the subannular region 130 of the frame 110, thereby allowing the subannular region 130 and the transannular region 112 of the frame 110 to be inserted into and/or through the annulus.

In some embodiments, the control device 170 of the delivery/retrieval system 180 can be configured to actuate one or more portions of the valve 100 such as, for example, the proximal anchoring element 134 between its first and second configurations. For example, the control device 170 can include one or more cables, tethers, linkages, joints, connections, tensile members, etc., that can exert a force (or can remove an exerted force) on a portion of the proximal anchoring element 134 operable to transition the proximal anchoring element 134 between the first and second configuration. For example, the subannular region 130 of the support frame 110 can be formed with the proximal anchoring element 134 biased in the uncompressed and/or expanded configuration and the control device 170 can be actuated to exert a force, via the one or more cables, tethers, etc., operable to transition the proximal anchoring element 134 to the compressed and/or retracted configuration.

In some implementations, the control device 170 can be secured and/or locked when the proximal anchoring element 134 is compressed and/or retracted (e.g., a first configuration) to at least temporarily maintain the proximal anchoring element 134 in the first configuration. As described above, in some implementations, the proximal anchoring element 134 can be in the first configuration for delivery and deployment prior to seating the valve 100 in the native annulus. Once the valve 100 is seated in the native annulus, a user can manipulate a portion of the delivery/retrieval system 180 to actuate the control device 170. In this example, actuating the control device 170 can cause the control device 170 to release and/or remove the force exerted on the proximal anchoring element 134 (e.g., via the cable(s), tether(s), etc.), thereby allowing the proximal anchoring element 134 to return to its original or biased configuration (e.g., a second configuration).

As described above, the distal anchoring element 132 can be configured to engage native tissue on a distal side of the annulus, the proximal anchoring element 134 can be configured to engage native tissue on a proximal side of the annulus (e.g., when in the second or expanded configuration), thereby securely seating the valve 100 in the native annulus, as shown in FIG. 5. In some implementations, any other or additional portions of the valve 100 can similarly engage native tissue to securely seat the valve 100 in the native annulus and/or to form a seal between the support frame 110 and the tissue forming the native annulus (e.g., an anterior anchoring element can engage subannular tissue on an anterior side of the annulus, or the supra-annular region 120 can include any number of supra-annular anchoring elements for engaging supra-annular tissue (not shown in FIGS. 1-5)). With the valve 100 secured in the annulus, the delivery/retrieval system 180 can be decoupled from the valve 100 and retracted/removed from the patient, leaving the prosthetic valve 100 in place.

While the valve 100 is described above with reference to FIG. 5 as being delivered into the heart, deployed from the delivery catheter 182, and seated/implanted into the annulus, in some instances, it may be desirable to retrieve and/or remove a side-deliverable prosthetic valve during delivery, deployment, and/or after seating the prosthetic valve in the annulus. For example, in some instances, a defect associated with the valve, a patient's condition, one or more anatomic anomalies, and/or the like may make it desirable to retrieve and/or remove a prosthetic valve from the heart. In some such implementations, a delivery/retrieval system (e.g., the delivery/retrieval system 180) can include a retrieval element and/or any additional/other components that can be used to engage the prosthetic valve and to retrieve the valve into the delivery catheter, a retrieval sheath, and/or the like.

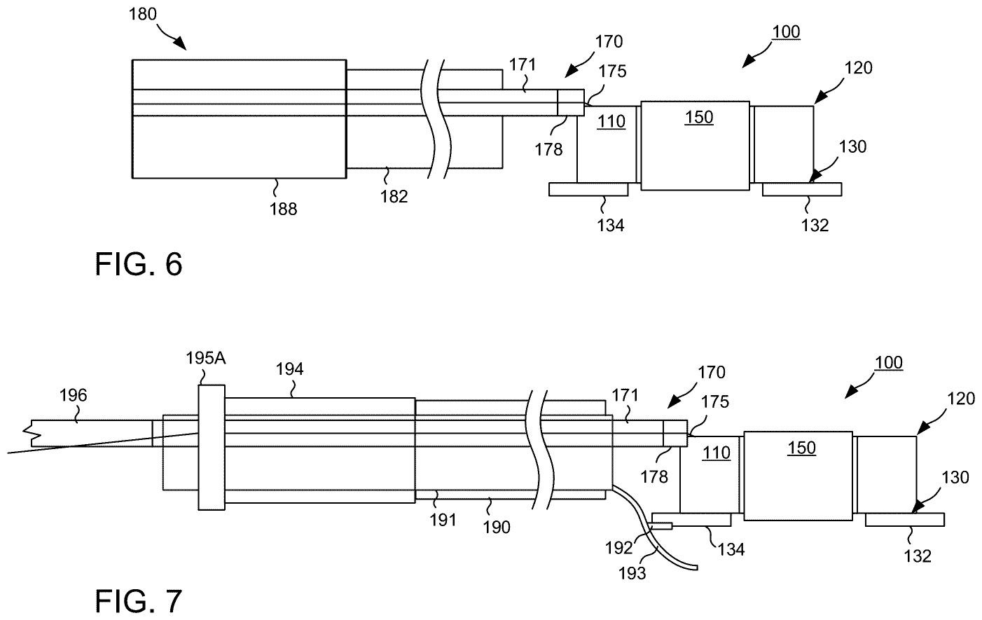

For example, FIGS. 6-9 are schematic illustrations of one or more retrieval portions, components, features, and/or aspects of the delivery/retrieval system 180 shown in FIGS. 1-5. The retrieval portions, components, etc. can be configured to allow retrieval of the prosthetic valve 100 during delivery, deployment, and/or after seating the prosthetic valve in the annulus.

FIG. 6 shows the valve 100 and a delivery portion of the delivery/retrieval system 180 used to deliver and/or deploy the valve 100 in the annulus. As described above, the delivery/retrieval system 180 includes a delivery catheter 182 configured to provide access to a chamber of the heart (e.g., via the IVC or SVC approach). A proximal end portion of the delivery catheter 182 is coupled to a handle 188 that can facilitate loading of the valve 100 and/or manipulation of the valve 100 during delivery or deployment. For example, the handle 188 can be similar to the handle of the delivery/retrieval system(s) described in the '032 PCT incorporated by reference above.

FIG. 6 further shows the control device 170 extending through the delivery catheter 182. For example, the control device 170 can include a control catheter 171 having a distal end that is coupled to and/or that includes a connection member 178 and a proximal end that is coupled to the handle 188. A set of tethers 175 can be routed through the control catheter 171 and the connection member 178, looped around a portion of the valve 100, and routed back through the connection member 178 and the control catheter 171. In some implementations, the looped arrangement of the tethers 175 removably couples the connection member 178 to, for example, the supra-annular region 120 of the valve 100, as described above. The routing of the tethers 175 can be such that the ends of each tether 175 are disposed at and/or coupled to the handle 188 to allow a user to manipulate the connection member 178 and/or the coupling between the connection member 178 and the supra-annular region 120 of the valve 100. Moreover, pulling a single end of each tether 175 in a proximal direction can remove the tethers 175 to decouple the connection member 178 from the valve 100 (e.g., after successful seating of the valve 100 in the annulus).

FIG. 6 shows the distal end of the delivery catheter 182 disposed in a chamber of the heart with the valve 100 being released or deployed outside of and/or distal to the delivery catheter 182. Accordingly, the valve 100 is in the expanded or deployed configuration. In some instances, after the valve 100 is released from the distal end of the delivery catheter 182, it may be desirable to retrieve the valve 100 (e.g., remove the valve from the body). With the valve 100 in the expanded configuration, however, retraction and/or retrieval of the valve 100 into the delivery catheter 182 requires the valve 100 to be transitioned to or toward the compressed configuration for insertion into the delivery catheter 182.

FIGS. 7-9 show a process of retrieving the valve 100 using, for example, a retrieval portion of the delivery/retrieval system 180. In some instances, the retrieval process can begin with removing one or more components included in the delivery portion of the delivery/retrieval system 180. For example, in some instances, the handle 188 can be decoupled from the delivery catheter 182, control device 170, and/or the like. In some embodiments, for example, the handle 188 can have a split body design allowing the handle to be separated into one or more pieces. Although not shown in FIG. 6, one or more tethers, tension members, actuators, etc. coupled to one or more portions of the valve 100 can also be removed. For example, the tethers used to actuate the proximal anchoring element 134 can be decoupled from the valve 100 and removed from one or more lumen of the control catheter 171. Similarly, the guidewire and guidewire catheter along which the valve 100 is advanced during the delivery can be retracted and/or removed.

In some implementations, the delivery catheter 182 also can be removed from the patient without removing the control catheter 171. For example, in implementations in which a relatively large valve is being retrieved, it may be desirable to remove the delivery catheter 182 to allow a larger diameter retrieval sheath to be advanced along the control catheter 171 to the chamber of the heart. In other implementations, the delivery catheter 182 can remain in place and retrieval components of the delivery/retrieval system 180 can retrieve the valve 100 into the lumen of the delivery catheter 182 (e.g., when retrieving relatively small valves).

While portions of the delivery/retrieval system 180 are removed, FIG. 7 shows that the control device 170 and the tethers 175 remain attached or coupled to the valve 100. For example, as with delivery and deployment, the control catheter 171 can still extend through the delivery catheter 182 with the connection member 178 at the distal end thereof still coupled to the valve 100 in the chamber of the heart. With the desired portions of the delivery/retrieval system 180 removed, the retrieval portion of the delivery/retrieval system 180 can be employed to retrieve engage and retrieve the valve 100.

FIG. 7 shows, for example, a retrieval sheath 190, a retrieval element 191, a retrieval handle 194, and an exchange catheter 196. The exchange catheter 196 is configured to couple to a proximal end of the control catheter 171. For example, in some embodiments, the distal end of the exchange catheter 196 can include a threaded male coupler that can be inserted into a lumen of the control catheter 171 to form a threaded coupling therebetween. In some embodiments, the exchange catheter 196 can, for example, extend a length of the control catheter 171 (e.g., proximally), which in turn, can allow the retrieval sheath 190 and retrieval handle 194 to be advanced over the exchange catheter 196 and at least a portion of the control catheter 171, as described in further detail herein.

As described above, the control device 170 and more specifically, the connection member 178 can remain attached to and/or in contact with the supra-annular region 120 of the valve 100 via the tethers 175. Having removed the delivery handle 188, FIG. 7 shows that the ends of each tether 175 are unattached or not anchored and can extend beyond the proximal end of the control catheter 171. In some embodiments, the exchange catheter 196 can include and/or can define one or more features that can engage and/or couple to the tethers 175 to secure the end portions thereof. For example, the exchange catheter 196 can include and/or can define a skive that can selectively receive the end portions of the tethers 175, thereby securing the end portions via a clamping force or the like. In other embodiments, the exchange catheter can include any other suitable coupler, retainer, and/or engagement feature. In this manner, control of the distal end portion of the control device 170 (e.g., the connection member 178 and the valve 100 attached thereto) can be maintained during the retrieval process.

As shown, the proximal end portion of the retrieval sheath 190 is coupled to the retrieval handle 194. The retrieval sheath 190 sheath can be, for example, a flexible catheter having any suitable size and/or diameter. In some implementations, for example, the retrieval sheath 190 and the retrieval handle 194 can replace and/or function similarly to the delivery catheter 182 and/or the delivery handle 188 (now removed). In these implementations, the retrieval sheath 190 can be a catheter having at least a larger inner diameter than the delivery catheter 182. In other implementations, the delivery catheter 182 can remain in place and the retrieval sheath 190 can be advanced through the lumen of the delivery catheter 182 (e.g., the retrieval sheath 190 has an outer diameter that is smaller than the inner diameter of the delivery catheter 182). In some instances, it may be desirable to use a retrieval sheath that is larger than the delivery catheter 182 to facilitate and/or accommodate compression of the valve 100 without a loading device.

The retrieval handle 194 is coupled to the proximal end of the retrieval sheath 190. The retrieval handle 194 can have any suitable shape, size, and/or configuration and can provide, for example, at least a portion of a user interface for the retrieval sheath 190. The proximal end portion of the retrieval handle 194 is shown having a coupler 195A configured to allow one or more devices to couple to and/or otherwise engage the retrieval handle 194. For example, in some embodiments, the coupler 195A can be a collet or the like that can be used to secure one or more devices of the delivery/retrieval system 180, as described in further detail herein.

The retrieval element 191 is shown in FIGS. 7-9 as being advanced through and/or at least partially disposed in the retrieval sheath 190. The retrieval element 191 can be any suitable device, element, member, and/or the like having any suitable size. For example, the retrieval element 191 can be and/or can include a catheter having a distal end that includes and/or is coupled to an engagement member 192 and a guide member 193. The retrieval element 191 (e.g., a retrieval catheter) can define a lumen therethrough that has a sufficiently large diameter to allow the retrieval element 191 to be disposed over and advanced along the control catheter 171, while being disposed in and/or movable through the retrieval sheath 190.

The engagement member 192 coupled to the distal end of the retrieval element 191 (e.g., and/or a retrieval catheter or catheter portion thereof) can be any suitable device, member, feature, etc. For example, in some implementations, the engagement member 192 can be a hook, a snag, a protrusion, and/or any other suitable feature. The engagement member 192 can be formed from, for example, a shape-memory material such as a super elastic metal alloy like Nitinol or the like. Accordingly, in some implementations, the engagement member 192 can be configured to transition between two or more configurations such as at least a delivery configuration and a deployment or engagement configuration. In such implementations, the engagement member 192 can have a relatively low profile when in the delivery configuration and can be allowed to transition (e.g., when extending distal to the retrieval sheath 190 and/or control catheter 171) to the deployment or engagement configuration in which the engagement member 192 forms and/or assumes a hook or other desirable shape.

The engagement member 192 is configured to engage one or more portions of the valve 100. For example, the engagement member 192 can engage, hook, snag, couple to, etc. one or more portions of the subannular region 130 of the valve 100 such as the proximal subannular anchoring element 134. More specifically, the portion of the subannular region 130 forming the proximal subannular anchoring element 134 can have and/or can form a rim (e.g., a wire rim covered in biocompatible material, cloth, etc.), which can be engaged and/or hooked by the engagement member 192, as described in further detail herein.

The guide member 193 coupled to the distal end of the retrieval element 191 (e.g., and/or a retrieval catheter or catheter portion thereof) can be any suitable device, member, feature, etc. For example, in some embodiments, the guide member 193 can be a scoop, tongue, flange, and/or any other suitable feature. In the embodiment shown in FIGS. 6-9, for example, the guide member 193 is a scoop formed out of one or more braided, mesh, and/or tube materials. In some embodiments, the braided/mesh materials can be, for example, braided tube or the like formed of a shape-memory material such as a super elastic metal alloy like Nitinol. Accordingly, in some implementations, the guide member 193 can be configured to transition between two or more configurations and/or states such as at least a delivery configuration and/or state and a deployment or guiding configuration and/or state. In such implementations, the guide member 193 can be relatively compact and/or compressed when in the delivery configuration (e.g., a compressed configuration and/or state) allowing the guide member 193 to be advanced, for example, through the retrieval sheath 190 and/or the delivery catheter 182. The guide member 193 can be allowed to expand when released from the retrieval sheath 190 and/or the delivery catheter 182 (e.g., when distal thereto) to the deployment or guiding configuration (e.g., an expanded configuration and/or state). In the deployment or guiding configuration, the braided/mesh material of the guide member 193 can expand to form the scoop or scoop-like shape. Moreover, the engagement member 192 can be coupled to, embedded in, and/or integrated with the guide member 193 such that a portion of the engagement member 192 extends outwardly from the guide member 193 (e.g., the hook or the like of the engagement member can extend outwardly from the guide member 193).

The guide member 193 is configured to guide one or more portions of the valve 100 into the retrieval sheath 190 (or delivery catheter 182). For example, during retrieval, the valve 100 can be pulled in a proximal direction toward the distal end of the retrieval sheath 190. With the valve 100 in the expanded configuration, drawing the valve 100 toward and/or into the retrieval sheath 190 (or delivery catheter 182) can begin to transition the valve 100 from the expanded configuration to the compressed configuration. As the valve 100 is being compressed, one or more portions, edges, etc. of the valve 100 may become snagged on the distal end of the retrieval sheath 190, thereby inhibiting retrieval. Accordingly, the guide member 193 in the expanded configuration can guide the one or more portions, edges, etc. of the valve 100 into the retrieval sheath 190 (or delivery catheter 182) to limit and/or substantially prevent snagging.

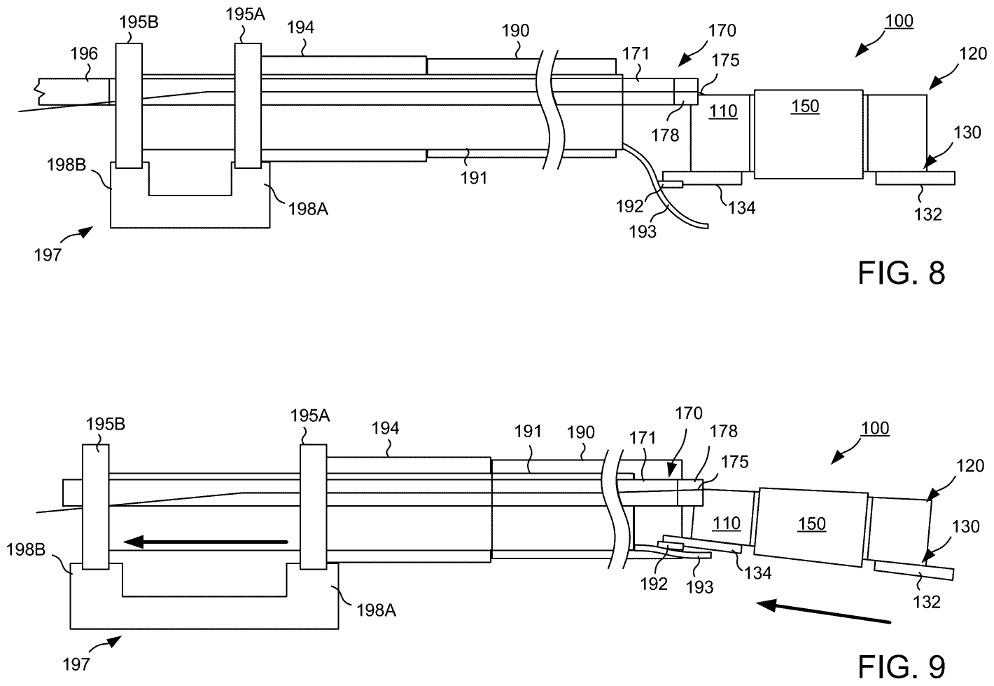

FIGS. 8 and 9 show a retractor 197 of the delivery/retrieval system 180. The retractor 197 can be any suitable shape, size, and/or configuration. For example, in some embodiments, the retractor 197 can be a ratchet mechanism and/or the like that can be manipulated to exert a force on one or more components to which it is coupled. In some embodiments, the ratchet mechanism (or other retractor) can provide a mechanical advantage that can aid in compressing the valve 100 and retracting the valve 100 into the retrieval sheath 190.

The retractor 197 includes a first end portion 198A coupled to the coupler 195A (e.g., a “first coupler”) and a second end portion 198B coupled to a second coupler 195B. As described above, the first coupler 195A is coupled to and/or included in the proximal end portion of the retrieval handle 194. The second coupler 195B, which can be similar to or substantially the same as the first coupler 195A, is at least temporarily coupled to and/or disposed about a proximal end portion of the control catheter 171. In some implementations, the first coupler 195A of the handle 194 can be in a relatively fixed or locked position relative to the delivery catheter 182, retrieval sheath 190, and/or patient. The second coupler 195B can be secured and/or coupled to the proximal end portion of the control catheter 171 and a proximal end portion of the retrieval element 191 and maintained in a fixed or locked position relative thereto.

FIG. 8 shows the retractor 197 in a first configuration in which a first distance is defined between the first end portion 198A and the second end portion 198B of the retractor 197. Accordingly, the first distance (or substantially the first distance) is defined between the first coupler 195A and the second coupler 195B. Moreover, the second coupler 195B can, for example, lock or otherwise maintain the control catheter 171 and the retrieval element 191 in a fixed relative position (i.e., relative to each other but movable relative to the retrieval sheath 190). FIG. 8 also shows that the position of the retrieval element 191 can place the engagement member 192 in contact with the proximal subannular anchoring element 134. In other words, the engagement member 192 can hook onto and/or around an edge, rim, etc. of the proximal subannular anchoring element 134.

FIG. 9 shows the retractor 197 in a second configuration in which a second distance greater than the first distance is defined between the first end portion 198A and the second end portion 198B of the retractor 197. For example, a user can engage and/or manipulate the retractor 197 to exert a force operable to move the second end portion 198B relative to the first end portion 198A. In some embodiments, the retractor 197 can be a ratchet mechanism that can be manipulated and/or actuated to increase a distance between the end portion 198A and 198B. With the couplers 195A, 195B coupled to the retrieval handle 194 and the control catheter 171 and retrieval element 191, respectively, the increase in the distance between the end portions 198A, 198B moves the distal end portions of the control catheter 171 and the retrieval element 191 relative to the retrieval sheath 190. In some implementations, the proximal movement of at least the retrieval element 191 can be such that the engagement member 192 exerts a force on the proximal subannular anchoring element 134 that can aid in transitioning the valve 100 from the expanded configuration shown in FIG. 8 to the compressed configuration.

For example, FIG. 9 shows the valve 100 being transitioned to the compressed configuration as the valve 100 is retrieved and/or retracted into the retrieval sheath 190. As described above, the arrangement of the guide member 193 can be such that the guide member 193 guides and/or directs one or more portions or edges of the valve 100 into the retrieval sheath 190. For example, the guide member 193 can be configured to guide the proximal subannular anchoring element 134 and/or an edge of the inner frame of the flow control component 150 past the distal edge of the retrieval sheath 190 and into the lumen defined by the retrieval sheath 190. Moreover, the arrangement of the retractor 197 can provide a mechanical advantage operable in generating a desired amount of force associated with and/or otherwise needed to pull the valve 100 into the retrieval sheath 190 while the valve 100 is being compressed at substantially the same time. In this manner, the delivery/retrieval system 180 can be used to retrieve a valve at least partially deployed in a chamber of a heart.

Provided below is a discussion of certain aspects or embodiments of side deliverable transcatheter prosthetic valves (e.g., prosthetic valves). The transcatheter prosthetic valves (or aspects or portions thereof) described below with respect to specific embodiments can be substantially similar in at least form and/or function to the valve 100 (or corresponding aspects or portions thereof). Thus, certain aspects and/or portions of the specific embodiments may not be described in further detail herein. A discussion of the valves and the process of delivering and/or deploying the valves using at least a delivery portion of a delivery/retrieval system is provided below followed by a discussion of using a retrieval portion of a delivery/retrieval system to retrieve, retract, and/or remove the valve from a chamber of the heart.

FIGS. 10-20 illustrate a side-deliverable (orthogonally deliverable) transcatheter prosthetic heart valve 200 (also referred to herein as “prosthetic valve” or “valve”), according to an embodiment. FIG. 10 is an illustration of a top perspective view of the valve 200. In some implementations, the valve 200 can be deployed in, for example, an annulus of a native tricuspid and/or mitral valve. The valve 200 is configured to permit blood flow in a first direction through an inflow end of the valve 200 and to block blood flow in a second direction, opposite the first direction, through an outflow end of the valve 200. For example, the valve 200 can be a side deliverable transcatheter prosthetic heart valve configured to be deployed within the annulus of a native tricuspid valve or native mitral valve of a human heart to supplement and/or replace the functioning of the native valve.

The valve 200 is compressible and expandable in at least one direction relative to an x-axis of the valve 200 (also referred to herein as “horizontal axis,” “longitudinal axis,” and/or “lengthwise axis”). The valve 200 is compressible and expandable between an expanded configuration for implanting at a desired location in a body (e.g., a human heart) and a compressed configuration for introduction into the body using a delivery catheter (not shown in FIG. 10). In some embodiments, the horizontal x-axis of the valve 200 is orthogonal to (90 degrees), or substantially orthogonal to (75-105 degrees), or substantially oblique to (45-135 degrees) to a central (vertical) y-axis when in the expanded and/or compressed configuration. Moreover, the horizontal x-axis of the valve 200 in the compressed configuration is substantially parallel to a lengthwise cylindrical axis of the delivery catheter in which the valve 200 is disposed.

In some embodiments, the valve 200 has an expanded or deployed height of about 5-60 mm, about 5-30 mm, about 5-20 mm, about 8-12 mm, or about 8-10 mm, and an expanded or deployed diameter (e.g., length and/or width) of about 25-80 mm, or about 40-80 mm. In some embodiments, the valve 200 has a compressed height (y-axis) and width (z-axis) of about 6-15 mm, about 8-12 mm, or about 9-10 mm. It some implementations, a length of the valve 200 (e.g., along the x-axis) is not compressed or otherwise reduced since it can extend along the length of the central cylindrical axis of the delivery catheter (e.g., the longitudinal or lengthwise axis).

In certain embodiments, the valve 200 can be centric or eccentric (e.g., radially symmetric or radially asymmetric, respectively, along or relative to the y-axis). In some eccentric embodiments, the frame 210 may have a D-shape in cross-section, with a flat portion or surface configured to substantially match an annulus of a native mitral valve at or near the anterior leaflet. In the example shown in FIGS. 10-20, the valve 200 is eccentric with one or more components being offset or asymmetrical region to the y-axis.

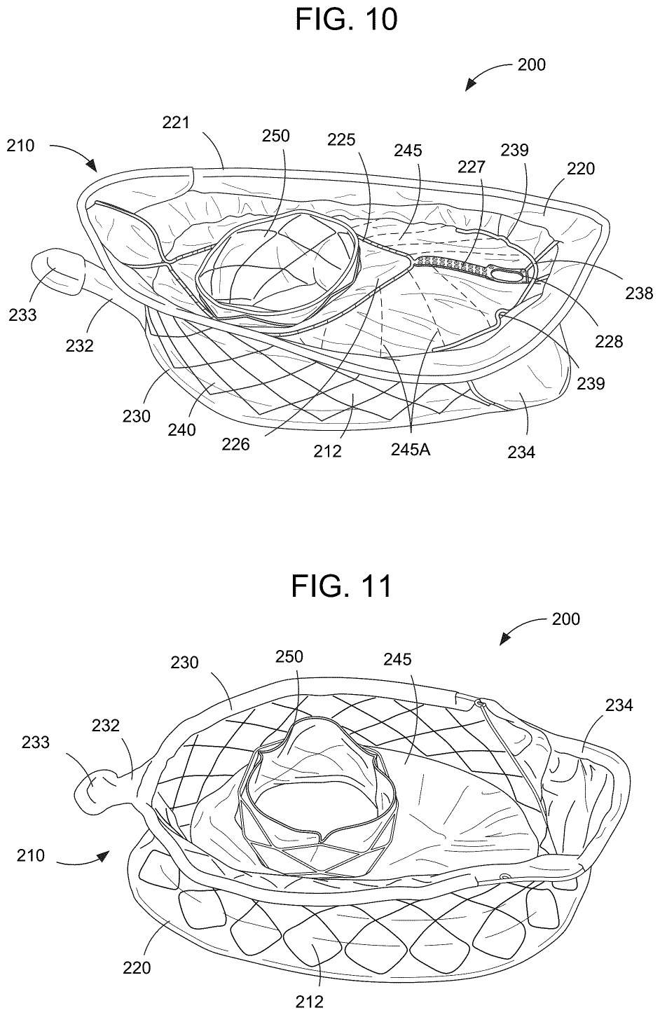

FIGS. 10 and 11 show the valve 200 including an annular outer support frame 210 and a collapsible flow control component 250 mounted within the annular outer support frame 210. The annular outer support frame 210 (also referred to herein as “outer frame”) is made from a shape-memory material such as nickel-titanium alloy (Nitinol) and is therefore a self-expanding structure from a compressed configuration to an expanded configuration. The outer frame 210 has a transannular member 212 and/or body that circumscribes, forms, and/or defines a central (interior) channel about and/or along the vertical or central axis (y-axis). The outer frame 210 has a supra-annular member 220 attached circumferentially at a top edge of the transannular member 212 and a subannular member 230 attached circumferentially at a bottom edge of the transannular member 212. As shown in FIGS. 10 and 11, at least the outer support frame 210 of the valve 200 is covered, wrapped, and/or surrounded by a biocompatible cover 240. The biocompatible cover 240 can be a mesh material, a pericardial tissue, a woven synthetic polyester material, and/or any other suitable biocompatible material such as those described above.

The biocompatible cover 240 disposed on or along the supra-annular member 220 can form a drum 245 that extends between and/or is coupled to an outer loop and an inner loop of the supra-annular member 220. As such, the drum 245 can cover a space not otherwise occupied by the flow control component 250. The drum 245 can have and/or can form a set of spokes 245A that can be used to increase a stiffness of the drum 245. The drum 245 is further shown having an attachment member 238 that can extend along or across a portion of the drum 245 (or supra-annular member 220). As described in further detail here, the attachment member 238 can facilitate a temporary and/or removable attachment to a portion of a delivery/retrieval system such as, for example, a control device, actuator, etc.

The supra-annular member 220 is shaped to conform to the native deployment location. In a tricuspid replacement, for example, the supra-annular member 220 or atrial collar can have a tall back wall portion to conform to the septal area of the native valve and can have a distal and proximal portion. The distal portion can be larger than the proximal portion to account for the larger flat space above (atrial) the ventricular outflow tract (VOT) subannular area. In a mitral replacement, for example, the supra-annular member 220 of the outer frame 210 may be D-shaped or shaped like a hyperbolic paraboloid to mimic the native structure. In some embodiments, the supra-annular member 220 of the outer frame 210 can be substantially similar in at least form and/or function to the supra-annular region 120 described above. Thus, portions and/or aspects of the supra-annular member 220 may not be described in further detail herein.

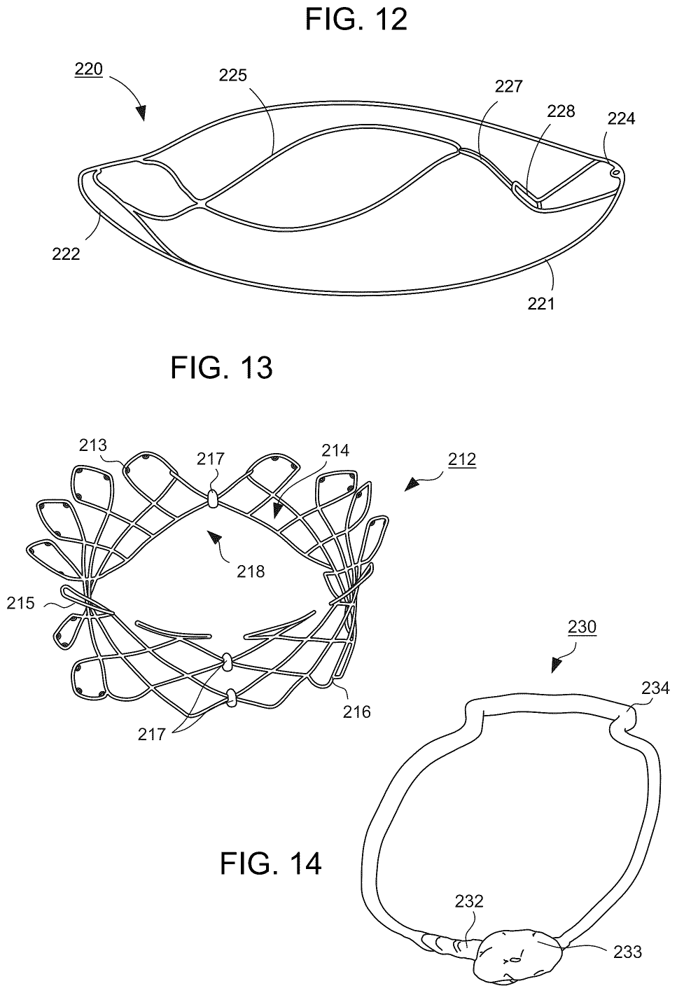

FIG. 12 shows a laser-cut wire frame portion of the supra-annular member 220 (uncovered). As shown, the supra-annular member 220 includes a distal portion 222, a proximal portion 224, an outer loop 221, an inner loop 225, and at least one spline 227. In some embodiments, the outer loop 221 can be shaped and/or sized to engage native tissue. For example, the distal portion 222 of the supra-annular member 220 (formed at least in part by the outer loop 221) is configured to engage distal supra-annular tissue and the proximal portion 224 (formed at least in part by the outer loop 221) is configured to engage proximal supra-annular tissue. The distal and proximal portions 222 and 224 can have a rounded and/or curved shape, wherein a radius of curvature of the proximal portion 224 is larger than a radius of curvature of the distal portion 222. The distal portion 222 can form, for example, a distal anchoring loop 223 that can engage distal supra-annular tissue to at least partially stabilize and/or secure the frame 210 in the native annulus. Although not shown in FIG. 12, the proximal portion 224 similarly can form a proximal upper anchoring element that can engage proximal supra-annular tissue to at least partially stabilize and/or secure the frame 210 in the native annulus.

The inner loop 225 of the supra-annular member 220 can be substantially circular, oblong, teardrop-shaped, and/or any other suitable shape. The inner loop 225 can be coupled to and/or suspended from the outer loop by the one or more splines 227. As shown in FIG. 10, the inner loop 225 can be coupled to biocompatible material 226, which can be used to couple the inner frame 251 of the flow control component 250 to the inner loop 225 of the support frame 210. In some implementations, suspending the inner loop 225 from the outer loop 221 can, for example, at least partially isolate the inner loop 225 (and the flow control component 250 coupled to the inner loop 225) from at least a portion of the force associated with transitioning the frame 210 between the expanded configuration and the compressed configuration, as described above with reference to the frame 210.

The one or more splines 227 of the supra-annular member 220 can be any suitable shape, size, and/or configuration. For example, in some embodiments, the supra-annular member 220 can include a proximal spline 227 and one or more distal splines 227. The distal splines 227 can couple a distal portion of the inner loop 225 to a distal portion of the outer loop 221. Similarly, the proximal spline 227 can couple a proximal portion of the inner loop 225 to a proximal portion of the outer loop 221. In some embodiments, the proximal spline 227 can be configured to receive, couple to, and/or otherwise engage an actuator, a control device, and/or a portion of a delivery system. For example, the proximal spline 227 includes, forms, and/or can be coupled to a waypoint 228 that can be used to couple and/or to receive one or more portions of the control device and/or delivery system, as described above with reference to the frame 110.

As shown in FIGS. 10-12, in this embodiment, the supra-annular member 220 has a bowed configuration in which the spline 227 protrudes away from other portions of the supra-annular member 220. For example, the laser cut frame of the supra-annular member 220 can be formed with the spline 227 having the bowed configuration (FIG. 12). In some implementations, bowed spline 227 can exert a force on the drum 245 that bows the drum 245 and increases a tension across the area of the drum 245. The increase in tension, alone or in conjunction with the spokes 245A, increases a relative stiffness of the drum 245, which can reduce and/or limit an amount of drum deformation during, for example, diastole or systole, thereby enhancing performance of the valve 200 and/or reduce fatigue in or along the drum 245. Said another way, the pressure produced on the atrial side of the drum 245 during contraction of the atrium (diastole) is not sufficient to invert the bowed configuration of the drum 245 (e.g., will not produce an oil-can like deflection) due to the bowed spline 227. The bowed configuration of the drum 245 can also withstand the greater pressure produced on the ventricle side of the drum 245 during contraction of the ventricle (systole) without substantial deflection. Moreover, the bow in the spline 227 can be such that the waypoint 228 is positioned at a desired angle and/or orientation to facilitate the insertion or retrieval of one or more portions of the delivery system through the waypoint 228.

FIG. 13 is a distal perspective view illustrating the transannular member 212 of the outer frame 210 of the valve 200. In some embodiments, the transannular member 212 of the outer frame 210 can be substantially similar in at least form and/or function to the transannular region 112 (and/or member) described above. Thus, portions and/or aspects of the transannular member 212 may not be described in further detail herein.

The transannular member 212 can be shaped and/or formed into a ring, a cylindrical tube, a conical tube, and/or any other suitable annular shape. In some embodiments, the transannular member 212 may have a side profile of a concave cylinder (walls bent in), an angular hourglass, a curved, graduated hourglass, a ring or cylinder having a flared top, flared bottom, or both. Moreover, the transannular member 212 can form and/or define an aperture or central channel 214 that extends along the central axis 204 (e.g., the y-axis). The central channel 214 (e.g., a central axial lumen or channel) can be sized and configured to receive the flow control component 250 across a portion of a diameter of the central channel 214. In some embodiments, the transannular member 212 can have a shape and/or size that is at least partially based on a size, shape, and/or configuration of the supra-annular member 220 and/or subannular member 230 of the support frame 210, and/or the native annulus in which it is configured to be deployed, as described above.

The transannular member 212 can be and/or can include a wire frame that is laser cut out of Nitinol or the like and, for example, heat-set into a desired shape and/or configuration. The transannular member 212 can be formed to include a set of compressible wire cells 213 having an orientation and/or cell geometry substantially orthogonal to the central axis extending through the central channel 214 to minimize wire cell strain when the transannular member 212 is in a vertical compressed configuration, a rolled and compressed configuration, or a folded and compressed configuration. As shown in FIG. 13, the transannular member 212 includes a first laser-cut half 215 (e.g., an anterior side) and a second laser-cut half 216 (e.g., a posterior side) that can be formed into a desired shape and coupled together to form the transannular member 212. The first laser-cut half 215 (e.g., the anterior side) and the second laser-cut half 216 (e.g., the posterior side) can be coupled at one or more hinge points 217 along a distal portion and a proximal portion of the transannular member 212. More specifically, the first laser-cut half 215 (e.g., the anterior side) and the second laser-cut half 216 (e.g., the posterior side) can be coupled along the distal side of the transannular member 212 via two sutures forming two hinge or coupling points 217 and can be coupled along the proximal side of the transannular member 212 via one suture forming a single hinge or coupling point 217.

In some embodiments, forming the transannular member 212 in such a manner can allow the transannular member 212 to bend, flex, fold, deform, and/or otherwise reconfigure (substantially without plastic deformation and/or undue fatigue) in response to lateral folding along or in a direction of a lateral or z-axis and/or vertical compression along or in a direction of the central or y-axis. Moreover, coupling at the hinge points 217 using sutures can allow for a desired amount of slippage between the sutures and the anterior/posterior sides 215/216, which in turn, can limit and/or substantially prevent binding, sticking, and/or failure in response to folding along the lateral or z-axis.

As shown in FIG. 13, the proximal portion of the transannular member 212 includes a single hinge or coupling point 217. In some embodiments, the transannular member 212 can define a gap or space 218 below the proximal hinge or coupling point 217 that can provide space to allow a proximal anchoring element of the subannular member 230 to transition between a first configuration and a second configuration, as described in further detail herein.

FIG. 14 is a distal perspective view illustrating the subannular member 230 of the outer frame 210 of the valve 200. In some embodiments, the subannular member 230 of the frame 210 can be similar in at least form and/or function to the subannular region 130 described above. Thus, portions and/or aspects of the subannular member 230 may not be described in further detail herein.

As shown, the subannular member 230 of the frame 210 includes and/or forms a distal portion having a distal anchoring element 232 and a proximal portion having a proximal anchoring element 234. The anchoring elements 232 and 234 are integrally and/or monolithically formed with the subannular member 230. The distal anchoring element 232 and the proximal anchoring element 234 of the subannular member 230 can be any suitable shape, size, and/or configuration. The distal anchoring element 232 is shown as including an atraumatic end that forms a guidewire coupler 233 configured to selectively engage and/or receive a portion of a guidewire catheter 284 (having a guidewire disposed therein) through an opening, hole, aperture, port, etc., defined by the guidewire coupler 233 (see e.g., FIGS. 18-20). With the guidewire catheter 284 extending through the guidewire coupler 233, the valve 200 is allowed to be advanced over or along a placed guidewire disposed in the guidewire catheter 284. In some implementations, the guidewire catheter 284 can extend below the valve 200 and beyond the distal anchoring element 232, and can provide a desired stiffness during delivery and/or deployment.

The anchoring elements 232 and/or 234 are configured to engage a desired portion of the native tissue to mount the frame 210 to the annulus of the native valve in which it is deployed. For example, the distal anchoring element 232 can extend (e.g., about 10-40 mm) from the subannular member 230 and into a RVOT or other ventricular position. The distal anchoring element 232 can be shaped and/or biased such that the distal anchoring element 232 exerts a force on the subannular tissue operable to at least partially secure the distal end portion of the frame 210 in the native annulus.

The proximal anchoring element 234 can be configured to engage subannular tissue on a proximal side of the native annulus to aid in the securement of the frame 210 in the annulus. As described above, the subannular member 230 of the frame 210 can be and/or can include, for example, a laser cut wire frame formed of a shape-memory material such as Nitinol, which is heat-set into a desired shape and wrapped in a biocompatible material (e.g., a fabric and/or the like). The proximal anchoring element 234 is configured to transition, move, and/or otherwise reconfigure between a first configuration in which the proximal anchoring element 234 extends from the subannular member 230 a first amount or distance and a second configuration in which the proximal anchoring element 234 extends from the subannular member 230 a second amount or distance. Said another way, the proximal anchoring element 234 can be, for example, a movable anchoring element configured to be moved and/or otherwise transitioned (e.g., by an actuator) between a first configuration and a second configuration to reduce a perimeter of the subannular member 230 during delivery and/or deployment.

As described above, the proximal anchoring element 234 can be in a compressed, contracted, retracted, undeployed, folded, and/or restrained state (e.g., a position that is near, adjacent to, and/or in contact with the transannular member 212 and/or the supra-annular member 220 of the support frame 210) when in the first configuration, and can be in an expanded, extended, deployed, unfolded, and/or unrestrained state (e.g., extending away from the transannular member 212) when in the second state. In some embodiments, the proximal anchoring element 234 can be biased and/or heat-set in the second configuration. Moreover, in some implementations, the space 218 defined by the transannular member 212 of the outer frame 210 is configured to provide sufficient room to allow the proximal anchoring element 234 to transition between the first and second configurations.

The proximal anchoring element 234 can be configured to move in any suitable direction from the first, extended configuration to the second, compressed configuration based at least in part on how the proximal anchoring element 234 is coupled to an actuator and/or the like. For example, the proximal anchoring element 234 can be moved inward toward the inner flow control component 250, moved upward toward the supra-annular member 220 and/or portion thereof, and/or moved toward an anterior side or a posterior side of the valve 200. Moreover, with the transannular member 212 of the frame 210 coupled to the subannular member 230, actuation of an actuator, control device, etc., can, in some instances, move one or more portions of the transannular member 212, as described in further detail herein.

The collapsible (inner) flow control component 250 is mounted within the outer frame 210. The flow control component 250 has a foldable and compressible inner wire frame 35 (also referred to as “inner leaflet frame” or “inner frame”) with two (or more) fold areas, hinge areas, coupling areas, elastically deformable regions, etc. A set of 2-4 flexible leaflets 256 are mounted in or on the inner frame 251 (not shown in FIG. 10). In some embodiments, the flow control component 250 has three (3) flexible leaflets 256 (e.g., cusps or pockets) mounted within the inner frame 251, as described in further detail herein.

The inner flow control component 250, like the outer frame 210, is foldable and compressible. For example, the inner frame 251 is foldable along or in the direction of a z-axis (e.g., foldable at the fold areas or the like) from a cylindrical configuration to a flattened cylinder configuration (or a two-layer band), where the fold areas are located on a distal side and on a proximal side of the inner frame 251. The flow control component 250, like the outer frame 210, is also vertically (y-axis) compressible to a shortened or compressed configuration. By folding (compressing) in the direction of the z-axis and vertically compressing in the y-axis, the valve 200 is permitted to maintain a relatively large dimension along the horizontal (x-axis). In some implementations, the outer frame 210 and the flow control component 250 are reduced along z-axis until the side walls are in contact or nearly so. This also allows the outer frame 210 and the flow control component 250 to maintain the radius along the horizontal axis (x-axis), to minimize the number of wire cells that can be damaged by forces applied during folding and/or compression when loading the valve 200 into the delivery catheter.

The flow control component 250 has a diameter and/or perimeter that is smaller than a diameter and/or perimeter of the central channel of the outer frame 210. The flow control component 250 is mounted to or within the outer frame 210 such that a central or vertical axis (y-axis) of the inner frame 251 is parallel to the central or vertical axis (y-axis) of the outer frame 210. In some embodiments, the y-axis defined by the inner frame 251 is parallel to but offset from the y-axis defined by the outer frame 210 (FIG. 10). In some implementations, a spacer element (e.g., the drum 245 or a portion thereof) is disposed within and/or across the central channel and can facilitate the mounting of a portion of the flow control component 250 (e.g., an otherwise unsupported portion) to the outer support frame 210 and/or an ingrowth of native tissue over at least a portion of the supra-annular member 220 of the valve 200.

In certain embodiments, the inner frame 251 can have a diameter of about 25-30 mm, the outer frame 210 (or the transannular member 212 thereof) can have a diameter of about 50-80 mm, and the supra-annular member 220 (or atrial collar) extend beyond the top edge of the transannular member 212 by about 20-30 mm to provide a seal on the atrial floor against perivalvular leaks (PVLs). The flow control component 250 and the outer frame 210 can be foldable (e.g., in the direction of the z-axis) and/or compressible (e.g., in the direction of the y-axis) to reduce a size of the entire valve 200 to fit within the inner diameter of a 24-36 Fr (8-12 mm inner diameter) delivery catheter (not shown in this FIG. 10).

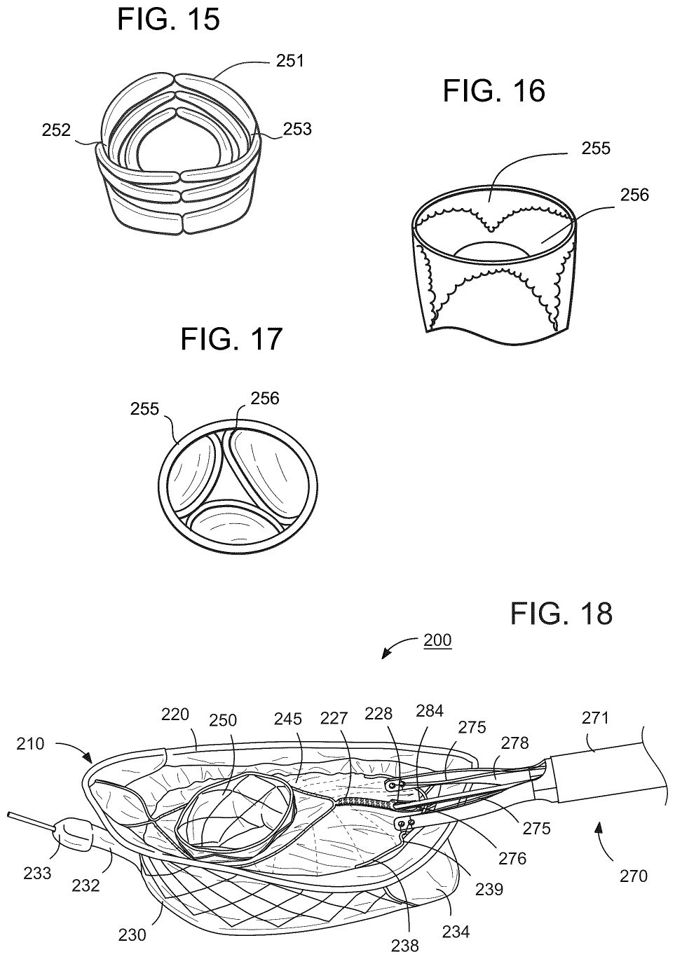

FIGS. 15-19 illustrate at least a portion of the flow control component 250 included in the valve 200. For example, FIG. 15 is an illustration of a top perspective view of the inner leaflet frame 251. In some embodiments, the inner leaflet frame 251 is formed of two separate wireframe sheets or members that are coupled at lateral connection points 252, 253 (e.g., fold areas, elastically deformable regions, coupled edged portions, etc.). The inner leaflet frame 251 is shown in an expanded or cylindrical configuration (e.g., prior to being folded and/or compressed).

Although not shown, the inner leaflet frame 251 can be transitioned from the expanded or cylindrical configuration to an at least partially folded configuration. The inner leaflet frame 251 can have wireframe sidewalls that allow for rotating or hinging at least at the lateral connection points 252, 253. The inner leaflet frame 251 can be configured to fold in response to the valve 200 being folded and/or compressed for delivery. When transitioned, for example, to a completely folded configuration, the wireframe sidewalls can be rotated, hinged, and/or folded at their lateral connection points 252, 253. In addition, the inner leaflet frame 251 can be vertically compressed into a compressed configuration. The wireframe sidewalls can form cells (e.g., diamond-shaped cells or the like) that can be oriented in a direction of compression to allow for elastic compression of the inner frame 251. In some embodiments, the inner frame 251 can be vertically compressed into a pleated or accordion (compressed) configuration.

In some embodiments, the inner leaflet frame 251 of the flow control component 250 can be formed from a linear wireframe or laser cut sheet prior to being further assembled into a cylinder structure (e.g., as shown in FIG. 15). The inner leaflet frame 251 can be formed into the cylinder structure or configuration (or a conical structure or configuration) with edge portions of the linear wireframe sheet being connected or coupled at the lateral connection points 252, 253 (e.g., hinge areas, fold areas, etc.). Moreover, the inner leaflet frame 251 can be expanded (e.g., driven, formed, bent, etc.) from the linear sheet configuration into the cylinder structure or configuration.

FIGS. 16 and 17 illustrate a structural band 255 of pericardial tissue with the flexible leaflets 256 (e.g., cusps or pockets) sewn into the structural band 255. FIGS. 16 and 17 are a side perspective view and a bottom view, respectively, illustrating the structural band 255 and flexible leaflets 256 before assembly and/or mounting on and/or into the inner frame 251 to form the collapsible (foldable, compressible) flow control component 250. FIG. 16 shows the structural band 255 formed of pericardial tissue with the flexible leaflets 256 sewn into the structural band 255, after assembly into the cylindrical leaflet configuration, the flexible leaflets 256 being disposed on an inner surface of the structural band 255. The flexible leaflets 256 can be sewn into the structural band 255 such that an open edge extends outward, and a sewn edge forms a closed top parabolic edge providing attachment. FIG. 17 is an illustration of a bottom view of the flow control component 250. The cylindrical structural band 255 and flexible leaflets 256 are shown with partial coaptation towards forming a closed fluid-seal. Although not show, the cylindrical structural band 255 can be mounted to or in the inner leaflet frame 251 (FIG. 15) to collectively form the flow control component 250, which in turn, is mounted to the inner loop 225 of the supra-annular member 220 of the outer support frame 210, as described in detail above with reference to FIGS. 10 and 11.

FIGS. 18-20 are various views showing the prosthetic valve 200 removably coupled to a control device 270 used to advance and/or retract the valve through a delivery catheter and/or to actuate one or more portions of the valve 200 such as at least the subannular member 230 of the valve frame 210, as described herein. FIG. 18 is an elevated side perspective view the prosthetic valve 200 removably coupled to the control device 270. The control device 270 and/or at least a portion thereof includes a control catheter 271 with a connection member 278 coupled to and/or disposed at a distal end. The control catheter 271 can be, for example, a multi-lumen steerable catheter, having one or more components of the control device 270 extending therethrough, as described in detail in the '032 PCT incorporated by reference above. The connection member 278 is removably coupleable to the supra-annular member 220 of the valve frame 210 and thus, connects the valve 200 to the control catheter 271. As described in further detail herein, the control catheter 271 can be manipulated to, for example, advance the prosthetic valve 200 through a delivery catheter (not shown), control or steer the prosthetic valve 200 during deployment, retrieve and/or withdraw the prosthetic valve 200 into the delivery catheter (e.g., after at least partial deployment), and/or the like.

FIG. 18 shows the connection member 278 having a wishbone or yoke configuration, though other configurations are possible. As such, the connection member 278 can have a first portion, side, and/or arm and a second portion, size, and/or arm opposite the first portion, side, and/or arm. The connection member 278 can be configured to transition between an expanded configuration and a compressed configuration to, for example, allow the control catheter 271 (and the connection member 278 disposed at the distal end thereof) to be advanced through a delivery catheter. The connection member 278 can be formed from any suitable material such as a shape-memory allow like Nitinol or the like.

In some embodiments, the connection member 278 can be in contact with and/or removably coupled to the drum 245 of the supra-annular member 220 and/or any other suitable portion of the frame 210 or valve 200. The connection member 278 can removably couple to the valve 200 via sutures, tethers, cables, clips, couplers, and/or any other removable coupling. For example, in some embodiments, the control device 270 can include a set of tethers 275 extending from one or more lumen defined by the control catheter 271. The tethers 275 are shown extending from the control catheter 271, looping through a set of openings defined along or by each side or arm of the connection member 278 (yoke), looping around one or more attachment members 238 of the valve 200, and extending back into the corresponding lumen of the control catheter 271. The attachment member(s) 238 can be formed by, coupled to, and/or extend from the supra-annular member 220 (e.g., the drum 245). In some embodiments, the attachment member 238 of the valve 200 can be a tether, suture, cable, frame structure, and/or the like that can be coupled to and/or extend from a wire frame portion of the supra-annular member 220 or, for example, the drum 245 (or other biocompatible covering). Moreover, the attachment member 238 can form a pair of loops 239 or the like around which the tethers 275 of the control device 270 can be routed or looped.

The looped arrangement of the tethers 275 through and/or around the connection member 278 and the attachment member 238 of the valve 200 is such that each of the proximal end and the distal end of the tether 275 extends through and outside of (e.g., proximal to) a single control arm 277 of the control portion 272. As such, a proximally directed force can be exerted on each of the proximal end and the distal end of the tether(s) 275 to increase a tension along the tether 275, which pulls the connection member 238 toward the drum 245, thereby securing the connection member 278 to the valve. Conversely, a proximally directed force exerted on only one of the proximal end or the distal end of the tether(s) 275 can disengage the tether(s) 275 from the connection member 278 and can withdraw the tether(s) 275 from the control device 270, which in turn, can allow the connection member 278 to be decoupled or removed from the valve 200.

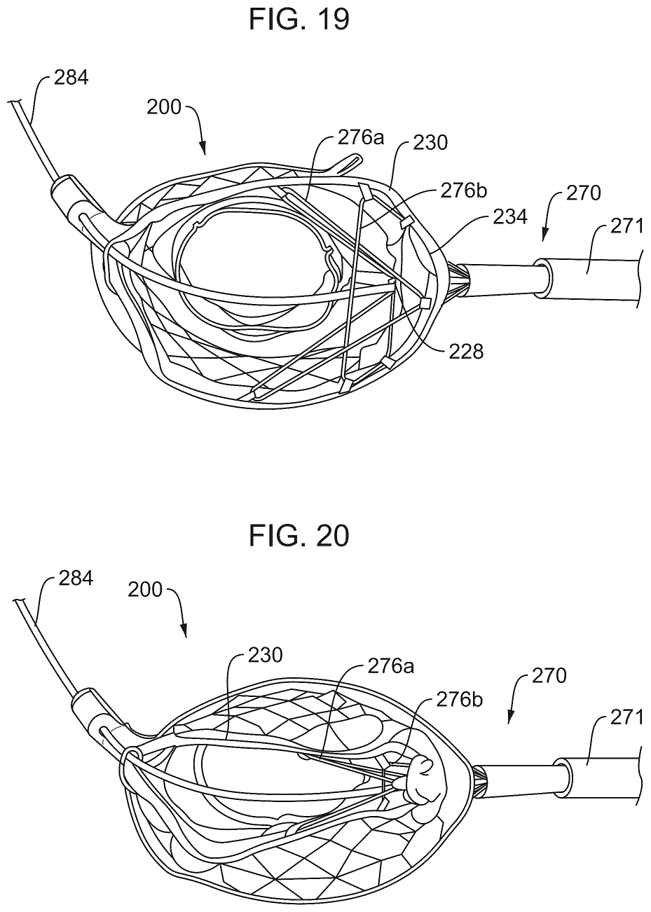

FIG. 18 further shows the guidewire catheter 284 of the delivery system extending through, for example, the waypoint 228 or opening in the supra-annular member 230 and/or drum 245 thereof and extending through the guidewire coupler 233 of the distal anchoring element 232. As shown in FIGS. 19 and 20, the guidewire catheter 284 can extend below the flow control component 250 of the valve 200. Prior to and/or as a part of delivery, the guidewire catheter 284 can be advanced and/or inserted through the valve 200 and advanced over a guidewire already placed in a desired position within the heart. As such, delivering the valve 200 in a compressed configuration through a delivery catheter includes advancing the guidewire catheter 284 along the guidewire. The guidewire catheter 284 can extend through and beyond the guidewire coupler 233 of the distal anchoring element 232 (e.g., a distal end of the guidewire catheter 284 can be distal to the guidewire coupler by about 0.1 centimeter (cm) to about 1.0 cm, or more).

The guidewire catheter 284 can be sufficiently stiff to, for example, limit and/or define (at least in part) a range of motion of the valve 200 during delivery. For example, the guidewire catheter 284 can define an axis about which the valve 200 can rotate during delivery but can substantially limit or oppose movement of the valve 200 in other directions. In some implementations, the arrangement of the connection member 278 (e.g., yoke) and the guidewire catheter 284 can allow for greater control of a position of the valve 200 during delivery. The guidewire catheter 284 and/or one or more portions of the valve 200 (e.g., the subannular member 230) can also include radiopaque markers allowing for enhanced visualization during image guided delivery. For example, in some instances, a radiopaque marker or wire can be placed relative to an annular plane of the native valve and can define a landmark during image guided delivery. In such instances, the radiopaque markers on the guidewire catheter 284 and/or other portion(s) of the valve 200 (e.g., the subannular member 230) can be used to align, orient, locate, index, etc. the valve 200 relative to the landmark, which in turn, corresponds to the annular plane of the native valve. Thus, image guided delivery can allow a user to visualize the valve 200 during delivery and/or deployment and can allow the user to visualize when the valve 200 has been seated in the annulus (e.g., the radiopaque marker bands of the valve 200 are below or in a subannular direction relative to the radiopaque landmark.

FIG. 18 further shows tethers 276A and 275B (e.g., tethers, sutures, cables, tensile members, and/or the like) extending from the control catheter 271 (e.g., through one or more lumen thereof) and through the waypoint 228. The control device 270 can include a single tether or multiple tethers (e.g., one tether, two tethers, three tethers, four tethers, five tethers, six tethers, seven tethers, eight tethers, nine tethers, ten tethers, or more, each of which can be removably coupled to one or more attachment points on the valve 200). In this embodiment, for example, the control device 270 includes two tethers 276A and 276B. The tethers 276A and 276B can be, for example, configured to actuate and/or transition one or more portions of the valve 200 such as the subannular member 230 and/or at least the proximal anchoring element 234 thereof.

FIGS. 19 and 20 are bottom views of the valve 200 showing the tethers 276A and 276B extending from the control catheter 271 and through the waypoint 228 of the supra-annular member 220 to removably attach to the subannular member 230 and/or the proximal subannular anchoring element 234. The tethers 276A and 276B can be looped around and/or through attachment points along the subannular member 230, and then can be routed back through the waypoint 228 and the control catheter 271 such that both ends of each tether 276A and 276B are outside the patient, thereby allowing manipulation of the tethers 276A and 276B to actuate the valve 200 and/or to transition a shape of the proximal anchoring element 234, the subannular member 230, and/or other portions of the valve 200 to facilitate seating at least a proximal side of the valve 200 into the native annulus. Said another way, increasing an amount of tension along the tethers 276A and 276B can be operable to transition at least the subannular member 230 (or portion thereof) between a first configuration and a second configuration. As such, the tethers 276A and 276B can be actuated (or placed in tension) and/or released in a manner similar to that described above with reference to the tethers 275.

FIG. 19 is a bottom perspective view of the valve 200 and the control device 270 and shows the subannular member 230 (and/or the proximal subannular anchoring element 234 thereof) in an at least partially extended or unactuated configuration. FIG. 20 is a bottom perspective view of the valve 200 and the control device 270 and shows the subannular member 230 (and/or the proximal subannular anchoring element 234 thereof) partially actuated such that, for example, the proximal anchoring element 234 of the subannular member 230 is drawn toward the flow control component 250. More particularly, the first tether 276A can be actuated by pulling the ends of the tether 276A proximally, which in turn, can place the tether 276A in tension and pull the attachment points through which the tether 276A is routed closer together, as shown in FIG. 20. Similarly, the second tether 276B can be actuated by pulling the ends of the tether 276B proximally, which in turn, can place the tether 276B in tension and pull the attachment points through which the tether 276B is routed closer together. In this embodiment, for example, the first tether 276A can be operable to pull the proximal anchoring element 234 inward toward the flow control component 250, while the second tether 276B can be operable to pull the sides or lateral regions of the subannular member 230 inward toward the longitudinal centerline of the valve 200. Furthermore, each of the tethers 276A and 276B can pull the respective portions of the subannular member 230 toward the supra-annular member 220 based at least in part on the connection member 278 of the control device 270 being removably attached to the supra-annular member 230 (see e.g., FIG. 18).

During deployment, an operator can actuate a proximal end portion of the control device 270 (e.g., disposed outside of the body) to, for example, pull the tether(s) 276A and/or 276B in a proximal direction, thereby folding or compressing the proximal anchoring element 234 toward the flow control component 250 and/or otherwise reconfiguring the subannular member 230 from a first configuration to a second configuration. The actuation of the control device 270 can also fold, compress, and/or draw a proximal portion of a posterior and anterior wall of the transannular member 212 inward toward the flow control component 250 (see e.g., FIG. 20). After deploying the valve 200 in the annulus of the native valve, the control device 270 can be removed or decoupled from the valve 200, the guidewire catheter 284 (and the guidewire extending therethrough) can be retracted through the waypoint or opening in the supra-annular member 220, and the delivery system can be decoupled from the valve 200 and withdrawn from the patient, leaving the deployed prosthetic valve 200 in place in the annulus of the native heart valve.

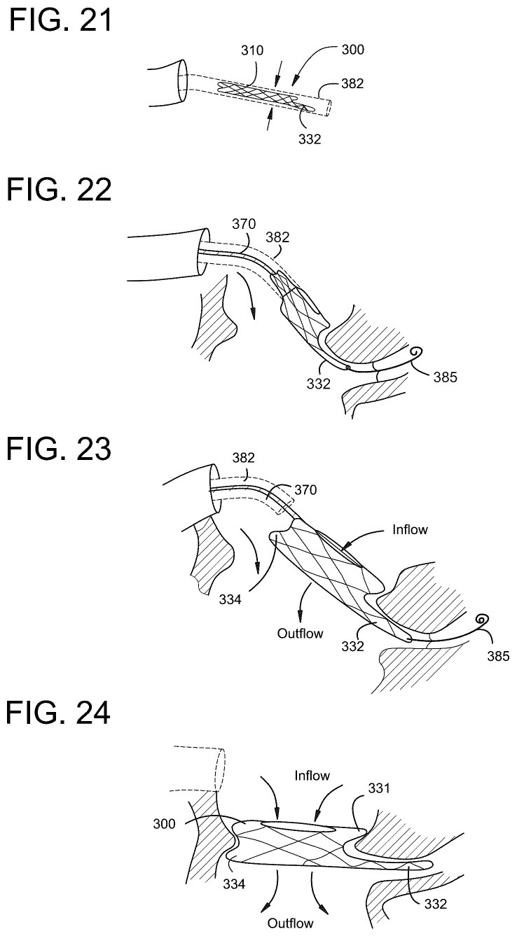

As described above, any of the valves herein can be configured for side-delivery into an annulus of a native heart valve. For example, FIGS. 21-24 illustrate a process of delivering and deploying a side-deliverable prosthetic valve. FIG. 21 is an illustration of a side perspective view of a valve 300 that is at least compressed in a vertical direction (e.g., along a central axis and/or in a direction of blood flow through the valve 300). For example, the valve 300 can include a frame 310 that has horizontally arranged diamond-shaped cells rather than traditional vertically arranged diamond-shaped cells, which can facilitate vertical compression (e.g., from top to bottom). In some implementations, the valve 300 can also be folded or compressed in a lateral direction. In the compressed configuration, the valve 300 can be loaded into a delivery catheter 382 in a side-deliverable or orthogonally deliverable position or orientation. In some implementations, side or orthogonal delivery can allow for the delivery of a valve having a larger diameter than can be delivered using traditional radial compression (e.g., radially toward a central axis). Additionally, the orthogonal delivery provides access to, for example, the tricuspid annulus from the IVC allowing the side or orthogonally delivered valve 300 to be directly expelled into (e.g., a distal subannular tab or anchoring element 332 can be aligned with and/or inserted into the tricuspid annulus.

FIG. 22 is an illustration of a side perspective view of the valve 300 being partially expelled or released from the delivery catheter 382 that allows the valve 300 or at least a portion thereof to transition from the compressed configuration to an expanded configuration. For example, in some implementations, a control device, a rigid pull/push rod, a multi-lumen catheter, and/or the like (referred to herein as “control device 370”) can be used to advance the valve 300 through the delivery catheter 382 and further used to expel the valve 300 from the delivery catheter 382 toward the native annulus. A guidewire 385 is shown extending from the delivery catheter 382, through the annulus of the native tricuspid valve and into, for example, the RVOT of the right ventricle. In some implementations, the guidewire 385 can extend to and/or through the pulmonary valve within the RVOT.

FIG. 22 further shows that as the valve 300 is released from the delivery catheter 382, the distal subannular tab or anchoring element 332 is advanced along the guidewire 385, through the annulus of the native tricuspid valve, and at least partially into the RVOT. The distal subannular tab or anchoring element 332 is configured to provide anchoring for the valve 300 while it is positioned and/or while blood flow through the valve 300 is being assessed during deployment.

FIG. 23 is an illustration of a side perspective view of the valve 300 being fully expelled or released from the delivery catheter 382 into an expanded configuration. The valve 300 is at least partially located and/or secured relative to the annulus using the distal subannular tab or anchoring element 332 against a distal subannular surface of the annulus. In some instances, the valve 300 can be temporarily held, using the control device 370, at an elevated angle above the native annulus prior to complete deployment of the valve 300. This allows a transition of blood flow from the native flow through the native valve to a flow through the prosthetic valve. For example, as the valve 300 is being deployed, native blood flow through the native tricuspid valve is transitioned to include at least partial flow around the prosthetic valve 300 and into the native annulus, and then to include at least partial flow through an inflow end and out of an outflow end of the prosthetic valve 300 into the native annulus (indicated in FIGS. 23 and 24 by the arrows labeled “Inflow” and “Outflow,” respectively). FIG. 24 further shows a proximal subannular anchoring element, tab, lower tension arm, etc. (“proximal anchoring element 334”), which can facilitate the mounting or anchoring of the valve 300 once entirely deployed in the native annulus. In some implementations, the proximal anchoring element 334 can be movable and/or otherwise reconfigurable and can be in a first configuration prior to a proximal side or end of the valve 300 being inserted into the annulus of the native valve.

FIG. 24 is an illustration of a side perspective view of the valve 300 being fully expelled or released from the delivery catheter 382 and seated into the annulus of the native tricuspid valve. In some implementations, once the valve 300 is inserted into the annulus, the proximal anchoring element 334 can be allowed to transition to a second or expanded configuration that can, for example, increase a diameter and/or perimeter of a subannular region or portion of the valve 300. In some implementations, the valve 300 can be anchored using at least the distal subannular tab or anchoring element 332 and the proximal subannular tab or anchoring element 334. In some embodiments, the valve 300 can also include a distal supra-annular (atrial) anchoring element that can also facilitate the anchoring of the valve 300 in the annulus. The delivery of the valve 300 just described allows a smooth transition from native blood flow to a full and/or complete blood flow through an inflow end of the prosthetic valve 300 and out of an outflow end of the prosthetic valve and thus, through the native annulus.

The valves 100, 200, and/or 300 are generally described above as being delivered into a heart, deployed from a delivery catheter, and seated into an annulus of a native heart to function as a prosthetic heart valve. As described above, after seating the prosthetic valves 100, 200, and/or 300, a delivery system used to deliver the valve is removed from the patient, while the prosthetic valves 100, 200, and/or 300 are left in place. In some implementations, however, it may be desirable to retrieve and/or remove a side-deliverable prosthetic valve during delivery, deployment, and/or after seating the prosthetic valve in the annulus. In some such implementations, a retrieval system and/or a retrieval portion of a delivery/retrieval system (sharing one or more common elements) can be used to engage the prosthetic valve and to retrieve the valve into the delivery catheter, a retrieval sheath, and/or the like.

For example, FIGS. 25-42 illustrate retrieval portions and/or aspects of a delivery/retrieval system 480 (referred to herein as “retrieval system 480”) configured to retrieve a prosthetic valve during delivery, deployment, and/or after seating the prosthetic valve in the annulus, according to an embodiment. In some instances, a retrieval process of a valve such as the valve 200 shown in FIGS. 10-20 can begin with removing one or more components included in a delivery portion of the delivery/retrieval system 480. For example, FIGS. 25-28 illustrate components included in the delivery portion of the delivery/retrieval system 480 used to deliver and/or deploy the valve 400 into the annulus of a native heart valve. As described above with reference to the delivery/retrieval system 180, the delivery/retrieval system 480 can include a delivery catheter (not shown) configured to provide access to a chamber of the heart (e.g., via the IVC or SVC approach).

FIG. 25 shows, for example, a delivery and/or control handle 488 that can facilitate loading of the valve 400 and/or manipulation of the valve 400 during delivery or deployment. In some embodiments, the delivery and/or control handle 488 can be similar to any of the handles of the delivery/retrieval system(s) described in the '032 PCT incorporated by reference above and thus, portions and/or aspects of the delivery and/or control handle 488 may not be described in further detail herein. In some embodiments, the delivery and/or control handle 488 can be operably coupled to and/or can be a part of a control device 470. For example, a proximal end portion of a control catheter 471 of the control device 470 can be coupled to and/or disposed in the delivery and/or control handle 488. Moreover, a proximal control end of the control device 470 can be coupled to and/or can extend from the delivery and/or control handle 488 and can allow insertion of one or more components into one or more lumen of a control catheter 471 of the control device 470, as described in the '032 PCT incorporated by reference above.

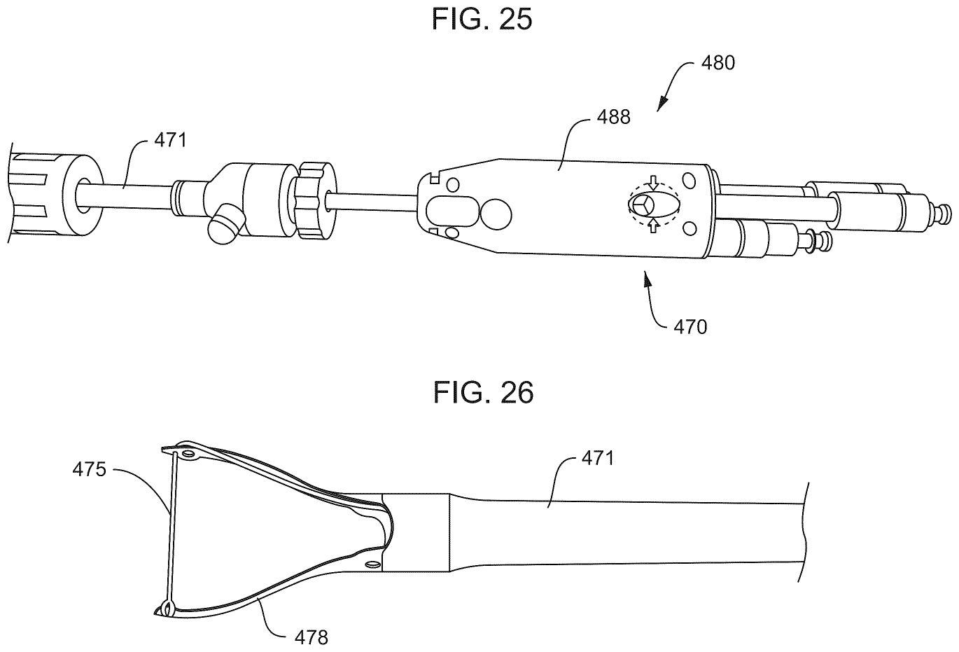

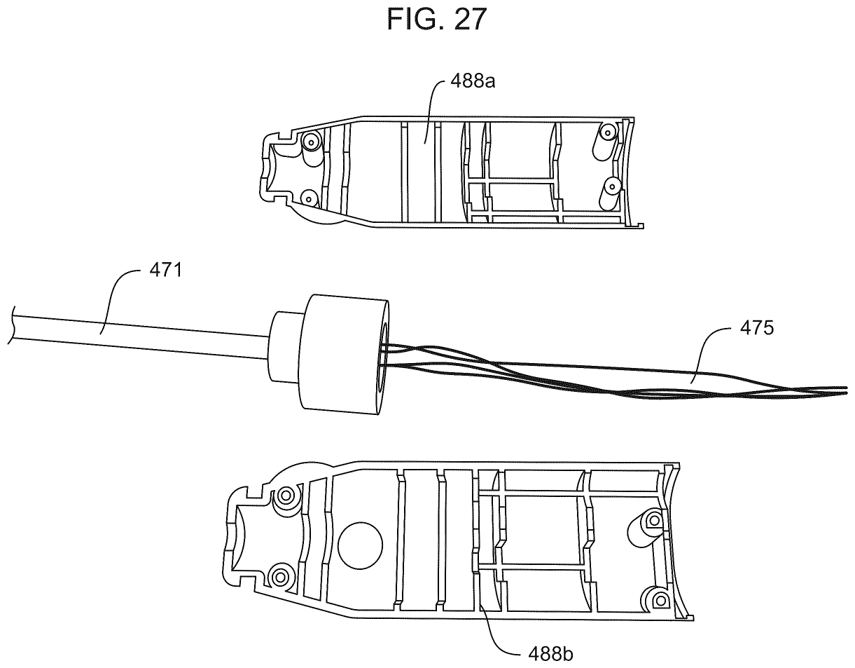

FIG. 26 is a top perspective view of a distal end portion of the control device 470 showing the control catheter 471 coupled to and/or including a connection member 478. One or more tethers 475 can be used to removably couple the connection member 478 to, for example, a supra-annular member of the valve. More particularly, the tethers 475 can be routed from the delivery and/or control handle 488, through the control catheter 471 and the connection member 478, looped around an attachment member of the valve, and routed back through the connection member 478, the control catheter 471, and delivery and/or control handle 488. The looped arrangement of the tethers 475 removably couples the connection member 478 to the supra-annular member 420 of the valve and is such that the ends of each tether 475 are disposed at and/or coupled to the delivery and/or control handle 488 to allow a user to manipulate the connection member 478 and/or the coupling between the connection member 478 and the valve, as described above with reference to the delivery/retrieval system 180 shown in FIGS. 1-9.

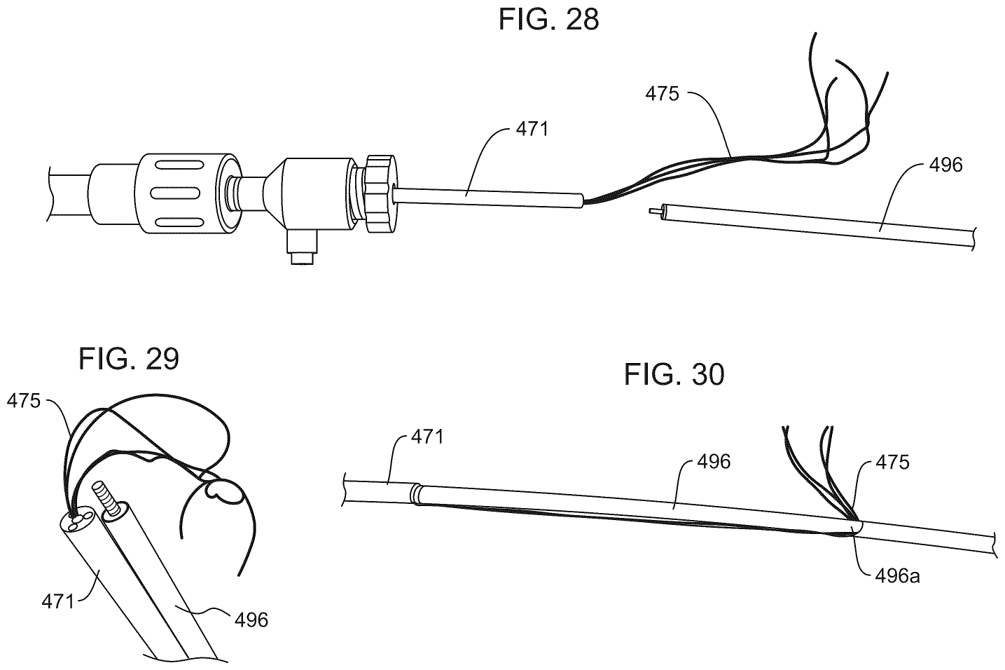

FIG. 27 shows the delivery and/or control handle 488 decoupled from the control device 470 and/or the like. For example, the delivery and/or control handle 488 can have a split body design allowing the delivery and/or control handle 488 to be separated into at least a first piece 488A and a second piece 488B. FIG. 28 shows that the tethers 475 can remain routed through the control catheter 471 when the delivery and/or control handle 488 is removed. Although not shown in FIGS. 25-28, one or more tethers (other than the tethers 475), tension members, actuators, etc. coupled to one or more portions of the valve 400 can also be removed. For example, tethers used to actuate a proximal anchoring element can be decoupled from the valve and removed from one or more lumen of the control catheter 471. Similarly, a guidewire and guidewire catheter along which the valve is advanced during the delivery can be retracted and/or removed. In some implementations, the delivery catheter also can be removed from the patient without removing the control catheter 471. For example, in implementations in which a relatively large valve is being retrieved, it may be desirable to remove the delivery catheter to allow a larger diameter retrieval sheath to be advanced along the control catheter 471 to the chamber of the heart. In other implementations, the delivery catheter can remain in place and retrieval components of the delivery/retrieval system 480 can retrieve the valve into the lumen of the delivery catheter (e.g., when retrieving relatively small valves).

While portions of the delivery/retrieval system 480 are removed, FIGS. 28-30 show that the control catheter 471 (with the connection member 478 disposed at the distal end thereof) and the tethers 475 remain attached or coupled to the valve. For example, as with delivery and deployment, the control catheter 471 can still extend through the vasculature of the patient with the connection member 478 at the distal end thereof disposed in the chamber of the heart and still coupled to the valve. With the desired delivery portions of the delivery/retrieval system 480 removed, one or more retrieval portions of the delivery/retrieval system 480 can be employed to retrieve engage and retrieve the valve 400.

FIGS. 28-30 show an exchange catheter 496 configured to be coupled to the control catheter 471 to allow the retrieval portion of the delivery/retrieval system 480 to be disposed over the control catheter 471. FIG. 29 shows a distal end proximal of the exchange catheter 496 including, for example, a threaded male coupler that can be inserted into a lumen of the control catheter 471 to form a threaded coupling therebetween. As such, the distal end portion of the exchange catheter 496 can couple to the proximal end portion of the control catheter 471, which can, for example, extend a length of the control catheter 471 (e.g., proximally). In some implementations, the additional proximal length can allow and/or allow one or more portions of the delivery and/or retrieval system 480 to be advanced over the exchange catheter 496 and at least a portion of the control catheter 471, as described in further detail herein.

With the delivery and/or control handle 488 removed, the ends of each tether 475 are unattached or not anchored and extend beyond the proximal end of the control catheter 471. FIG. 30 shows the control catheter 471 coupled to the exchange catheter 496, which includes and/or defines a feature configured to engage and/or couple to the tethers 475 to secure the end portions thereof. For example, the exchange catheter 496 can include and/or can define a skive 496A that can selectively receive the end portions of the tethers 475, thereby securing the end portions via a clamping force or the like. In other embodiments, the exchange catheter 496 can include any other suitable coupler, retainer, and/or engagement feature. In this manner, control of the distal end portion of the control device 470 (e.g., the connection member 478 and the valve 400 attached thereto) can be maintained during the retrieval process.

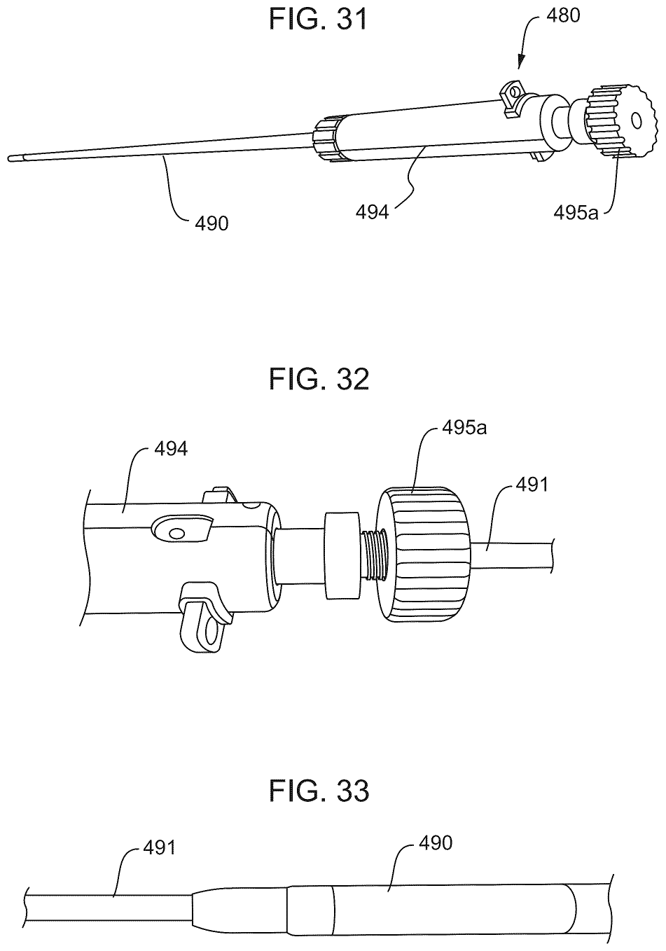

FIGS. 31-33 show a retrieval sheath 490, a retrieval element 491, and a retrieval handle 494 included in the retrieval portion of the delivery/retrieval system 480. FIG. 31 shows a proximal end portion of the retrieval sheath 490 coupled to the retrieval handle 494. The retrieval sheath 490 sheath can be, for example, a flexible catheter having any suitable size and/or diameter. In some implementations, for example, the retrieval sheath 490 and the retrieval handle 494 can replace and/or function similarly to a delivery sheath and/or the delivery and/or control handle 488 (now removed). In some such embodiments, the retrieval sheath 490 and the retrieval handle 494 can be similar to and/or substantially the same as the delivery sheath and/or handles described in the '032 PCT incorporated by reference above. In some implementations, the retrieval sheath 490 can be a steerable or at least partially steerable catheter having at least a larger inner diameter than the delivery sheath it replaces. In other implementations, the delivery sheath can remain in place and the retrieval handle 494 can be coupled to a proximal end thereof. In some instances, it may be desirable to use the retrieval sheath 490 that is larger than the delivery sheath used to deliver the valve to facilitate and/or accommodate compression of the valve 400 without a loading device and/or the like. For example, in some instances, the retrieval sheath 490 can be a 38 Fr catheter, while a delivery sheath can be a 28 Fr catheter. In some instances, configuring the retrieval sheath 490 with a larger diameter can allow, for example, retrieval of relatively large valves. In other instances, the retrieval handle 494 can be coupled to an existing (placed) delivery sheath when, for example, retrieving relatively small valves.

The retrieval handle 494 coupled to the proximal end of the retrieval sheath 490 can have any suitable shape, size, and/or configuration and can provide, for example, at least a portion of a user interface for the retrieval sheath 490. For example, the retrieval handle 494 can have a size, shape, and/or configuration similar to the delivery and/or control handle 488 that was previously removed. FIG. 32 shows the proximal end portion of the retrieval handle 494 including a coupler 495A configured to allow one or more devices to couple to and/or otherwise engage the retrieval handle 494. For example, in some embodiments, the coupler 495A can be a collet or the like that can be used to secure one or more devices of the delivery/retrieval system 480, as described in further detail herein. The distal end portion of the retrieval handle 494 is coupled to the retrieval sheath 490. In some embodiments, the distal end portion of the retrieval handle 494 can include one or more control features and/or the like allowing a user to steer or at least partially steer a distal end portion of the retrieval sheath 490 (e.g., in at least one direction or in at least one plane).

FIGS. 32 and 33 show the retrieval element 491 extending through the proximal end of the retrieval handle 494 and a distal end of the retrieval sheath, respectively. As shown, the retrieval element 491 can be and/or can include a catheter that can be movable through the retrieval sheath 490 and the retrieval handle 494. In some embodiments, a lumen defined by the catheter of the retrieval element 491 can receive at least a portion of the control catheter 471 allowing the retrieval element 491 to be moved selectively over the control catheter 471. In some implementations, a dilator can be disposed a distal end of the retrieval sheath 490 to facilitate advancement of the retrieval sheath 490 through the body to the chamber of the heart.

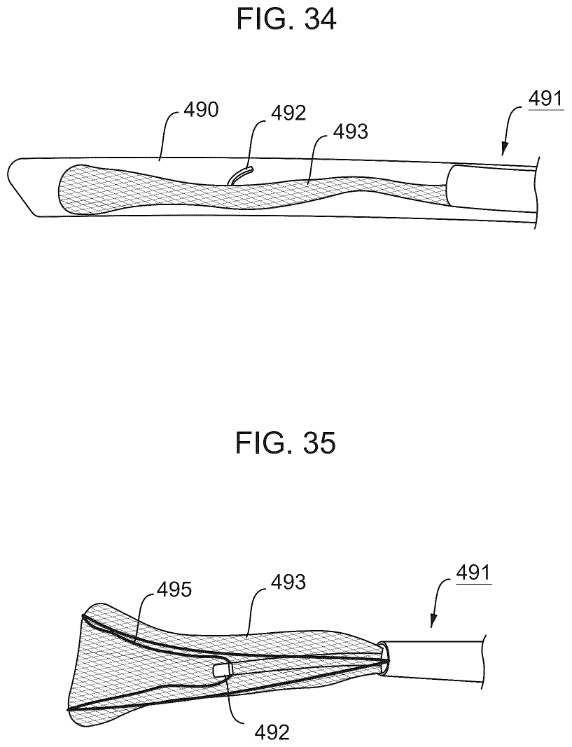

FIGS. 34-39 are various views showing a distal end portion of the delivery/retrieval system 480, showing the retrieval element 491, an engagement member 492, a guide member 493, and an optional actuator 495. The retrieval element 491 can be any suitable device, element, member, and/or the like having any suitable size. As described above, the retrieval element 491 can be and/or can include a catheter defining a lumen with a sufficiently large diameter to allow the retrieval element 491 to be disposed over and advanced along the control catheter 471 (not shown in FIGS. 34-39). The engagement member 492 and the guide member 493 are both coupled to and/or extend from the distal end portion of the retrieval element 491 (e.g., catheter). As described in further detail herein, the optional actuator 495 can extend through the retrieval element 491 (or the retrieval sheath 490) and can be at least temporarily coupled to the engagement member 492 and/or the guide member 493 allowing a user to actuate, manipulate, and/or reconfigure one or more portions thereof.

FIG. 34 is a side view showing the retrieval element 491, the engagement member 492, and the guide member 493 disposed, for example, in the retrieval sheath 490 with at least the guide member 493 in a compressed or at least partially compressed state (e.g., a delivery state) for advancement into a chamber of the heart. The engagement member 492 coupled to the distal end of the retrieval element 491 can be any suitable device, member, feature, etc. that is configured to engage one or more portions of the prosthetic valve to facilitate retrieval thereof, as described in further detail herein. For example, in this embodiment, the engagement member 492 is shown arranged and/or configured as a hook that extends from the distal end of the retrieval element 491. In some embodiments, the engagement member 492 is formed from, for example, a shape-memory material such as Nitinol or the like. Accordingly, in some implementations, the engagement member 492 can be configured to transition between two or more configurations such as at least a delivery configuration and a deployment/engagement configuration. In such implementations, the engagement member 492 can have a relatively low profile when in the delivery configuration and can be allowed to transition (e.g., when extending distal to the retrieval sheath 490 and/or control catheter 471) to the deployment/engagement configuration in which the engagement member 492 forms and/or assumes the hook or other desirable shape.

In other implementations, the engagement member 492 can form the hook or other desirable shape and need not be configured to transition. For example, as shown in FIG. 34, during delivery of the retrieval portion of the delivery/retrieval system 480, the guide member 493 and the engagement member 492 can be disposed in the retrieval sheath 490 in a distal position relative to the retrieval element 491. Although not shown, the control catheter 471 may also extend through the retrieval sheath 490. The inner diameter of the retrieval sheath 490, however, may be sufficient to allow the engagement member 492 to be advanced therethrough without being transitioned to a collapsed, compressed, and/or flattened configuration.

The guide member 493 can be any suitable device, member, feature, etc. For example, the guide member 493 can be a scoop, tongue, flange, and/or any other suitable feature extending from the retrieval element 491 in a curved or arcuate path. In some embodiments, the guide member 493 is formed out of one or more braided, mesh, and/or tube shape-memory materials such as Nitinol. Accordingly, the guide member 493 can be configured to transition between two or more configurations such as at least a delivery configuration (FIG. 34) and a deployment/guiding configuration (FIGS. 35 and 36). In such implementations, the guide member 493 can be relatively compact and/or compressed when in the delivery configuration allowing the guide member 493 to be advanced, for example, through the retrieval sheath 490, as shown in FIG. 34. The guide member 493 can be allowed to expand when released from the retrieval sheath 490 (e.g., when distal thereto) to the deployment/guiding configuration. In the deployment/guiding configuration, the braided/mesh material of the guide member 493 can expand to form the scoop or scoop-like shape (see e.g., FIGS. 35 and 36).

In some embodiments, the guide member 493 may have any number of additional configurations, due at least in part to being formed from a relatively flexible braided/mesh material. For example, in some implementations, when disposed in a chamber of the heart (e.g., at least partially expanded/extended), one or more portions of the guide member 493 can be actuated, manipulated, reconfigured, placed in tension, bent, rolled (or at least partially rolled), folded (or at least partially folded), torqued, etc. (referred to herein for simplicity as “actuated”) allowing a user to move or control a placement of one or more portions of the guide member 493 (and/or engagement member 492) relative to, for example, the anatomy of the heart and/or the prosthetic valve at least partially deployed in the heart.

For example, the delivery/retrieval system 480 can include any suitable device(s), mechanism(s), feature(s), etc. (e.g., such as the actuator 495 shown in FIGS. 35-39), allowing manipulation of one or more portions of the guide member 493 and/or engagement member 492. In some embodiments, the guide member 493 can be actuated into one or more configurations having and/or forming a desired radius of curvature that may be controlled to accommodate the anatomy of chamber(s) of the heart. For example, the guide member 493 may be actuated into a tighter curvature to access the prosthetic valve from a sharper or tighter angle and/or to guide the prosthetic valve along the sharper or tighter angle into the retrieval sheath 490. In some embodiments, the shape of the guide member 493 may be adjusted during retrieval to reduce a likelihood of catching or snagging one or more portions of the prosthetic valve on an inner surface of the heart. Moreover, in some implementations, actuating the guide member 493 can allow at least a portion of the guide member 493 and/or the engagement member 492 to be advanced relative to and beyond a portion of the prosthetic valve (e.g., advanced in a distal direction beyond the proximal anchoring element) without getting caught or snagged prior to being placed in a desirable relative position.

FIGS. 36-39 illustrate an example implementation of the optional actuator 495, which may be used to actuate the guide member 493. In this embodiment, for example, the actuator 495 is configured to exert a force on the guide member 493 to adjust a shape of the guide member 493. In some embodiments, the actuator 495 may include tethers (e.g., sutures, cables, tensile members, rods, wires, hypotubes, connectors, and/or the like) coupled to the guide member 493 and/or the engagement member 492 and configured to exert the force on the guide member 493. In some embodiments, the actuator 495 may be and/or may include a tether that extends through the retrieval sheath 490 and/or the retrieval element 491 such that a portion of the tether may couple to the guide member 493 and/or the engagement member 492 at the distal end portion of the delivery/retrieval system 480.

For example, in some implementations, a first end and a second end of the tether (actuator 495) may extend through the retrieval sheath 490 and/or the retrieval element 491 to a proximal end portion of the delivery/retrieval system 480, and the middle portion of the tether (actuator 495) may be coupled to the guide member 493 and/or the engagement member 492. Similarly stated, the actuator 495 can be and/or can include a tether or suture that can be “doubled back” or otherwise routed through the retrieval sheath 490 and/or retrieval element 491 such that each of a proximal end and a distal end of the tether or suture are disposed outside the retrieval system and body of the patient. In some embodiments, the middle portion of the tether may be configured to extend along an underside of the guide member 493 (e.g., along the side of the guide member 493 configured to face away from the prosthetic valve), over and/or through a distal end portion of the guide member 493, and looped, curved, and/or otherwise disposed about a portion of the engagement member 492 (e.g., the hook). Therefore, when the distal end of the retrieval element 491 is disposed in the chamber of the heart, a force may be applied to the both ends of the tether at the proximal end portion of the delivery/retrieval system 480 (e.g., outside the body) such that the middle portion of the tether (now in a distal position and at least temporarily coupled to the engagement member 492) exerts a force on the distal end portion of the guide member 493 to bend the distal end portion away from a centerline of the delivery/retrieval system 480 (e.g., away from the prosthetic valve), thereby tightening the curvature of the scoop or scoop-like shape of the guide member 493. In some embodiments, after the engagement member 492 has engaged the prosthetic valve (e.g., a proximal anchoring element of the prosthetic valve), a force exerted on the guide member 493 and/or engagement member 492 via the actuator 495 may be reduced or released such that the guide member 493 may transition back to an initial, biased, relaxed, and/or unactuated configuration (e.g., the deployment configuration (FIG. 36)). The retrieval element 491, the engagement member 492, the guide member 493, and the prosthetic valve may then be pulled into the retrieval sheath 490.

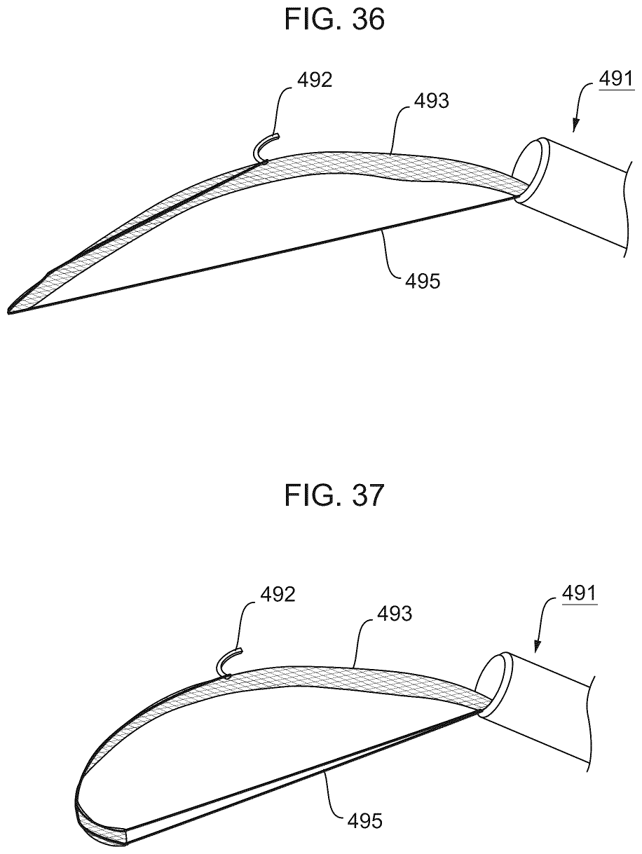

In some embodiments, a force can be exerted on the first end and/or the second end portions of the tether in a proximal direction to apply tension along the tether (actuator 495). With a portion of the tether routed, for example, through and/or around a distal end portion of the guide member and with the middle portion of the tether (e.g., the portion in or proximate to a distal position) wrapped or disposed about the engagement member 493, the force pulls and/or bends at least the distal end portion of the guide member 493, as shown in FIG. 37. In some embodiments, the force applied to the first and/or second end portions of the tether (actuator 495) may control a degree to which the guide member 493 curves (e.g., control a radius of curvature of the guide member 493). For example, a greater force applied to the first and/or second end portion of the tether (actuator 495) in the proximal direction may decrease the radius of curvature of the guide member 493.

While the actuator 495 and/or a tether of the actuator 495 is described as being in a “doubled back” configuration, in some embodiments, a first end portion of a tether may extend through the retrieval sheath 490 and proximal to the delivery/retrieval system 480 (e.g., outside the body of the patient), and a second end portion of the tether may be configured to be at least temporarily fastened to one or more attachment points along the distal end portion of the delivery/retrieval system (e.g., the retrieval element 491, an inner surface of the retrieval sheath 490, a portion of the engagement member 492, a distal end portion of the guide member 493, a proximal end portion of the guide member 493, etc.). In some embodiments, the second end portion of the tether may be configured to decouple or detach from such an attachment point after the prosthetic valve has been at least partially retrieved such that the tether may be removed and/or retracted from the retrieval sheath 490 (the catheter). For example, the tether may be configured such that a force exerted on the tether in a particular direction and/or with a particular magnitude may decouple the second end of the tether from the attachment point (e.g., a break-away attachment or the like). In some embodiments, the second end of the tether may be at least temporarily fastened so that the tether may be removed more quickly than if both the first end and the second end of the tether were to extend through the proximal end of the delivery/retrieval system 480. In some implementations, such an arrangement may be used when the prosthetic valve is being partially retrieved, reseated, repositioned, etc.

In some embodiments, the tether may be secured relative to the guide member 493 to prevent slipping across the guide member 493 in a lateral direction. For example, one or more portions of the tether may be disposed through one or more openings in the guide member 493 (e.g., one or more cells or one or more portions of a mesh of the guide member 493) to prevent slippage along the lateral direction while still allowing movement in the proximal/distal direction. In some embodiments the tether may be secured (tied, sewn, sutured, etc.) to the guide member 493 with sutures to prevent slippage in the lateral direction while allowing movement of the tether in the proximal/distal direction.

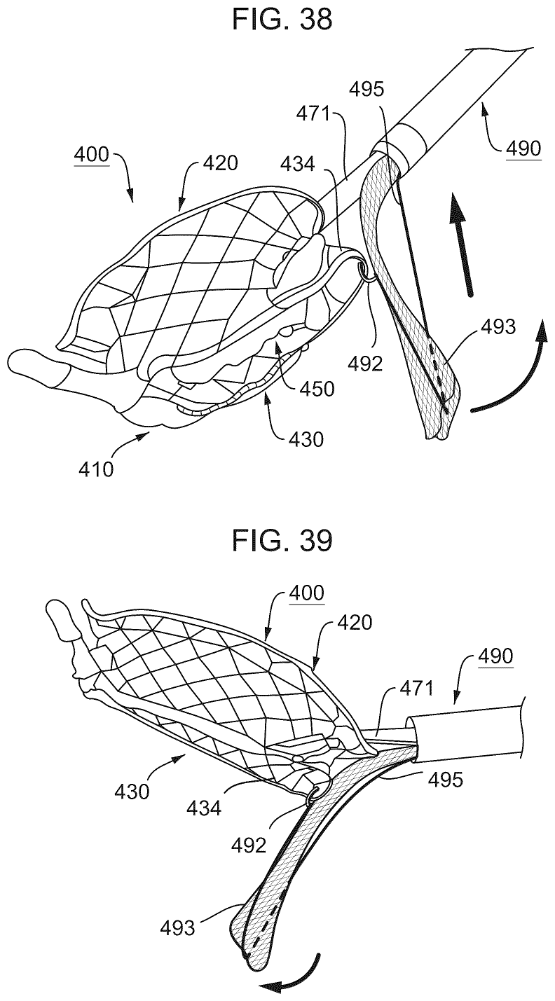

FIGS. 38-41 show the retrieval element 491, the engagement member 492, the guide member 493, and the actuator 495 being used to engage a valve 400 for a retrieval process. The valve 400 can be, for example, similar to or substantially the same as the valve 200 described in detail above with reference to FIGS. 10-20. Accordingly, the valve 400 can have and/or can form an outer support frame 410 with a flow control component 450 mounted therein. FIGS. 38 and 39 show the outer support frame 410 having a supra-annular member 420 and a subannular member 430. As described above with reference to the valve 200, the supra-annular member 420 of the valve 400 can be releasably coupled to the distal end portion of the control catheter 471 (e.g., via the connection member 478 not shown in FIGS. 38-41). The subannular member 430 is shown forming a proximal subannular anchoring element 434 similar to or substantially the same as the proximal subannular anchoring element 234 of the valve 200.

FIGS. 38 and 39 are side views showing the engagement member 492 and the guide member 493 extending distal to the retrieval sheath 490 into an operable position and/or arrangement relative to the valve 400. For example, the engagement member 492 is shown engaging and/or hooking one or more portions of the subannular member 430 of the valve 400 such as the proximal subannular anchoring element 434. More specifically, the proximal subannular anchoring element 434 can have and/or can form a rim (e.g., a wire rim covered in biocompatible material, cloth, etc.), which can be engaged and/or hooked by the engagement member 492.

The guide member 493 is shown extending in the retrieval, guiding, and/or expanded configuration from a proximal end at or near the retrieval sheath 490 to a distal end below or substantially below the valve 400. As such, the guide member 493 can be configured to guide one or more portions of the valve 400 into the retrieval sheath 490. For example, during retrieval, the valve 400 can be pulled in a proximal direction toward the distal end of the retrieval sheath 490. With the valve 400 in the retrieval, guiding, and/or expanded configuration (FIG. 38), drawing the valve 400 toward and/or into the retrieval sheath 490 can begin to transition the valve 400 from the expanded configuration to the compressed configuration, as shown in FIG. 39. As the valve 400 is compressed, one or more portions, edges, etc. of the valve 400 may otherwise become snagged on the distal end of the retrieval sheath 490, thereby inhibiting retrieval. Accordingly, the guide member 493 in the expanded configuration can guide the one or more portions, edges, etc. of the valve 400 into the retrieval sheath 490 to limit and/or substantially prevent snagging.

As described above, in some embodiments, the delivery/retrieval system 480 may include the optional actuator 495 that can be used to facilitate placement of the guide member 493 and the engagement member 492 relative to the prosthetic valve 400 (e.g., at least the proximal anchoring element 434 thereof). For example, a user may exert a proximally directed force at the proximal end portion of the actuator 495, which in turn may increase a tension along the actuator 495. As shown by the arrows in FIG. 38, the proximally directed force along the actuator 495 is operable to pull the distal end portion of the guide member 493 in the direction of and/or closer to the distal end of the retrieval sheath 490. As such, at least the distal end portion of the guide member 493 is bent, flexed, pivoted, rolled (or at least partially rolled), and/or rotated in a direction away from the prosthetic valve 400. In some implementations, actuating the guide member 493 (e.g., via the actuator 495) can allow at least a portion of the guide member 493 and the engagement member 492 to be placed in a distal position relative to at least the proximal anchoring element 434 of the valve 400. Once in the desired position, the tension along the actuator 495 can be released, allowing the guide member to bend, flex, pivot, roll, and/or rotate in a direction toward the prosthetic valve 400, as indicated by the arrow in FIG. 39. Similarly stated, the force exerted on the actuator 495 can be removed, thereby releasing the tension along the actuator 495, which in turn, can allow the guide member 493 to move or transition to a biased, initial, and/or otherwise an unactuated configuration (e.g., the retrieval configuration). Accordingly, the retrieval element 491 can be pulled in a proximal direction, which in turn, moves the guide member 493 and the engagement member 492 in a proximal direction relative to the prosthetic valve 400 until, for example, the engagement member 492 engages and/or hooks the proximal anchoring element 434. The prosthetic valve 400 can then be pulled and/or retrieved into the retrieval sheath 490, as described above.

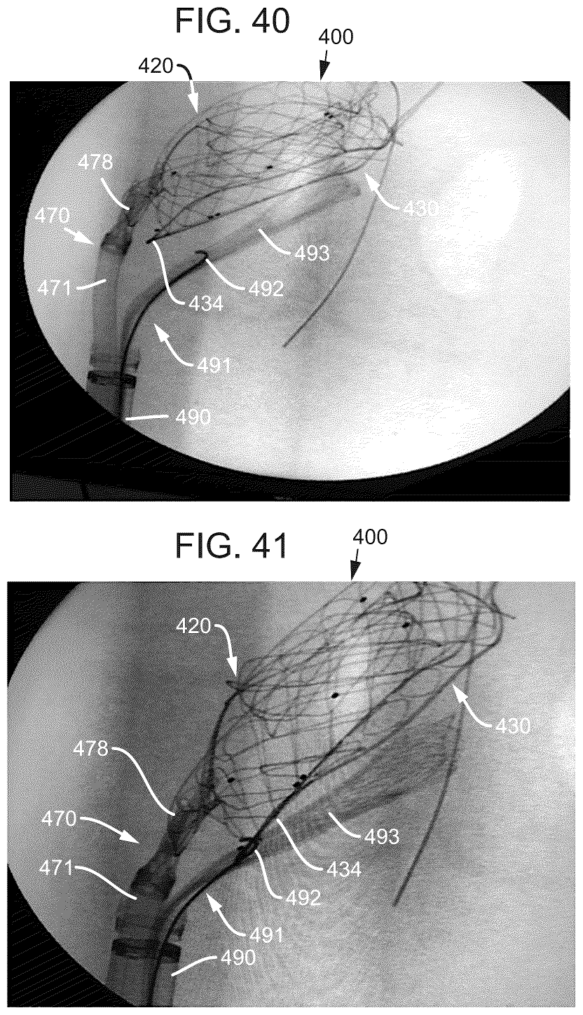

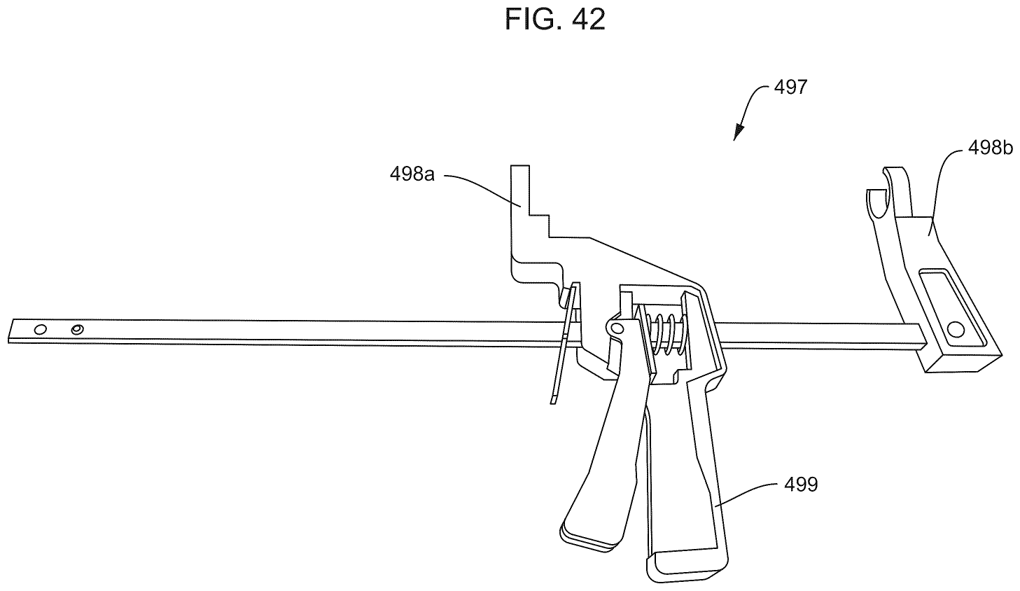

FIGS. 40 and 41 are side view fluoroscopic black and white photographs showing the valve 400 in a chamber of the heart. The control catheter 471 is shown extending from the retrieval sheath 490 and the retrieval element 491 with the connection member 478 in contact with and coupled to the supra-annular member 420 of the valve 400. FIG. 40 shows the engagement member 492 and the guide member 493 distal to the retrieval sheath 490 but not yet engaging the valve 400, while FIG. 41 shows the engagement member 492 hooking an edge, rim, and/or wire frame structure of the proximal subannular anchoring element 434. With the connection member 478 of the control catheter 471 in contact with the supra-annular member 420 of the valve 400 and the engagement member 492 of the retrieval element 491 in contact with the proximal subannular anchoring element 434, proximal movement of the control catheter 471 and the retrieval element 491 can be operable to pull, retract, and/or retrieve the valve 400 into the retrieval sheath 490, as described in further detail below.

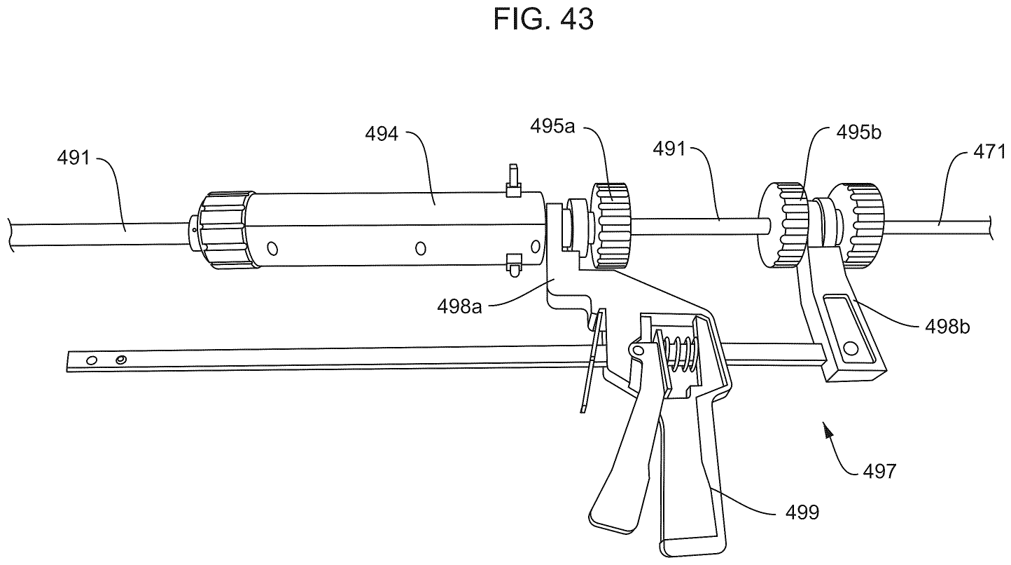

FIGS. 42 and 43 show a retractor 497 of the delivery/retrieval system 480 that can be used to move the control catheter 471 and the retrieval element 491 in a proximal direction to retrieval the valve 400 into the retrieval sheath 490. The retractor 497 can be any suitable shape, size, and/or configuration. For example, in the embodiment shown in FIGS. 42 and 43, the retractor 497 is shown as a ratchet mechanism and/or the like that can be manipulated to exert a force on one or more components to which it is coupled. In some embodiments, the ratchet mechanism (or other retractor 497) can provide a mechanical advantage that can aid in exerting a force to compress the valve 400 and retract the valve 400 into the retrieval sheath 490. As described above, in some instances, forces encountered with retrieving the valve 400 can be larger than forces encountered during delivery of the valve 400 as loading devices, etc. are not disposed in the chamber of the heart. Thus, the retractor 497 can allow a user to exert the forces associated with pulling the valve 400 into the retrieval sheath 490, which otherwise may be difficult to achieve via manual traction (e.g., pulling by hand or the like).

FIG. 42 show the retractor 497 having a first end portion 498A, a second end portion 498B, and a ratcheting handle 499. FIG. 43 shows the first end portion 498A coupled to the coupler 495A (e.g., a “first coupler”) at the proximal end portion of the retrieval handle 494. The second end portion 498B is shown coupled to a second coupler 495B, which in turn, is coupled to the proximal end portions of the control catheter 471 and the retrieval element 491. The second coupler 495B, which can be similar to or substantially the same as the first coupler 495A, is at least temporarily coupled to and/or disposed about a proximal end portion of the control catheter 471. In some implementations, the first coupler 495A of the retrieval handle 494 can be in a relatively fixed or locked position relative to the ratcheting handle 499 of the retractor 497. The second coupler 495B can be secured and/or coupled to the proximal end portions of the control catheter 471 and the retrieval element 491 and maintained in a fixed or locked position relative thereto.

The retractor 497 can be configured to transition between any number of configurations, each of which can correspond to a different distance defined between the first end portion 498A and the second end portion 498B. Accordingly, in some instances, a first distance can be defined between the end portions 498A, 498B of the retractor 497 (and thus, between the first coupler 495A and the second coupler 495B) when the retrieval element 491 is in a desired position relative to the valve 400 but prior to pulling the valve 400 into the retrieval sheath 490. When ready to pull the valve 400 into the retrieval sheath 490, a user can manipulate the ratcheting handle 499 to, for example, move the second end portion 498B of the retractor 497 relative to the first end portion 498A of the retractor 497.

With the couplers 495A, 495B coupled to the retrieval handle 494 and the control catheter 471 and retrieval element 491, respectively, the increase in the distance (e.g., to a second distance greater than the first distance) between the end portions 498A, 498B moves the distal end portions of the control catheter 471 and the retrieval element 491 relative to the retrieval sheath 490. In some implementations, the proximal movement of at least the retrieval element 491 can be such that the engagement member 492 exerts a force on the proximal subannular anchoring element 434 that can aid in transitioning the valve 400 from the expanded configuration shown in FIG. 42 to the compressed configuration.

As described above, the arrangement of the guide member 493 can be such that the guide member 493 guides and/or directs one or more portions or edges of the valve 400 into the retrieval sheath 490. For example, the guide member 493 can be configured to guide the proximal subannular anchoring element 434 and/or an edge of the inner frame of the flow control component 450 past the distal edge of the retrieval sheath 490 and into the lumen defined by the retrieval sheath 490. Moreover, the arrangement of the retractor 497 can provide a mechanical advantage operable in generating a desired amount of force associated with and/or otherwise needed to pull the valve 400 into the retrieval sheath 490 while the valve 400 is being compressed at substantially the same time. In this manner, the delivery/retrieval system 480 can be used to retrieve a valve at least partially deployed in a chamber of a heart. In some instances, once the valve 400 is retracted and/or retrieved into the retrieval sheath 490, the delivery/retrieval system 480 can be retracted and withdrawn from the body of the patient.

Although not shown in FIGS. 25-43, the valve 400 can include one or more features configured to facilitate retrieval of the valve 400 into the retrieval sheath 490. For example, as described above, the valve 400 can include a wire frame structure that is covered in biocompatible material, cloth, fabric, tissue, etc. The biocompatible material can be secured to the wire frame structure, for example, by sewing and/or stitching the material around the wire frame structure. In some embodiments, one or more sutures can be used or sewn on or in the biocompatible material between, for example, two or more cells of the wire frame structure, which can limit and/or substantially prevent edges of the cells from snagging, hanging, and/or otherwise engaging a distal end of the retrieval sheath 490.

As another example, in some embodiments, the valve 400 can include a posterior-septal (PS) tab that can help stabilize the valve 400 in the annulus of the native valve. In such embodiments, the PS tab can extend in proximal direction and can include one or more sutures configured to limit and/or constrain movement of the PS tab. In some instances, however, the PS tab can snag or engage the distal end of the retrieval sheath 490 during retrieval. Accordingly, in some embodiments, the valve can include one or more sutures configured to break, rip, tear, and/or otherwise loosen in response to a force associated with retrieving the valve 400. For example, if the PS tab snags and/or engages the distal end of the retrieval sheath 490, a force associated with the contact can be sufficient to break-away the one or more sutures limiting movement of the PS tab. Without the one or more sutures, the PS tab may be allowed to bend, flex, and/or fold back on itself, allowing the PS tab to be advanced past the distal edge of the retrieval sheath 490 and into the lumen thereof.

In some implementations, a retrieval process can include pre-compressing the valve 400 and/or the connection member 478 of the control device 470 prior to pulling the valve 400 into the retrieval sheath 490. For example, pre-compression of the valve 400 can include (i) suturing a proximal subannular anchoring element against an underside of an atrial or supra-annular collar or member and/or (ii) pinching proximal sidewall hips of the prosthetic valve. Similarly, in some implementations, the connection member 478 or yoke at a distal end of the control catheter 471 can be pre-compressed or pre-tensioned, which in turn, can reduce a lateral extent of the connection member 478 as well as partially folding the valve 400 and/or otherwise biasing the valve 400 such that that valve 400 can be folded with an application of less external force than may otherwise be used to fold the valve 400.

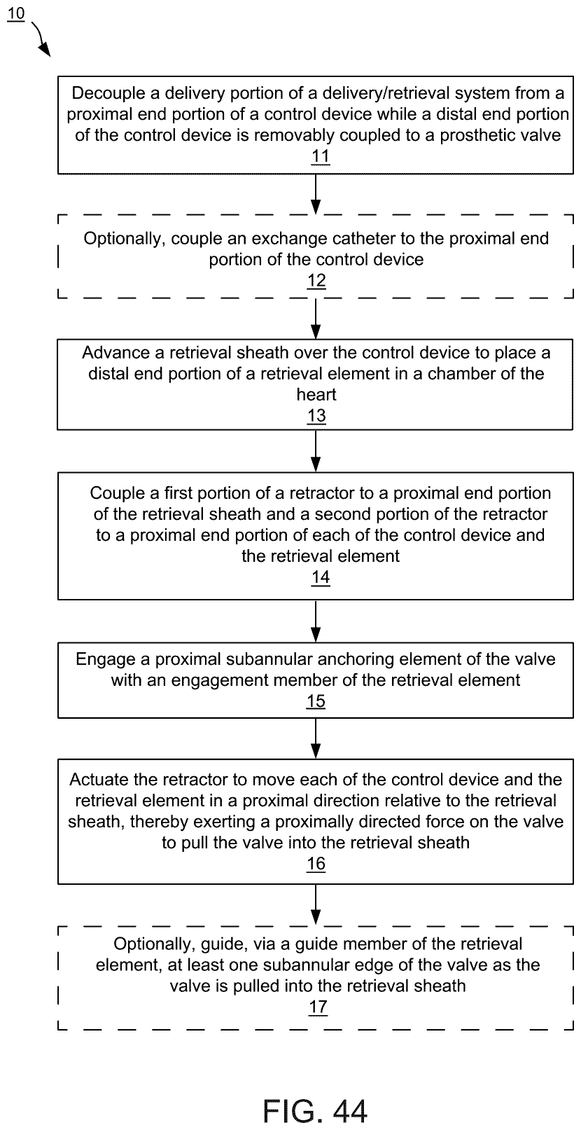

Referring now to FIG. 44, a flowchart is shown illustrating a method 10 of using a delivery/retrieval system to selectively retrieve a side-deliverable transcatheter prosthetic valve during at least one of delivery and deployment, according to an embodiment. The valve can be substantially similar to any of those described herein such as the valves 100, 200, 300, and/or 400 and/or any of those described in the '032 PCT incorporated by reference herein. For example, the valve can include an outer support frame and an (inner) flow control component that is mounted in and/or to the outer support frame. The outer support frame can include, for example, a supra-annular member or region, a subannular member or region, and a transannular member or region coupled therebetween. The flow control component is mounted to the outer support frame such that is extends through a portion of the transannular member or region, as described above. Moreover, the valve is compressible along a central axis parallel to a fluid flow direction through the valve and a lateral axis orthogonal and/or perpendicular to the central axis.

The delivery/retrieval system can be similar to or substantially the same as any of the delivery/retrieval systems described herein (e.g., the delivery/retrieval system 180 and/or 480). Accordingly, the delivery/retrieval system can include a delivery portion or delivery component(s) used to deliver the valve into a chamber of the heart, and a retrieval portion or retrieval component(s) selectively or optionally used to retrieve the valve from the chamber of the heart.

As shown in FIG. 44, the method 10 includes decoupling the delivery portion of the delivery/retrieval system from a proximal end portion of a control device disposed outside the patient while a distal end portion of the control device is disposed in the chamber of the heart and removably coupled to a supra-annular surface of the prosthetic valve, at 11. For example, in some implementations, the delivery portion can include a delivery sheath, a delivery and/or control handle, and/or any other suitable component, device, etc. In some instances, decoupling the delivery portion of the delivery/retrieval system can provide access to the proximal end portion of the control device allowing the retrieval portion of the delivery/retrieval system to be assembled and/or used.

Optionally, an exchange catheter is coupled to the proximal end portion of the control device, at 12. In some embodiments, for example, the exchange catheter can form a threaded coupling with a proximal end portion of a control catheter of the control device. The exchange catheter can be configured to extend a proximal length of the control catheter, which in turn, can allow the retrieval portion of the delivery/retrieval system to be advanced over and/or along the control catheter. Moreover, in some implementations, the control device used during delivery can be retained in place while other portions of the delivery portion are removed. As described in detail above, the distal end portion of the control device can include a connection member that is releasably secured to the supra-annular member of the valve via a set of tethers. The tethers can be routed through the control device and coupled to the valve such that both ends of each tether extends proximal to the control catheter. In such implementations, the exchange catheter can include and/or define a feature (e.g., a skive or the like) that can couple to and/or secure the end portions of the tethers allowing a user to control the distal end portion of the control device and thus, at least partially control the valve. Accordingly, in some implementations, the optional step of coupling the exchange catheter to the proximal end portion of the control device can also include securing the end portions of the tethers via the exchange catheter.

A retrieval sheath is advanced over the control catheter to place a distal end portion of a retrieval element distal to the retrieval sheath in the chamber of the heart, at 13. In some embodiments, the retrieval element can be and/or can include a retrieval catheter having a lumen that receives the control catheter of the control device. In some such embodiments, each of the retrieval element and the retrieval sheath is advanced over the control catheter. In other such embodiments, the retrieval sheath can be advanced over the control catheter and once a distal end portion of the retrieval sheath is in a desired position within the chamber of the heart, the retrieval element can be advanced over the control catheter and within the retrieval sheath. In some embodiments, a dilator is used to facilitate advancement of the retrieval sheath through the vasculature of the patient and is withdrawn from the patient prior to advancing the retrieval element. Moreover, the distal end portion of the retrieval element (e.g., a distal end portion of the retrieval catheter) can include an engagement member and a guide member, each of which is placed distal to the retrieval sheath in the chamber of the heart when the retrieval element is advanced to a desired position. In some implementations, a proximal end portion of the retrieval element can be coupled to the proximal end portion of the control device (e.g., via a coupler, collet, and/or the like).

A first portion of a retractor is coupled to a proximal end portion of the retrieval sheath and a second portion of the retractor is coupled to a proximal end portion of each of the control device and the retrieval element, at 14. More particularly, the proximal end portion of the retrieval sheath can include and/or can be coupled to a retrieval handle, as described above with reference to the retrieval sheath 490. A proximal end portion of the retrieval handle can include a coupler, collet, and/or the like to which the first end portion of the retractor is coupled. Furthermore, the second end portion of the retractor can be coupled to, for example, the coupler, collet, etc. coupling the proximal end portions of the retrieval element and the control catheter, as described above with reference to the delivery/retrieval system 480.

A proximal subannular anchoring element of the prosthetic valve is engaged with and/or by the engagement member of the retrieval element, at 15. For example, in some implementations, a distal end portion of the retrieval element can include and/or can be coupled to at least the engagement member and a guide member configured to facilitate retrieval or at least partial retrieval of the prosthetic valve. In some implementations, the delivery/retrieval system may include an optional actuator that can be manipulated to actuate the engagement member and/or the guide member to allow the engagement member and/or guide member to be moved into a desired position relative to the proximal subannular anchoring element without snagging or catching on the prosthetic valve prior to being in the desired position. Once in the desired position, the engagement member can be moved (e.g., in a proximal direction) into engagement with the proximal subannular anchoring element after the retractor is coupled to the proximal end portions of the retrieval sheath, control device, and retrieval element. As described above, the engagement member can be, for example, a hook or the like that can hook an edge, rim, etc. of the proximal subannular anchoring element.

With the engagement member engaging the proximal subannular anchoring element, the retractor can be actuated to move each of the control device and the retrieval element in a proximal direction relative to the retrieval sheath and to pull the valve into the retrieval sheath, at 16. For example, in some implementations, the retractor can include a ratcheting handle or other actuator that can be manipulated to, for example, increase a distance between the first end portion and the second end portion of the retractor. As described above, the first end portion of the retractor can be coupled to the coupler at the proximal end portion of the retrieval handle and the second end portion of the retractor can be coupled to the coupler connected to the proximal end portions of the retrieval element and the control device. In this manner, the retractor can be configured to increase a distance between the first and second end portions, which in turn, increases a distance between the couplers. The movement of the second end portion relative to the first end portion is operable to move the control device and the retrieval element in a proximal direction relative to the retrieval sheath. With the control device and the retrieval element being coupled to the supra-annular member and the subannular member of the valve, respectively, the proximal movement of the control device and the retrieval element relative to the retrieval sheath pulls the valve toward and/or into the distal end portion of the retrieval sheath. In some instances, pulling the valve into the retrieval sheath can compress the valve allowing the valve to be disposed in a lumen defined by the retrieval sheath. Said another way, the control device and the retrieval element can pull the prosthetic valve into the retrieval sheath with a force (e.g., a proximally directed force) sufficient to overcome a resistance to proximal movement resulting from the prosthetic valve contacting the distal end of the retrieval sheath. In this manner, the distal end of the retrieval sheath can act as a die or other surface that squeezes, pinches, folds, compresses, and/or otherwise transitions the prosthetic valve as the prosthetic valve is moved into the lumen of the retrieval sheath.

In some implementations, the connection member of the control device can be compressed prior to moving the prosthetic valve into the retrieval sheath. For example, the ends of the tethers securing the connection member to the prosthetic valve can be pulled in a distal direction, which in turn can squeeze, compress, and/or otherwise transition the connection member to a compressed or pinched configuration. With the connection member coupled to the supra-annular surface of the prosthetic valve the pre-compression and/or pre-tensioning of the connection member can pinch a proximal supra-annular portion of the prosthetic valve, which in turn, can start the process of transitioning the prosthetic valve from its deployment or expanded state to its delivery/retrieval or compressed state, as described above with reference to the connection member 478.

In some implementations, the method 10 optionally includes guiding, via the guide member, at least one subannular edge of the prosthetic valve as the prosthetic valve is pulled into the distal end of the retrieval sheath, at 17. For example, as described above with reference to the retrieval element 491, the guide member can be and/or can form a scoop, tongue, flange, and/or the like that can contact and/or guide a subannular portion of the valve into the retrieval sheath. More particularly, the subannular portion of the valve can include one or more edges and/or features that may, in some instances, be prone to snagging the distal end of the retrieval sheath. Accordingly, the guide member can be configured to guide such edges and/or features to limit and/or substantially prevent snagging the distal end of the retrieval. In this manner, the method 10 can be used to retrieve a prosthetic valve from the chamber of the heart. In some instances, with the valve retrieved and/or retracted into the retrieval sheath, the delivery/retrieval system can be retracted and removed from the body of the patient.

While various embodiments have been described above, it should be understood that they have been presented by way of example only, and not limitation. Likewise, it should be understood that the specific terminology used herein is for the purpose of describing particular embodiments and/or features or components thereof and is not intended to be limiting. Various modifications, changes, and/or variations in form and/or detail may be made without departing from the scope of the disclosure and/or without altering the function and/or advantages thereof unless expressly stated otherwise. Functionally equivalent embodiments, implementations, and/or methods, in addition to those enumerated herein, will be apparent to those skilled in the art from the foregoing descriptions and are intended to fall within the scope of the disclosure.

Where schematics, embodiments, and/or implementations described above indicate certain components arranged in certain orientations or positions, the arrangement of components may be modified. Although various embodiments have been described as having particular features and/or combinations of components, other embodiments are possible having a combination of any features and/or components from any of embodiments described herein, except mutually exclusive combinations. The embodiments described herein can include various combinations and/or sub-combinations of the functions, components, and/or features of the different embodiments described.

Where methods described above indicate certain events occurring in certain order, the ordering of certain events may be modified. Additionally, certain of the events may be performed concurrently in a parallel process, when possible, as well as performed sequentially as described above. While methods have been described as having particular steps and/or combinations of steps, other methods are possible having a combination of any steps from any of methods described herein, except mutually exclusive combinations and/or unless the context clearly states otherwise.

What is claimed:

1. A delivery/retrieval system for a side-deliverable prosthetic valve, the system comprising:
a control device configured to removably attach to a supra-annular surface of the prosthetic valve, the control device operable to (i) exert a distally directed force to advance the prosthetic valve in a compressed configuration through a lumen of a delivery sheath and into a chamber of a heart and (ii) exert a proximally directed force to pull the prosthetic valve disposed in the chamber of the heart in an expanded configuration into a distal end of a retrieval sheath; and
a retrieval element extendable through the lumen of the retrieval sheath, a proximal end portion of the retrieval element coupleable to a proximal end portion of the control device, a distal end portion of the retrieval element forming an engagement member and a guide member, the engagement member configured to be engaged with and exert a proximally directed force on a proximal subannular anchoring element of the prosthetic valve to pull the prosthetic valve from the chamber of the heart into the distal end of the retrieval sheath, the guide member configured to guide at least one edge of the prosthetic valve as the prosthetic valve is pulled into the distal end of the retrieval sheath.

2. The system of claim 1, wherein the guide member is a self-expanding element formed from a shape-memory alloy.

3. The system of claim 2, wherein the guide member is in a first configuration when disposed in the retrieval sheath and is configured to transition to a second configuration when advanced beyond the distal end of the retrieval sheath into the chamber of the heart

4. The system of claim 3, further comprising:
an actuator extending through the retrieval element and coupled to at least the guide member, the actuator configured to actuate the guide member in response to a proximally directed force to transition the guide member from the second configuration to a third configuration in which at least a distal end portion of the guide member is pulled in a proximal direction.

5. The system of claim 1, wherein the delivery sheath has a first diameter and the retrieval sheath has a second diameter larger than the first diameter.

6. The system of claim 1, wherein the retrieval element includes a retrieval catheter, the engagement member and the guide member mounted at a distal end of the retrieval catheter, the retrieval catheter defining a lumen configured to receive at least a portion of the control device.

7. The system of claim 1, further comprising:
a retractor removably coupleable to a retrieval handle disposed at a proximal end of the retrieval sheath and the proximal end portion of each of the control device and the retrieval element, the retractor configured, in response to being actuated, to move each of the control device and the retrieval element in a proximal direction relative to the retrieval handle,
wherein moving the control device and the retrieval element in the proximal direction exerts the proximally directed force on the prosthetic valve to pull the prosthetic valve into the distal end of the retrieval sheath.

8. A delivery/retrieval system for a side-deliverable prosthetic valve, the system comprising:
a retrieval sheath defining a lumen;
a control device extendable through the lumen of the retrieval sheath and removably coupleable to a supra-annular surface of the prosthetic valve;
a retrieval element extendable through the lumen of the retrieval sheath and outside of the control device, a distal end portion of the retrieval element including an engagement member configured to engage a proximal subannular anchoring element of the prosthetic valve when the prosthetic valve is at least partially disposed in a chamber of a heart,
wherein the control device being removably coupled to the supra-annular surface of the prosthetic valve and the engagement member being engaged with the proximal subannular anchoring element allowing the control device and the retrieval element to pull the prosthetic valve into a distal end of the retrieval sheath in response to a proximally directed force.

9. The system of claim 8, wherein the retrieval element includes a retrieval catheter that defines a lumen configured to receive a portion of the control device.

10. The system of claim 8, wherein the retrieval element includes a retrieval catheter, the engagement member is coupled to and extends distally from a distal end of the retrieval catheter.

11. The system of claim 10, wherein the retrieval element further includes a guide member that is coupled to and extends distally from the distal end of the retrieval catheter, the guide member configured to guide a subannular region of the prosthetic valve into the distal end of the retrieval sheath.

12. The system of claim 11, wherein the guide member is a self-expanding element formed from a shape-memory alloy, the guide member having a compressed configuration for delivery via the retrieval sheath, the guide member having an expanded configuration when released from the retrieval sheath, the guide member in the expanded configuration having a curved shape with a radius of curvature.

13. The system of claim 12, further comprising:
an actuator extending through the retrieval element and coupled to at least the guide member, the actuator configured to actuate the guide member in response to a proximally directed force to decrease the radius of curvature of the guide member.

14. The system of claim 11, wherein the engagement member is partially embedded in the guide member, a portion of the engagement member forming a hook that extends outwardly from the guide member allowing the portion of the engagement member to engage the proximal subannular anchoring element.

15. The system of claim 9, further comprising:
a retrieval handle coupled to a proximal end of the retrieval sheath; and
a retractor removably coupleable to the retrieval handle and a proximal end portion of each of the control device and the retrieval element, the retractor configured, in response to being actuated, to collectively move the control device and the retrieval element in a proximal direction relative to the retrieval handle,
wherein moving the control device and the retrieval element in the proximal direction exerts the proximally directed force on the prosthetic valve to pull the prosthetic valve into the distal end of the retrieval sheath.

16. The system of claim 15, wherein the retractor is configured such that the proximally directed force exerted on the prosthetic valve as a result of collectively moving the control device and the retrieval element in the proximal direction is sufficient to transition the prosthetic valve from a deployment configuration to a delivery configuration as the prosthetic valve is pulled into the distal end of the retrieval sheath.

17. An apparatus for retrieving a side-deliverable prosthetic valve from a chamber of a heart of a patient, the apparatus comprising:
a retrieval catheter;
a guide member coupled to and extending distally from a distal end of the retrieval catheter, the guide member formed from a braided tube of a shape-memory alloy, the guide member having a compressed state for delivery through a retrieval sheath into the chamber of the heart and an expanded state when in the chamber of the heart and distal to the retrieval sheath;
an engagement member coupled to and extending distally from the distal end of the retrieval catheter, the engagement member partially embedded in the guide member such that a portion of the engagement member extends outwardly from the guide member allowing the portion of the engagement member to engage a subannular region of the prosthetic valve.

18. The apparatus of claim 17, wherein the guide member in the expanded state having a scoop shape.

19. The apparatus of claim 18, further comprising:
an actuator extending through the retrieval catheter and coupled to at least the guide member, the actuator configured to actuate the guide member in response to a proximally directed force to decrease a radius of curvature associated with the scoop shape.

20. The apparatus of claim 17, wherein the portion of the engagement member is a hook configured to hook onto a proximal subannular anchoring element of the prosthetic valve.

21. The apparatus of claim 17, wherein the retrieval catheter defines a lumen configured to receive a control device such that a connection member at a distal end portion of the control device is distal to the retrieval catheter and removably coupleable to a supra-annular surface of the prosthetic valve.

22. The apparatus of claim 21, wherein the portion of the engagement member configured to engage a proximal subannular anchoring element of the prosthetic valve such that a proximally directed force exerted collectively on a proximal end portion of the retrieval catheter and a proximal end portion of the control device is operable to pull the prosthetic valve into a distal end of the retrieval sheath.

23. A method of using a delivery/retrieval system for retrieving a side-deliverable prosthetic valve at least partially disposed in a chamber of a heart of a patient, the method comprising:
decoupling a delivery portion of the delivery/retrieval system from a proximal end portion of a control device disposed outside the patient, a distal end portion of the control device disposed in the chamber of the heart and removably coupled to a supra-annular surface of the prosthetic valve;
advancing a retrieval sheath over the control device to place a distal end portion of a retrieval element distal to the retrieval sheath in the chamber of the heart, the distal end portion of the retrieval element including an engagement member;
coupling a first portion of a retractor to a proximal end portion of the retrieval sheath and a second portion of the retractor to a proximal end portion of each of the control device and the retrieval element;
engaging a proximal subannular anchoring element of the prosthetic valve with the engagement member of the retrieval element; and
actuating the retractor to move each of the control device and the retrieval element in a proximal direction relative to the retrieval sheath and to pull the prosthetic valve into a distal end of the retrieval sheath.

24. The method of claim 23, wherein the distal end portion of the control device includes a connection member removably coupleable to the supra-annular surface of the prosthetic valve via a set of tethers.

25. The method of claim 24, further comprising:
coupling an exchange catheter to the proximal end portion of the control device; and
securing end portions of each tether from the set of tethers via the exchange catheter.

26. The method of claim 24, further comprising:
exerting a proximally directed force on end portions of each tether from the set of tethers to transition the connection member to a compressed state prior to actuating the retractor.

27. The method of claim 23, wherein the distal end portion of the retrieval element includes a guide member, the method further comprising:
guiding, via the guide member, at least one subannular edge of the prosthetic valve as the prosthetic valve is pulled into the distal end of the retrieval sheath.

28. The method of claim 27, further comprising:
actuating at least a distal end portion of the guide member after the advancing the retrieval sheath, the actuating pulling at least the distal end portion of the guide member in the proximal direction; and
advancing the retrieval element relative to the retrieval sheath to place the engagement member in a distal position relative to the proximal subannular anchoring element, and
wherein the engaging the proximal subannular anchoring element with the engagement member includes moving the retrieval element in a proximal direction such that the engagement member engages the proximal subannular anchoring element.

29. The method of claim 23, wherein the advancing the retrieval sheath over the control device includes advancing a dilator over the control device to facilitate movement of the distal end of the retrieval sheath through a vasculature of a patient and into the chamber of the heart, the method further comprising:
withdrawing the dilator from the patient after the distal end of the retrieval sheath is in the chamber of the heart; and
advancing the retrieval element over the control device and through the retrieval sheath to place the distal end portion of the retrieval element distal to the retrieval sheath in the chamber of the heart.

30. The method of claim 23, wherein the proximal end portion of the retrieval sheath is coupled to a retrieval handle, the coupling the first portion of the retractor includes coupling the first portion of the retractor to the retrieval handle, and wherein the proximal end portion of the retrieval element is coupled to the proximal end portion of the control device via a coupler, the coupling the second portion of the retractor includes coupling the second portion of the retractor to the coupler.
